(19) United States
(12) Patent Application Publication
(10) Pub. No.: US 2021/0162047 A1
(43) Pub. Date: Jun. 3, 2021

(54) A Drug For Treating Disorders Of An Organ Or Tissue Function And Diseases Accompanied By Such Disorders, And The Method For Obtaining It.

(71) Applicant: Oleg IIiich EPSHTEIN, Moscow (RU)

(72) Inventors: Oleg Iliich EPSHTEIN, Moscow (RU); Sergey Aleksandrovich TARASOV, Vniissok (RU)

(21) Appl. No.: 17/057,624

(22) PCT Filed: Apr. 29, 2019
(86) PCT No.: PCT/RU2019/050057

§ 371 Date: Nov. 20, 2020

(30) Foreign Application Priority Data
May 22, 2018 (RU) 2018118695

(51) Int. Cl. A61K 41/00; A61K 39/395; A61B 5/318; A61B 5/369; A61B 5/398; A61B 5/392; A61B 5/0535; A61B 5/30

(52) U.S. Cl. CPC A61K 41/0004; A61K 39/3955; A61B 5/318; A61B 5/30; A61B 5/398; A61B 5/392; A61B 5/0535; A61B 5/369

(57) ABSTRACT

Summary The claimed medicament relates to medicine, and it can be used to treat disordered functions of organs or tissues, as well as a wide range of diseases accompanied by disordering of the functions of organs or tissues. The claimed medicament contains the original drug substance or the released-active form of the drug substance treated with bioelectric potentials which have been taken reading from the respective organ or tissue with normal functioning. The pharmaceutical drug substance or the released-active drug substance treated with bioelectric potentials can further be undergo successive multiple dilutions in a solvent selected from the group comprising water and a mixture of water and. The method of obtaining the drug involves exposing the drug substance or the released-active form of the drug substance to bioelectric potentials taken reading from the organ or tissue with normal functioning, with a further, optional successive multiple dilutions in a solvent.

The invention relates to medicine and can be used to treat a wide range of diseases associated with disordered (impaired) organs or tissues functions or diseases accompanied by disorders of organs or tissues functions.

Various methods of recording biopotentials from organs and tissues are known from the prior art such as:

Electroencephalography (EEG) method of recording changes in the brain's electric potentials using electrodes attached to the overlying scalp and connected to a series of amplifiers, filters and data acquisition systems (see Zenkov L. R., Clinical electroencephalography (including epileptology elements) [in Russian], 3 ed. Moscow: MEDpress-inform, 2004. 368 p.).

Electroretinography a method used to evaluate retinal function by measuring biopotentials generated in the retina following light exposure [Shamnishova A. M. Electroretinography in ophthalmology [in Russian], Moscow: Medika, 2009. 304 p.].

Electrogastroenterography (EGEG) is a method for evaluating gastric motility and evacuation by simultaneous recording of biopotentials from different sites of the gastrointestinal tract [Modern investigational approaches in gastroenterology [in Russian] (ed. V. K. Vasilenko). Moscow: Meditsina, 1971. 400 p.].

Electromyography is a method used to measure biopotentials in human and animal skeletal muscles following excitation of the muscle fiber [Nikolaev S. G. Electromyography Clinical practical guide [in Russian]. Ivanolvo, 2013].

Impedance plethysmography is a method of studying blood volume pulses in the vessels of various organs and tissues by graphically recording changes in the impedance of the tissue. It is used to detect various vascular abnormalities of the brain, extremities, lungs, heart, liver, etc. [Ronkin M. A., Ivanov L. V. Impedance plethysmography in clinical practice [in Russian]. Moscow: MBN, 1997. 403 p.].

Electrocardiography is a method of recording and examining the electric fields generated by the heart as it functions [Zudbinov Y. I. The basics of ECG [in Russian]. Rostov-on-Don: Feniks, 2003. 160 p.].

Electrodermal activity (skin conductance response) (EDA) is a bioelectric response recorded from the surface of the skin, and is widely used in psychophysiology as a measure of the activity of the autonomic nervous system [Aldersons A. A. The mechanisms of electrodermal reactions. Riga, 1985].

Although all the above methods are well-known in medicine and used to diagnose various diseases of the organs and tissues which are capable of generating biopotentials, none of them are utilized for treating diseases associated with disordered/impaired organ or tissue function.

An agent with an effect on the body has been known in the prior art, which consists of a neutral liquid carrier (e.g., distilled water or alcohol) treated by exposure to induced energy oscillations with frequencies and amplitudes proportional to those of biopotentials recorded from the body, and then potentized by successive multiple (serial) dilutions and succussion (shaking) steps according to the homeopathic method (RU 2140253 C1, A 61 J 3/00, 1999). The energy treatment is accomplished by exciting mechanical acoustic vibrations in the initial neutral carrier material, using for instance an electrostrictive element or a membrane, or by generating electromagnetic waves, for example using an electromagnetic coil. However, the effectiveness of said agent is limited due to a lack of specificity to target organs and tissues.

In the prior art, numerous drug products used to treat impaired organ or tissue function have been known, which, apart from their therapeutic action, produce adverse effects and have contraindications for use, for example as follows.

Drug product “Ganaton” intended to treat impaired gastrointestinal function. The use of the product can be associated with side effects such as leukopenia, thrombocytopenia, urticaria, anaphylactoid reaction, gynecomastia, increased prolactin level, increased salivation, nausea, diarrhea, constipation, jaundice, headache, dizziness, tremor, etc. The product is contraindicated in: hypersensitivity to itopride (or any other excipient in the product's formulation), GI hemorrhage, obstruction or perforation, pregnancy, lactation, and children under 16 years of age. It should be used with special caution by individuals with underlying conditions that can be worsened by cholinergic adverse reactions (associated with the effect of acetylcholin enhanced by itopride) [https://www.rlsnet.ru/tn_index_id_36285.htm].

The use of drug product “Adaptol”, indicated for impaired brain function, may be associated with the following side effects: dizziness, decreased blood pressure, dyspeptic disturbances, allergic reactions (rash, itching), bronchial spasm, hypothermia, fatigue, etc. The product is contraindicated in hypersensitivity to any of its components, pregnancy, lactation, and children under the age of 18 years [https://www.rlsnet.ru/tn_index_id_14402.htm].

“Mesaton”, a drug product for treating ocular dysfunction, has the following side effects: increased blood pressure, palpitation, ventricular fibrillation, arrhythmia, bradycardia, precordial pain, dizziness, fear, insomnia, restlessness, fatigue, headache, tremor, paresthesias, convulsion, cerebral hemorrhage, facial pallor, skin ischemia at the site of injection, very rare cases of necrosis and incrustation when the medicament penetrates the tissue or injected subcutaneously, and allergic reactions. The product is contraindicated in hypersensitivity to any of its components, hypertrophic obstructive cardiomyopathy, pheochromocytoma, and ventricular fibrillation. It should be used with special caution in metabolic acidosis, hypercapnia, hypoxia, atrial fibrillation, closed-angle glaucoma, arterial hypertension, pulmonary hypertension, hypovolemia, severe aortic stenosis, acute myocardial infarction, tachyarrhythmia, ventricular arrhythmia, arterial thromboembolism, atherosclerosis, thromboangiitis obliterans (Buerger disease), Raynaud syndrome, frequent vascular spasm (incl. on cold injury), diabetic endarteritis, thyroid dysfunction, diabetes mellitus, porphyria, glucose-6-phosphate dehydrogenase deficiency, combined use of monoamine oxidase inhibitors, general (halothane) anesthesia, impaired renal function, elderly patients, and patients under the age of 18 years. [https://www.rlsnet.ru/tn_index_id_4759.htm].

Thioridazine, used in the treatment of muscular dysfunction, is associated with the following side effects: drowsiness, syncope, feeling confused, impaired psychomotor function, tardive dyskinesia, agitation, excitement, insomnia, hallucinations, increased psychotic behaviours, extrapyramidal symptoms of dystonia or parkinsonism, emotional disturbances, impaired thermal regulation, lowered convulsion threshold, photophobia, impaired vision, hyposaivation, tongue papillary hypertrophy, increased/decreased appetite, dyspepsia, nausea, vomiting, constipation/diarrhea, paralytic ileus, cholestatic hepatitis, galactorrhea, breast engorgement, amenorrhea, dysmenorrhea, hyperprolactinaemia, gynecomastia, false positive pregnancy test results, weight gain, decreased blood pressure, tachycardia, non-specific ECG changes, suppression of bone marrow hematopoiesis (agranulocytosis, leukopenia, granulocytopenia, eosinophilia, thrombocytopenia, aplastic anaemia, pancytopenia), bronchial spasm, nasal congestion, ischuria paradoxa, dysuria, lowered libido, sexual dysfunction (incl. retrograde ejaculation), priapism, rash (incl. erythematous eruption), angioedema, exfoliative dermatitis, peripheral edema, parotid gland swelling, melanosis cutis (with prolonged use of high dosages). The contraindications for its use include hypersensitivity, severe heart disorders such as decompensated chronic heart failure or severe hyper- or hypotension, markedly suppressed CNS function, comas of any etiology, traumatic brain injury, progressive systemic diseases of the brain and spinal cord, concomitant use of medications that prolong the QT-interval, long QT syndrome, arrhythmias (incl. past ones), concomitant use of inhibitors and substrates of CYP2D6, porphyria, pregnancy, breast-feeding, and infant age <2 years [https://www.rlsnet.ru/mnn_index_id_1277.htm].

“Advantan”, a drug product used to treat impaired skin function, has side effects such as itching, burning, redness, the development of vesicular rash, skin atrophy, telangiectasias, stretch marks, acne, infection of hair follicles, excessive unwanted hair growth, perioral dermatitis, skin discolouration, allergic skin reactions to any of the ingredients of the formulations. The contraindications include infection at the site of application due to tuberculosis or syphilis; or a virus such as chicken-pox or shingles, rosacea, perioral dermatitis at the site of application, infant age <4 months, a skin reaction following a vaccination, and hypersensitivity to any of the product's ingredients [https://www.rlsnet.ru/tn_index_id_60.htm].

“Belloid” is a drug product used to treat impaired cardiac function. Its possible side effects include increased intraocular pressure, difficulty focusing eyesight, increased heart rate (tachycardia), facial skin reddening, drowsiness, and a coma. A number of contraindications for its use should be considered as well, including hypersensitivity to any of the ingredients in the formulation, benign prostatic hyperplasia, arthrosis of any stage, any signs of injury or disease of the peripheral nerves, and end-stage glaucoma [https://www.rlsnet.ru/tn_index_id_512. htm]. Drug product “Aminazine”, for treating peripheral cardiac function impairment, has contraindications such as diseases of liver or kidneys, decompensated heart failure, marked hypotension, impaired gastric function, and gastric and duodenal ulcers. Its possible side effects include nausea, vomiting, arterial hypotension (up to loss of consciousness), parkinsonism, photosensitivity, and allergic reactions [https://www.rlsnet.ru/tn_index_id_4155.htm].

In the prior art, drugs have been known which are prepared as activated/potentiated forms of antibodies, and which have been used to treat medical conditions. In particular, there is a pharmaceutical composition containing activated/potentiated histamine antibodies, tumor necrosis factor alpha (TNF-α) antibodies, and S100 brain protein antibodies [RU patent No. 2500427], or there is a drug that contains activated/potentiated S100 brain protein antibodies [RU patent No. 2503462]. These have a limited number of indications.

Therefore, the technical effect of the invention is to develop an effective drug for treating a wide range of impairments of organ or tissue function and diseases accompanied by impaired organ or tissue function, with minimum side effects and contraindications.

The claimed effect is achieved by a medicament for treating disordered organ or tissue function (or diseases accompanied by impaired organ or tissue function) which comprises the original drug substance or the released-active form of the drug substance treated with bioelectric potentials taken reading (acquired) from the above organ or tissue with normal functioning.

A drug substance is a drug represented by an active substance of biological, biotechnological, mineral or chemical origin which processes pharmacological activity and which is intended for use in the production and manufacture of medicinal products, and measuring their efficacy [Federal Act of the Russian Federation No. 61 (61-FZ) “On medicinal product circulation” dated 12 Apr. 2010]. Alternatively, the drug substance may be monoclonal, polyclonal, or natural antibodies.

Alternatively, examples of the drug substance may include, but are not limited to, monoclonal, polyclonal, or natural antibodies to interferon gamma, S100 brain protein, angiotensin II receptor, endothelial NO-synthase, or human cannabinoid receptor—used either individually or as a combination.

Alternatively examples of the drug substance may include, but are not limited to, the released-active form of monoclonal, polyclonal, or natural antibodies to interferon gamma, S100 brain protein, angiotensin II receptor, endothelial NO-synthase, or human cannabinoid receptor—used either individually or as a combination.

Alternatively, examples of the drug substance may include, but are not limited to, a drug or a combination of drugs.

Alternatively, examples of the drug substance may include, but are not limited to, a combination of released-active forms of a drug or the released-active form of a combination of drugs.

“A normal organ or tissue” means an organ or tissue which has no impairment to its functions on diagnostic testing with adequate techniques.

The term “the released-active form of the drug substance” is synonymous to “activated—potentiated form of the drug substance”, as well as to “ultra-low dose of the drug substance” and is used to denote a product obtained by successive multiple (serial) dilutions of a drug substance stock (matrix) solution, combined with external treatment of each serial dilution, in particular by mechanical shaking. The process utilized to prepare the released-active form of the drug substance is the one used to obtain the activated-potentiated form of the drug substance, respectively [WO2012017324, U.S. Pat. Nos. 7,229,648 and 4,311,897], and therefore it can also be referred to as an activation—potentiation process. The external treatment during the concentration reduction process can also be provided by, for example exposure to ultrasonic, electromagnetic or other physical factors. [Schwabe V. Homeopathic medicines [in Russian]. Moscow, 1967; U.S. Pat. Nos. 7,229,648 and 4,311,889].

Said procedure uniformly decreases the molecular concentration of the initial molecular form of the drug substance, and it is repeated until the required dilution is obtained. For an individual drug substance, the target dilution can be defined by evaluating the biological activity of intermediate dilutions using an adequate model.

Dilutions used in released-active forms are labeled by Latin letters (e.g. a decimal dilution is labeled D, and a centesimal dilution is designated as C, etc.) followed by a number which indicates the number of repeat dilution steps employed. [Schwabe V. Homeopathic medicines [in Russian]. Moscow, 1967; U.S. Pat. Nos. 7,229,648 and 4,311,889].

In particular, in order to prepare a 12-fold centesimal dilution (designated as C12) of interferon gamma (IFNγ) antibodies (Ab), one part of a 3.0 mg/mL IFNγ Ab stock is diluted in 99 parts of a neutral aqueous or aqueous alcoholic solvent, with subsequent steps of vigorous shaking (at least 10 repeat steps), producing thereby one C1 dilution. The next (C2) dilution will be prepared likewise from the C1 dilution, with the procedure repeated 11 times to obtain C12. So, a C12 dilution is a solution resulting from one part of 3.0 mg/mL IFNγ Ab stock diluted serially 12 times in 99 parts of a neutral solvent (each time in a separate container), which is ultimately equivalent to C12. Similar procedures, given the appropriate number of dilution steps, are performed to obtain C30, C50, C200 and other dilutions. The activity of intermediate dilutions can be tested using a desired biological model.

The drug substance or the released-active form of the drug substance, treated with bioelectric potentials, may be a combination of several drug substances and/or released-active forms of several drug substances.

The claimed medicament (drug) can be available as a solid dosage (powder, granules, or tablets) comprising a technologically appropriate (efficient) amount of a neutral carrier material, such as lactose saturated with a liquid form of the claimed medicament, and pharmaceutically acceptable excipients, preferably lactose, microcrystalline cellulose, and magnesium stearate.

The solid tablet form is produced in a fluidized bed system (such as Hüttlin Pilotlab from Mifflin GmbH) by spraying a previously prepared, preferably at the ratio of 1 kg of solution to 5 or 10 kg of lactose (1:5-1:10), aqueous or aqueous alcoholic solution of the claimed medicament onto a powdered (particle size, 50±500 μm) neutral matter, e.g. lactose (milk sugar), to saturation and drying it simultaneously with heated air being supplied onto the machine's bottom grid. A predefined amount of lactose (10±91% of total tablet blend) saturated with the liquid form of the claimed medicament as described above is loaded into a blender and processed with the addition of lactose pre-wetted with the liquid form of the claimed medicament (3±10% of total tablet blend) and unprocessed lactose (84% of tablet blend) (for the purpose of cost reduction and a more simplified and faster process, with the strength of therapeutic effect unchanged). Microcrystalline cellulose (5±10% of total tablet blend) and magnesium stearate (1% of tablet blend) are added afterwards. The resultant blend is mixed homogeneously, after which it is dry compressed (e.g., on a tablet press) into 150±500 mg tablets. The compression stage produces tablets comprising lactose saturated with the liquid form of the claimed drug.

Alternatively, the drug substance or the released-active form of the drug substance treated with bioelectric potentials is further undergo with successive multiple dilutions in a solvent selected from the group comprising water and a mixture of water and alcohol. The additional serial dilution stage can involve external mechanical treatment of each dilution, such as shaking.

Alternatively, the claimed medicament can be prepared by trituration, wherein said stock (matrix) solution, previously prepared as a liquid form of the energy-treated drug substance or released-active form of the drug substance, is “dry diluted”. “Dry dilution” is a process wherein a matrix trituration is prepared by wetting a dry neutral carrier material (preferably lactose (milk sugar) granules) with liquid energy-treated drug substance or released-active form of the drug substance and then serially ground (i.e. comminuted thoroughly) so that 1 weight part of each trituration, starting with the matrix one, is comminuted with 9 (for a decimal (D) trituration) or 99 (centesimal (C) trituration), or 999 weight parts (millesimal (M) trituration) of the neutral carrier, with each serial trituration prepared in a separate container until the target trituration (according to the relevant homeopathic potency scale) is obtained (see, for example Schwabe V. Homeopathic medicines [in Russian]. Moscow, 1967. pp. 20-29).

The released-active (activated-potentiated) form of drug substance is characterized by the following. First, it is contemplated that the released-active (activated-potentiated) form of drug substance possesses biological activity detectable by up-to-date standard pharmacological techniques. Second, it is contemplated that the biological activity of the released-active (activated-potentiated) form of drug substance cannot directly be attributed to the presence of molecular form of the drug substance in the end product.

The biological activity of the released-active form of drug substance observed in any dilution, in particular those described in PCT/IB2011/002177 (p. 41, line 25), as the synergic effects of different dilutions of the same drug substance has been positively evidenced by studies carried out at scientific institutions, and described, in particular in an article titled “Dose-dependent effects and action specificity of ultra-low doses of antibodies to endogenous regulators (Bull. Exp. Biol. Med., (5/2): 527-529). For instance, C3 and C12 dilutions of antibodies to IFNγ, as well as the mixture of C12+C30+C50 dilutions thereof have been shown to increase the production of endogenous IFNγ. The article also presents evidence that the biological activity of the released-active form of drug substances has specificity.

Bioelectric potentials (biocurrents) are electric phenomena that occur in resting or physiologically active living cells. Bioelectric potentials of different cell types can add up into an electrical response of an entire tissue or organ. If the number cells of a tissue of interest that get into excited state at one time and start generating action potentials or other electrical response is big enough, an electrical response can be recorded from the tissue as a whole (e.g. a nerve, muscle, gland, brain region, etc.). (http://www. medical-enc.ru/2/biotoki.shtml).

Bioelectric responses can be taken reading (acquired) by any known method such as, for example an electrophysiological method for recording bioelectric potentials generated by the functioning of the relevant organ or tissue.

In the claimed invention, evoked potentials can also be used. An evoked potential is an electrical response of an organ (predominantly the brain) to an external stimulus or cognitive challenge [Shagass C. Evoked potentials in normality and pathology [translation into Russian], 1975].

Alternatively, the bioelectric potentials can be acquired from the organ or tissue of one or more healthy donors.

A “donor” (or “healthy donors”) means a living subject in which the above organ or tissue functions normally.

Furthermore, bioelectric potentials taken reading (acquired) from more than one donor are averaged (summed) before being used for treating the drug substance. The currents are summed as described by Kirchhoff s first law, i.e. in parallel circuits of biopotential sources. [Shilo V. L. Linear integrated circuits in radio-electronic hardware (ed. Galperin E. I.) [in Russian]].

The drug substance can be treated by exposure to an electric current resulting from a potential difference applied to electrodes immersed in the drug substance or the released-active form of drug substance and proportional to the difference in recorded bioelectric potentials occurring in the donor's organ or tissue as it functions.

Furthermore, the potential difference applied to at least two electrodes immersed in the solution containing the drug substance or the released-active form of drug substance is proportional in amplitude to the amplified total output produced by summing the bioelectric potential differences from all electrodes employed by an electrophysiological method for recording biopotentials occurring in a donor's organ or tissue as it functions.

The organ or tissue can be either that of a living being or a separate (isolated) one.

Isolated organs are body parts or organs (muscle, kidney, heart, extremity, etc.) separated from the body and placed in an artificial growth medium, after which they temporarily preserve their major functional properties. They are used in experimentation in physiology, biochemistry and pathology. [The Russian Big Encyclopaedic Dictionary, 2000].

The claimed invention can be used to treat impaired function of a body organ and/or tissue or diseases accompanied by or related to impaired organ and/or tissue function.

The organ to be treated with the claimed medicament may be the heart. Electrocardiography will then be used as the method for recording biopotentials, with impaired cardiac function incorporating:

Cardiac disorders accompanied by abnormal heart rhythms such as: sinus tachycardia; sinus bradycardia; sinus arrhythmia—tachyarrhythmia or bradyrhythmia; premature atrial contractions; paroxysmal atrial tachycardia; junctional extrasystole; paroxysmal tachycardia, premature ventricular contractions; paroxysmal ventricular tachycardia; atrial and ventricular fibrillation and flutter;

Cardiac disorders accompanied by conduction abnormalities:

sinoatrial block, intra-atrial block; atriventricular block, first, second, and third degree; bundle branch block;

Cardiac conditions associated with: ischemic heart disease, including angina, acute or prior myocardial infarction; high blood pressure; thrombosis, heart malformation; cardiomyopathies (structural abnormalities in myocardial anatomy), including those resulting from the above-listed disorders;

Heart failure;

Hypertension (essential or symptomatic);

Inflammation of heart membranes: pericarditis, myocarditis, endocarditis;

Pulmonary heart disease and diseases of pulmonary circulation;

Chronic rheumatic heart diseases; other diseases accompanied by impaired heart function.

Another example of the organ to be treated with the claimed medicament may be the gastrointestinal tract. In this case, electrogastoenterography will be used as the biopotential recording method while impaired organ function should be understood as functional gastrointestinal disorders, such as:

1. Functional esophageal disorders: functional chest pain; functional heartburn; reflux hypersensitivity; globus; and fucntional dysphalgia.

2. Gastroduodenal disorders: functional dyspepsia; postprandial distress syndrome; and epigastric pain syndrome.

3. Belching disorders: excessive supragastric belching, excessive gastric belching; excessive gastric belching; nausea and vomiting disorders; chronic nausea vomiting syndrome; cyclic vomiting syndrome; cannabinoid hyperemesis syndrome; and rumination syndrome.

4. Bowel disorders: irritable bowel syndrome (IBS) including:

constipation-predominant IBS (IBS-C);

diarrhea-predominant IBS (IBS-D);

IBS with mixed stool pattern (IBS-M);

untyped IBS (IBS-U).

functional constipation; functional diarrhea; functional abdominal bloating/distension; unspecified functional bowel disorder; and opioid-induced constipation.

5. Centrally mediated disorders of gastrointestinal pain such as centrally mediated abdominal pain syndrome; narcotic bowel syndrome/opioid-induced GI hyperalgesia.

6. Gallbladder and sphincter of Oddi disorders: biliary pain including:

a) functional gallbladder disorder;

b) functional biliary sphincter of Oddi disorder;

functional pancreatic sphincter of Oddi disorder.

7. Anorectal disorders: fecal incontinence; functional anorectal pain including:

a) levator ani syndrome;

b) unspecified functional anorectal pain;

c) proctalgia fugax;

functional defecation disorders including:

d) inadequate defecatory propulsion;

e) dyssynergic defecation.

8. Childhood functional GI disorders: neonate/toddler, such as infant regurgitation; rumination syndrome; cyclic vomiting syndrome (CVS); infant colic; functional diarrhea; infant dyschezia; and functional constipation.

9. Childhood functional GI disorders: child/adolescent, such as functional nausea and vomiting disorders including:

a) cyclic vomiting syndrome (CVS);

δ) functional nausea and functional vomiting;

c) rumination syndrome;

B) aerophagia.

10. Functional abdominal pain disorders such as functional dyspepsia (postprandial distress syndrome or epigastric pain syndrome; irritable bowel syndrome; abdominal migraine; and functional abdominal pain.

11. Functional defecation disorders including functional constipation; and nonretentive fecal incontinence.

12. Impaired gastrointestinal function in the following disorders: organic diseases of the stomach and duodenum accompanied by dyspeptic (gastric and duodenum erosive ulcers, cicatricial ulcers, gastroduodenal emptying abnormality, neoplasms, etc.); gastro-oesophageal reflux; abnormal abnormal gastroduodenal motility (biliary reflux); coeliac disease (gluten-sensitive enteropathy); lactase or other disaccharidase deficiency; exocrine pancreatic insufficiency; inflammatory bowel disease (Crohn's disease or ulcerative colitis); microscopic colitis (lymphocytic or collagenous); radiation colitis; NSAID-induced colitis; abdominal angina; colorectal cancer; acute infectious diarrhea; antibiotic-associated diarrhea and pseudomembranous colitis; giardiasis; small intestine bacterial overgrowth (SIBO) and other gastrointestinal disorders accompanied by impaired gastrointestinal tract function.

The tissue to be treated with the claimed medicament may be muscle. Electromyography will then be used as the biopotential recording method while impaired tissue function should be understood as impaired muscle function. Examples of such disorders can be writer cramp; tic disorders; stuttering; or abnormal muscle function in, for example, myositis; ossification and calcification of muscle; diastasis of muscle; ischaemic infarction of muscle (compartment syndrome); immobility syndrome (paraplegic); contracture of muscle; muscle hernia; and other diseases accompanied by impaired muscle function.

Another example of the tissue to be treated with the claimed medicament may be the retina. Electroretinography will be used as the biopotential recording method while impaired tissue function should be understood as impaired retinal function. Examples of such disorders may be spasm of accommodation; presbyopia; accommodation asthenopia; paresis of accommodation; or abnormal eye function in, for instance retinal detachments and breaks; retinal vascular occlusions; background retinopathy and retinal vascular changes; degeneration of macula and posterior pole; peripheral retinal degeneration; chorioretinal disorders; chorioretinal inflammation; diabetic retinopathy; atherosclerotic retinopathy; and other disorders accompanied by impaired eye function.

The organ to be treated with the claimed medicament may also be the brain. In this case, electromyography will be used as the biopotential recording method while impaired tissue function should be understood as impaired muscle function. Examples of such disorders may be as follows.

Impaired Nervous System Function:

movement disorders; disturbances observed in cerebrovascular accident (stroke); hemorrhagic stroke, thrombosis, disturbances observed as sequelae of cerebrovascular accident; disturbances observed in parkinsonism/Parkinson's disease; disturbances in amyotrophic lateral sclerosis; disturbances in myasthenia; and disturbances in cerebral palsy;

Sensory disturbances: disturbances in multiple sclerosis; disturbances in cerebrovascular accident; and disturbances in polyneuropathy;

Autonomic disturbances—dysautonomia;

Coordination disturbances: disturbances in multiple sclerosis; disturbances in polyneuropathy; and disturbances in cerebrovascular disease;

Impaired cognitive function: cognitive function impairment associated with atherosclerosis and/or manifested as vascular dementia; dementia; or Huntington's chorea;

Episodic and paroxysmal disorders: disorders due to epilepsy; epileptic seizures, common migraine;

Mental and behavioral disorders: disorders with psychotic symptoms; disorder with productive psychotic symptoms (schizophrenia, paranoid or hallucinatory-delusional syndrome in schizophrenia, paranoid or hallucinatory-delusional syndrome in organic brain disease; delirium, acute intoxication with delirium); disorders with negative (deficit-type) syndrome (emotional and volitional disturbances in schizophrenia; epileptic personality disorders; psycho-organic personality disorders); disorders with neurotic symptoms (associated with neurotic or pseudoneurotic disorders; generalized anxiety disorder; psychological stress disorder; obsessive-compulsive disorder); pseudoneurotic disorder in organic brain disease; pseudoneurotic disorder in chronic somatic disease; sleep disturbance in neurotic or pseudoneurotic disorders; depression in neurotic or pseudoneurotic disorders; behavioral disorders (attention deficit hyperactivity disorder; phobic anxiety disorder—fears and anxiety disorders specific to childhood; autism; pathological personality development, psychopathic behavior due to perinatal injury to the nervous system; disorders of adult behavior; behavioral disorders due to use of alcohol; various types of behavioral disorder in psychopathy; disorders associated with mental retardation—delayed psychological development in children (developmental delay); specific developmental disorders of speech and language; specific developmental disorders of scholastic skills; mixed specific disorders of psychological development; specific developmental disorder of motor function; impaired cognitive function in adults; disturbances in chronic cerebrovascular diseases/chronic vascular disorders of the brain; dementia in Alzheimer's disease; dementia in other diseases);

Sleep disturbance: in neurotic and pseudoneurotic disorders; in chronic cerebrovascular disease; in alcohol dependence; in a stress-related reaction, in healthy individuals; and in somatic diseases, violation of the sleep-wake cycle of healthy individuals.

Impaired cognitive function: developmental delay in children, speech development delay, Attention deficit and hyperactivity disorder (ADHD);

Behavioral disorders: various types in psychopathy; disorders due to use of alcohol.

Mood disorders: depression in endogenous mental disorders, including schizophrenia, organic brain disease; depression in neurotic and pseudoneurotic disorders; post-traumatic or reactive (situational) depression; and depression in chronic cerebrovascular disease in the elderly;

Neurotic and pseudoneurotic disorders;

Nature of remission in mental disorders;

Disorder of the autonomic nervous system: primary disorder; somatic symptom disorder.

Brain ischemia, including acute, persistent, transient or chronic ischemia, and sequelae of cerebrovascular accident, focal cerebral ischemia.

Cerebral palsy (ICP);

Multiple sclerosis;

Parkinsonism, including idiopathic parkinsonism, Parkinson's disease, parkinsonism syndrome;

Disorders of the peripheral nervous system: pain due to intervertebral disc disorder; other dorsopathies.

Down syndrome; Huntington's disease; Unstable remission in neurological disorders; Alcoholism; Schizophrenia; Neurotic and pseudoneurotic disorders; Depression; Impaired cognitive function; Attention deficit hyperactivity disorder in children; other diseases accompanied by impaired brain function.

Another example of the tissue to be treated by the claimed medicament may be the skin. In this case, EDA measurements will be used as the biopotential recording method while impaired tissue function should be understood as impaired skin function. Examples of such disorders may be neurodermatitis; hyperhidrosis; and other diseases and conditions accompanied by impaired skin function.

The organ to be treated with the claimed medicament may also be is a circulatory organ. Impedance plethysmography will then be used as the biopotential recording method while impaired tissue function should be understood as impaired muscle function. Examples of such disorders may be diseases involving abnormal vascular tone such as nerocirculatory or vegetative-vascular dystonia (somatoform autonomic dysfunction), polyneuropathies; hypertension; atherosclerosis; Raynaud syndrome; and other diseases accompanied by impaired vascular (circulatory) function.

Additionally, the claimed technical effect is achieved by the method of obtaining the claim medicament wherein the drug substance or the released-active form of drug substance is treated with bioelectric potentials picked up from a donor's normal organ or tissue.

Alternatively, the obtained medicament is further undergo successive multiple dilutions in a solvent selected from the group comprising water and a mixture of water and ethanol. The multiple dilutions process can also be accompanied by external treatment of each serial dilution, in particular by mechanical shaking.

Alternatively, the drug substance or the released-active form of the drug substance may be treated by exposure to an electric current resulting from a potential difference applied to electrodes immersed in the drug substance or the released-active form of drug substance and proportional to the potential difference recorded for bioelectric potentials in the donor's organ or tissue as it functions. The treatment time is established experimentally.

Alternatively, the bioelectric potentials may be acquired using an electrophysiological method for recording bioelectric potentials occurring in the donor's organ or tissue as it functions, wherein

the potentials acquired are amplified in each channel and the amplified outputs are converted into digital form and transmitted as digitized signal files to the memory of a programmable digital signal summing unit—signal summer, where the digital signals are added together into one composite digital signal bearing information on the sum of potential differences from all recorded electrodes, which is afterwards input to the digital-to-analog converter of an arbitrary waveform generator, where it is converted back into a summed analog signal to generate output signals in the form of voltages (potential difference) that approximate the waveform of the summed bioelectric potential difference, which are then fed onto at least two current electrodes placed in a container filled with the initial neutral carrier

The organ or tissue used to acquire a biopotential may be an organ in a living subject or a separate (isolated) organ.

An example of the organ to acquire a potential from may be the heart wherein the biopotential recording method may be electrocardiography.

Another example of the organ to acquire a potential from may be the gastrointestinal tract wherein the biopotential recording method may be electrogastroenterograpghy.

An example of the tissue to acquire a potential from may be muscle wherein the biopotential recording method may be electromyography.

Another example of the tissue to acquire a potential from may be the retina wherein the biopotential recording method may be electroretinography.

Another example of the organ to acquire a potential from may be the brain wherein the biopotential recording method may be electroencephalography

Another example of the tissue to acquire a potential from may be the skin wherein the biopotential recording method may be EDA measurement.

Another example of the organ to acquire a potential from may be a circulatory organ (in particular, blood vessels or the heart) wherein the biopotential recording method may be impedance plethysmography.

Alternatively, the drug substance may be an antibody.

Alternatively, examples of the drug substance include, but are not limited to, of monoclonal, polyclonal, or natural antibodies to targets such as interferon gamma, S100 brain protein, angiotensin II receptor, endothelial NO-synthase, human cannabinoid receptor—either individually or as a combination.

Alternatively, examples of the drug substance may include, but are not limited to, the released-active form of monoclonal, polyclonal, or natural antibodies to targets such as interferon gamma, S100 brain protein, angiotensin II receptor, endothelial NO-synthase, or human cannabinoid receptor—used either individually or as a combination. Alternatively, the drug substance may be represented by a drug or a combination of drugs with known therapeutic activity.

Alternatively, the drug substance may be a combination of released-active forms of a drug or the released-active form of a combination of drugs.

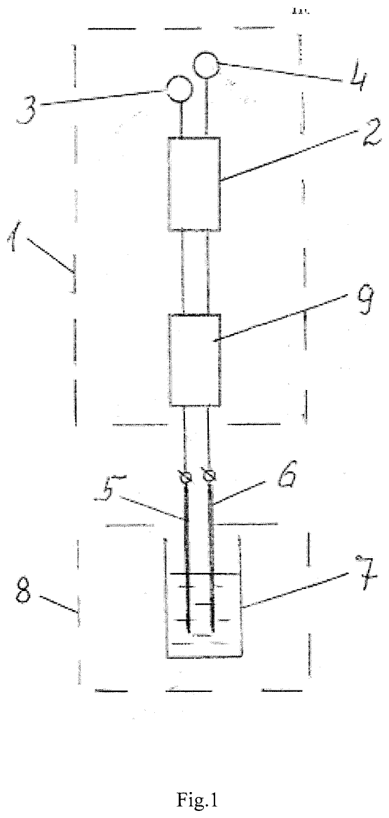

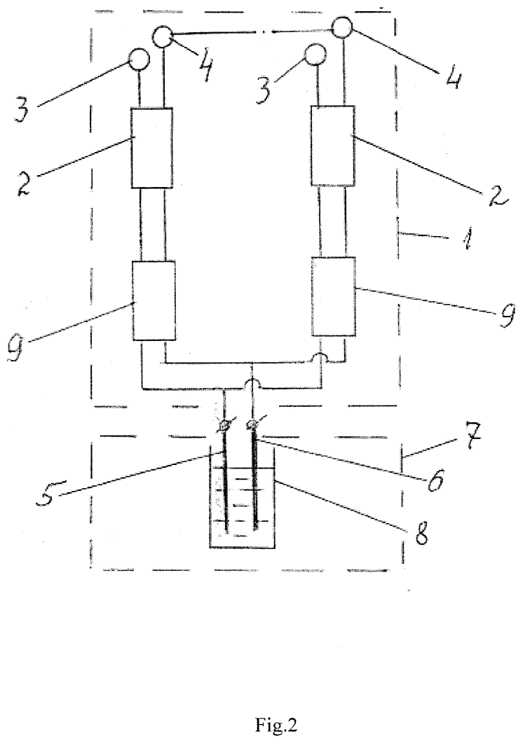

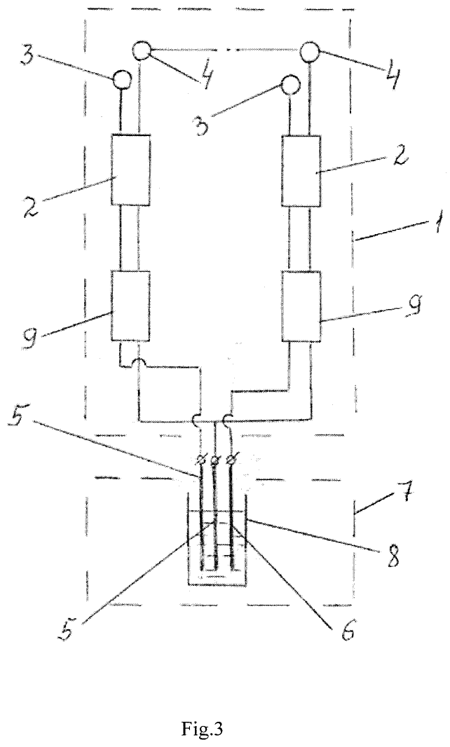

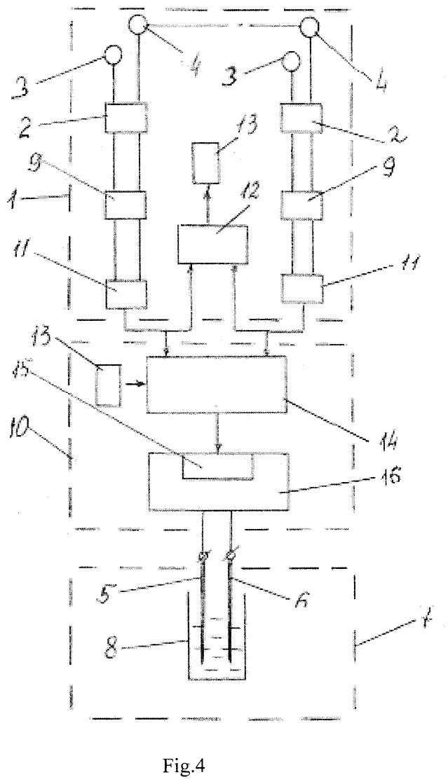

FIG. 1 shows a flow diagram that illustrates an embodiment of a device for producing the claimed drug, which comprises a pair of recording electrodes and a pair of current electrodes; FIG. 2 presents an alternative flow diagram of the device, which comprises several active and two current electrodes; FIG. 3 shows an alternative embodiment of the device for producing the claimed drug, which contains one reference recording electrode, several active recording electrodes, and several current electrodes; FIG. 4 shows an alternative embodiment of the device which comprises a digital data recording unit.

The claimed medicament can, for instance, be prepared as follows.

Initially, a scalp EEG is picked up from a donor who is placed during the measurement in a light- and sound-proof room and seated in a comfortable chair, with their eyes closed (according to the conventional EEG recording technique) Alternatively at least two electrodes (FIG. 1) of EEG recording unit 1 input the brain's electrical activity as a referential derivation of bioelectric potentials to channel amplifier 2, said referential derivation consisting of active scalp electrode 3 and reference electrode 4, generally positioned on the earlobe; or bioelectric potentials are input to channel amplifier 2 as a bipolar derivation consisting of active recording electrodes 3 placed at different scalp locations (not shown in the figure).

The amplified potentials (the peak amplitude of the output signal being between 0.5±1.5 volts) are then passed through low band-pass filter 9 with a bandwidth of 0.5 to 35 Hz and fed into two current electrodes 5 and 6 of energy treatment unit 7, which have been placed in container 8, thus providing primary energy treatment of the released-active form of interferon gamma, obtained by serially diluting the IFNγ Ab stock (matrix) solution by 100³⁰⁰ times (equivalent to a C300 dilution) in an aqueous solvent (concentration of 0.5 mg/mL), accompanied by external mechanical treatment—shaking of each serial dilution.

Distilled or purified (special purification grade) water or an aqueous alcoholic solution with a maximum conductivity of 0.1 mS/m at 25° C. is preferably used as the aqueous solvent.

Said energy treatment of the pharmaceutical released-active form of the drug substance is preferably carried out for at least 5 minutes.

The drug prepared by the above process (liquid energy-treated released-active form of the drug substance) can be used as a standalone treatment for, for example, neuropsychiatric diseases accompanied by impaired brain function, or as a stock (matrix) solution for further serial dilution in an aqueous or aqueous alcoholic solvent, possibly with additional external treatment (mechanical shaking of each serial dilution), in the preparation process used to obtain the claimed medicament for the treatment of, for instance, neuropsychiatric diseases accompanied by impaired brain function.

In another embodiment, the claimed medicament is obtained by exposing the drug substance or the released-active form of the drug substance to energy treatment such as an electric current created by a biolectric potential difference proportional in amplitude to the total bioelectric potential difference recorded with a bipolar and/or referential derivation from different sites on the normal brain (scalp locations) of a donor. In this case, an EEG is recorded by placing on the subject's head a silicone or textile cap with attached electrodes that pick up electrical activity from different scalp locations, and that incorporate, for example, based on the internationally recognized clinical 10-20 system, 19 recording electrodes 3 placed uniformly over the scalp and one reference electrode 4 located at a distance from the brain—generally on the earlobe (or a pair of short-circuited reference electrodes 4, each of which is attached to either earlobe).

Active recording scalp electrode 3 (overlying the brain) is connected to one input of each channel amplifier 2 (FIG. 2) while the other input of amplifiers 2 is connected to reference (zero) recording electrode 4, generally located on the earlobe (off the brain), wherein the amplified outputs from each channel amplifier 2 (the peak amplitude of the output signals being within 0.5±1.5 volts) are passed through low band-pass filter 9 with a bandwidth of 0.5 to 35 Hz and are fed in parallel into two current electrodes 5 and 6 of energy treatment unit 7 which have been placed in container 8 containing the initial neutral carrier, enabling the amplified potential differences obtained in the referential derivations to be summed or averaged right inside container 8.

Alternatively, (FIG. 3) active recording electrodes 3 (at different scalp locations overlying the brain) are connected to one input of several channel amplifiers 2, with the other input connected to reference (zero) recording electrode 4. The amplified potentials at the active outputs of each channel amplifier 2 are separately (or in lumps) passed through low band-pass filter 9 with a bandwidth of 0.5 to 35 Hz and fed into separate current electrodes 5 of energy treatment unit 7, wherein zero current electrode 6 receives short-circuited (joined together) zero outputs from channel amplifiers 2, enabling the potential differences obtained in the referential derivations to be summed or averaged right inside container 8.

Said energy treatment of the drug substance is preferable carried out for at least 5 minutes.

The outputs from channel amplifiers 2 can be connected to low band-pass filter 9 with a bandwidth of 0.5 to 35 Hz.

Preferably, distilled or purified (special purification grade) water or an aqueous alcoholic solution with a maximum conductivity of 0.1 mS/m at 25° C. is used as the solvent for the drug substance or the released-active form of the drug substance. As the drug substance, for instance, the released-active forms of monoclonal, polyclonal, or natural antibodies to interferon gamma, S100 brain protein, angiotensin II receptor, endothelial NO-synthase, or human cannabinoid receptor can be used—either individually or as a combination—or the antibodies proper or another drug.

The drug prepared by the above process (liquid energy-treated drug substance or released-active form of the drug substance) can be used as a standalone treatment for, for example, neuropsychiatric diseases accompanied by impaired brain function, or as a stock (matrix) solution for further successive multiple dilutions in an aqueous or aqueous alcoholic solvent, possibly with additional external treatment (mechanical shaking of each serial dilution), in the preparation process used to obtain the claimed medicament for the treatment of, for instance, neuropsychiatric diseases accompanied by impaired brain function,

In particular, the claimed medicament can also be prepared as follows.

Initially, EEGs are recorded as individual bioelectric potential differences picked up with independent referential derivations from different locations over the scalp of different, preferably healthy donors.

To do this, a silicone or textile cap is placed on the subject's head which contains attached electrodes picking up electrical activity from different scalp locations, which is input through referential derivations into multichannel amplifiers 2 of EEG recording unit 1 (FIG. 4). Preferably, based on the internationally recognized clinical 10-20 system, 19 recording electrodes 3 are used placed uniformly over the scalp and one reference electrode 4 located at a distance from the brain—generally on the earlobe (or a pair of short-circuited reference electrodes 4, each of which is attached to either earlobe). Active recording scalp electrode 3 (overlying the brain) is connected to one input of each channel amplifier 2, with the other input of each amplifier 2 connected to reference (zero) recording electrode 4, wherein the amplified output from each channel amplifier 2 (the peak amplitude of the output signals being between 0.5±1.5 volts) is passed through low band-pass filter 9 with a bandwidth of 0.5 to 35 Hz and input to respective digital-to-analog converter 11, where the analog signal is converted into digital form and transmitted as an individual digitized signal file representing an individual potential difference to the memory of programmable data recording unit 12 to be recorded onto electronic data storage device 13. Each individual EEG is recorded for 5÷10 minutes.

In the next step, the individual data recorded onto electronic storage devices 13 and/or digital outputs from analog-to-digital converters 11 are entered into the memory of programmable digital signal summing unit 14 (a minicomputer-based signal summer) of data processing unit 10 and added up using a preset program which enables generating an output as a combination of several individual digital files containing individual potential differences from EEGs (or EEG fragments) recorded independently in different subjects. From programmable digital signal summing unit 14—signal summer, the total composite signal representing the sum of potential differences recorded in individual referential derivations is input to digital-to-analog converter 15 of arbitrary waveform generator 16, where it is converted into a resultant analog signal to generate an output in the form of voltages (potential difference) that approximate the waveform of the total composite biopotential difference that incorporates all potential differences recorded with individual referential derivations from different donors, which is fed to at least two current electrodes 5 and 6 of energy treatment unit 7, placed in container 8 with the initial neutral carrier.

Preferably, distilled or purified (special purification grade) water or an aqueous alcoholic solution with a maximum conductivity of 0.1 mS/m at 25° C. is used as the initial, primary carrier. The drug substance, for instance, may be represented by released-active forms of monoclonal, polyclonal, or natural antibodies to interferon gamma, S100 brain protein, angiotensin II receptor, endothelial NO-synthase, or human cannabinoid receptor—either individually or as a combination—or the antibodies proper or another drug.

The drug prepared by the above process (liquid energy-treated drug substance or released-active form of the drug substance) can be used as a standalone treatment for, for example neuropsychiatric diseases accompanied by impaired brain function, or as a stock (matrix) solution for further successive multiple dilutions in an aqueous or aqueous alcoholic solvent, possibly with additional external treatment (mechanical shaking of each serial dilution), in the preparation process used to obtain the claimed medicament for the treatment of, for instance, neuropsychiatric diseases accompanied by impaired brain function,

Where the claimed medicament is produced by exposing the drug substance or the released-active form of the drug substance to bioelectric potentials acquired from other organs or tissues capable of generating biopotentials (e.g. eyes, skin, gastrointestinal organs, heart, circulatory organs, or muscle), a suitable recording method (using the appropriate type, number, and placement of electrodes, etc.) accepted in medical practice for the purposes in question is used. Once bioelectric potentials have been acquired, the drug substance or the released-active form of the drug substance is treated and the claimed medicament produced using the procedure described.

Thereafter the claimed medicament can be formulated into a solid dosage form (powder, granules or tablets) containing the technologically appropriate amount of neutral carrier.

Furthermore, the claimed technical effect is achieved by using the claimed medicament for indications known for the drug substance itself or the released-active form of the drug substance, or for treating impaired function of the organ or tissue from which the biopotentials have been acquired. The claimed medicament can exhibit its efficacy not only by enhancing the properties already known for the drug substance utilized or the released-active form of the drug substance but also by acquiring new properties, providing more treatment options. The claimed invention can therefore broaden the range of drugs available for treating diseases related to or accompanied by impaired organ or tissue function.

In one embodiment, the device for producing the claimed medicament (FIG. 1) comprises unit 1 used to record biopotentials from a donor organ or tissue (organ/tissue biopotential recording unit 1), which incorporates at least one channel amplifier 2, the inputs of which are connected to recording electrodes 3 and 4, which pick up bioelectric potentials from different sites on the donor organ or tissue, and which can be available in different designs depending on the organ or type of tissue and, therefore, recording method. Furthermore, one input of channel amplifier 2 is connected to active recording electrode 3 and the other is attached to reference (zero) recording electrode 4, or both inputs of channel amplifier 2 are connected to active recording electrodes 3 (not shown in the figure). The outputs of channel amplifier 2 are attached to corrosion-resistant (preferably made of stainless steel) current electrodes 5 and 6 of energy treatment unit 7, which have been placed in container 8 which contains the drug substance or released-active form of the drug substance. The outputs of channel amplifier 2 may be linked to low band-pass filter 9 with a bandwidth of 0.5 to 35 Hz.

Alternatively, the device used to produce the claimed medicament (FIG. 2) may comprise several channel amplifiers 2, with their one input connected to active recording electrodes 3 and the other to reference (zero) electrode 4. Furthermore, the outputs of each channel amplifier 2 are connected in parallel to current electrodes 5 and 6 of energy treatment unit 7, which have been placed in container 8 which contains the drug substance or released-active form of the drug substance, enabling summing or averaging of potential differences recorded with referential derivations right inside container 8. The outputs of channel amplifier 2 may be linked to low band-pass filter 9 with a bandwidth of 0.5 to 35 Hz.

Alternatively, the device used to produce the claimed medicament (FIG. 3) may comprise several channel amplifiers 2, with their input connected to active recording electrodes 3 and the other to reference (zero) electrode 4. Furthermore, the active outputs of said channel amplifiers 2 are connected separately (or in groups) to current electrodes 5 of energy treatment unit 7 and short-circuited (joined together) zero outputs of channel amplifiers 2 are connected to zero current electrode 6, enabling summing or averaging of potential differences recorded with referential derivations right inside container 8. The outputs of channel amplifier 2 may be linked to low band-pass filter 9 with a bandwidth of 0.5 to 35 Hz.

Alternatively, the device used to produce the claimed medicament (FIG. 4) comprises organ/tissue biopotential recording unit 1, data processing unit 10 and energy treatment unit 7. Said organ/tissue biopotential recording unit 1 contains several channel amplifiers 2, with their one input attached to active recording electrodes 3 employed in the referential derivations and the other to reference (zero) recording electrode 4. Furthermore, the outputs of each channel amplifier 2 are connected separately to the inputs of respective analog-to-digital converters 11, and the digital signal outputs are connected to the inputs of programmable data recording unit 12 used to record information onto storage device 13. Data processing unit 10 comprises programmable digital signal summing unit 14—signal summer, designed as a microcomputer, the input of which receives recorded digital data from the output of respective analog-to-digital converter 11 of organ/tissue biopotential recording unit 1 and/or individual digital data files recorded onto electronic storage devices, and the output is attached to the input of digital-to-analog converter 15 of arbitrary waveform generator 16, which generates output signals in the form of voltages (potential difference) approximating the waveform of the total biopotential difference obtained for all the bioelectric potentials recorded. The analog output of arbitrary waveform generator 16, with the peak amplitude of the output signal of up to 5-10 volts, is connected to at least two current electrodes 5 and 6 of energy treatment unit 7, placed in container 8 containing the drug substance or the released-active form of drug substance. The outputs of channel amplifier 2 may be linked to low band-pass filter 9 with a bandwidth of 0 to 35 Hz.

Alternatively, the energy-treatment of the drug substance or the released-active form of the drug substance may be accomplished by exposing it to an electrical current created by a potential difference proportional in amplitude to the amplified total biopotential difference obtained for all organ or tissue recordings done from a distance using ultrasensitive magnetometers—superconducting quantum interference devices—immersed in liquid helium (see A. N. Shestakova, A. V. Butorina, A. E. Osadchy, Y. Y. Shtyrov. Magnetoencephalography, a novel human brain function mapping technique. Experimentalnaya Psikhologia [Experimental psychology] [in Russian], 2012; 5(2):119-134).

Where the claimed medicament is produced by exposing the drug substance or the released-active form of the drug substance to bioelectric potentials acquired from other organs or tissues capable of generating biopotentials (e.g. eyes, skin, gastrointestinal organs, heart, circulatory organs, or muscle), the device incorporates a suitable biopotential acquisition system which is normally used in medical practice for the purposes in question. Meanwhile the other device elements and their arrangement are as described above.

### EXAMPLE 1

The study evaluated the anxiolytic activity of the claimed medicament (Preparation 1) available in the energy-treated released-active form of Tetramethyltetraazabicyclooctandione, obtained by serially diluting a Tetramethyltetraazabicyclooctandione stock (matrix) solution by 100³⁰⁰ times in an aqueous solvent (concentration of 0.5 mg/mL) and using external mechanical treatment—shaking of each serial dilution.

The energy treatment of the released-active form of Tetramethyltetraazabicyclooctandione was carried out by exposing it to an electric current resulting from a potential difference applied to electrodes immersed in the stock (matrix) solution and proportional in amplitude to the amplified total composite signal constituted by the sum of outputs—bioelectric potential differences recorded for 20 minutes, with individual referential derivations, from the brain of 5 healthy donors using the international 10-20 electroencephalography system involving 19 recording electrodes placed uniformly over the scalp and a reference electrode located on the earlobe.

The drug efficacy was examined using standard non-clinical testing approaches—studying tested animals' open field behavior and behavior in the light/dark transition task.

To do this, 10 male albino Wistar rats were administered intragastrically with 5 mL/kg/day Preparation 1 for 5 days for 7 days.

Ten positive control rats were dosed intragasrically with a control compound (Preparation 2), obtained by serially diluting Tetramethyltetraazabicyclooctandione by 100³⁰⁰ times (equivalent to a C300 centesimal dilution) in an aqueous solvent and using external mechanical treatment—shaking of each serial dilution, at 5 mL/kg daily for 7 days.

Negative control animals received 5 mL/kg intragastric doses of control compound (Preparation 3), obtained by serially diluting distilled water by 100³⁰⁰ times (equivalent to a C300 centesimal dilution) in an aqueous solvent and using external mechanical treatment—shaking of each serial dilution, for 7 days.

Rats' behavioral responses were studied using the above tests before administering preparations 1, 2, and 3 (baseline) and on the day following the last dose of preparations 1, 2, and 3 (day 8 of the experiment).

The animals' locomotor and exploratory activity was evaluated using the open-field apparatus. The latter is a round arena (98 cm in diameter, with 31 cm high walls) divided by drawn lines into 4 central and 28 peripheral square sectors. The arena floor has 25 holes, 3 cm in diameter each. The field floor has 25 holes 3 cm in diameter. The apparatus is lit evenly from above by a 100 W lamp. For the first 5 minutes, the animals were observed for latency to enter the central sectors, the number of central squares crossed and number of rears performed by rats in the peripheral zone of the arena.

The light/dark transition test was used to examine the animals' behavior under alternating stress conditions and their exploratory motivation/anxiety balance. The apparatus consists of two equally sized (22 cm*44 cm*30 cm) compartments—a dark chamber and a brightly illuminated one. The chambers are separated by a partition with a central door (10 cm high and 9 cm wide), through which test animals can move freely between the two compartments. The animals were observed for 5 minutes to evaluate: latency time to first enter into the dark compartment, number of moves between the compartments, time spent by rats in each compartment, number of peeps through the door when on the dark side, and number of rears.

The obtained data were expressed as the mean (M) and standard error of the mean (SEM). Between-group statistics was performed using the Mann-Whitney U test. The differences in data between day 8 and baseline were analyzed for statistical significance with the Wilcoxon test. The differences were considered significant at p<0.05.

Preparation 1 was shown to reduce rats' latency to enter the central sectors by 28.5% (p<0.05) and 21% (p<0.05), respectively, as compared to baseline and control values. The number of peripheral squares crossed was reduced by 49.5% (p<0.05) and 48.9% (p<0.05), respectively, vs. baseline and Preparation 2. At the same time, the number of central squares crossed was increased by 116% (p<0.05) and 18% (p<0.05), respectively, as compared to the baseline and control data. The number of rears at the arena's periphery was reduced by 21.6% (p<0.05) compared to baseline. Negative control animals were unaffected by the administration of Preparation 3 (Table 1)

TABLE 1
Open-field behavior of male rats administered with Preparation 1

| | | Preparation 1 | Preparation 2 | Preparation 3 |
|---|---|---|---|---|
| Parameter | Conditions | (M ± SD) | (M ± SD) | (M ± SD) |
| Latency time | Baseline | 243.2 ± 13.36 | 276.4 ± 9.8 | 251 ± 3.84 |
| to enter the | Day 8 | 173.7 ± 3.40*^{#} | 220.1 ± 6.13* | 252 ± 2.56 |
| central zone, | | | | |
| s | | | | |
| Number of | Baseline | 24.6 ± 6.24 | 44.5 ± 5.34 | 43.1 ± 3.24 |
| peripheral | Day 8 | 12.4 ± 5.33*^{#} | 24.3 ± 8.34 | 43.3 ± 2.22 |
| squares | | | | |
| crossed, n | | | | |
| Number of | Baseline | 0.6 ± 0.31 | 1.1 ± 0.43 | 0.9 ± 0.21 |
| central | Day 8 | 1.3 ± 0.72* | 1.1 ± 0.23 | 0.8 ± 1.21 |
| squares | | | | |
| crossed, n | | | | |
| Number of | Baseline | 7.4 ± 2.80 | 11.2 ± 4.82 | 7.32 ± 3.92 |
| rears at the | Day 8 | 5.8 ± 4.42* | 4.5 ± 2.26* | 7.30 ± 1.34 |
| arena's | | | | |
| periphery, n | | | | |

Note:
*p < 0.05 - significant difference vs. baseline (within-group comparisons);
^{#}p < 0.05 - significant difference vs. preparation 2 (Day 8).

In the light/dark transition task, rats' latency to enter the dark compartment was increased by 48% (p<0.05) compared to baseline. On the contrary, the number of transitions between the compartments was decreased by 54.5% (p<0.05) compared to baseline. In the group of preparation 1, the time spent by tested animals in the light compartment was increased by 48.7% (p<0.05) and 34.2% (p<0.05), respectively, as compared to baseline and control values (Preparation 2). The time spent in the dark compartment was reduced by 12.3% (p<0.05) compared to baseline. There were decreases of 37.3% (p<0.05) and 48.6% (p<0.05), respectively, in the number of rears vs. baseline and control data (Preparation 2). Negative control animals were unaffected by the administration of Preparation 3 (Table 2).

TABLE 2
Light/dark transition behavior of male rats administered with the tested drug.

| | | Preparation 1 | Preparation 2 | Preparation 3 |
|---|---|---|---|---|
| Parameter | Conditions | (M ± SEM) | (M ± SD) | (M ± SEM) |
| Latency time to | Baseline | 6.3 ± 3.24 | 17.3 ± 5.2 | 10.1 ± 5.9 |
| first enter the | Day 8 | 12.3 ± 4.58* | 11.6 ± 2.45 | 10.0 ± 2.8 |
| dark compartment, s | | | | |
| Number of | Baseline | 2.64 ± 0.59 | 2.5 ± 0.66 | 2.62 ± 0.42 |
| moves between the | Day 8 | 1.2 ± 0.41* | 4.2 ± 0.78 | 2.68 ± 0.12 |
| compartments, n | | | | |
| Time spent in the | Baseline | 23.4 ± 5.57 | 54.4 ± 12.32 | 45.2 ± 8.14 |
| light compartment, s | Day 8 | 45.6 ± 3.54*^{#} | 30.0 ± 4.44 | 44.9 ± 1.34 |
| Time spent in the | Baseline | 282.5 ± 6.55 | 245.6 ± 24.43 | 267 ± 10.10 |
| dark compartment, s | Day 8 | 247.7 ± 21.73* | 239.0 ± 23.54 | 268 ± 13.67 |
| Number of peeps from the | Baseline | 6.4 ± 5.30 | 4.9 ± 4.52 | 4.0 ± 1.1 |
| dark compartment, n | Day 8 | 3.9 ± 3.67 | 6.6 ± 0.93 | 4.2 ± 3.1 |
| Number of rears, n | Baseline | 9.1 ± 4.40 | 11.6 ± 4.27 | 10.10 ± 5.0 |
| | Day 8 | 5.7 ± 3.72*^{#} | 11.1 ± 2.42 | 9.84 ± 4.7 |

Note.
*p < 0.05 vs. baseline (intra-group “before-after” comparison)

As shown by the data above, the claimed medicament has marked anxiolytic activity, and it can be used to treat impaired brain function.

### EXAMPLE 2

The study investigated the efficacy of the claimed medicament (Preparation 1) in the treatment of diabetic retinopathy. Preparation 1 was available as a 10 mg/mL Phenylephrine aqueous solution previously energy-treated by 20-min exposure to an electric current resulting from a potential difference applied to electrodes immersed in the solution and proportional in amplitude to the amplified ERG potential difference signal picked up from the retina of a healthy donor.

The study utilized the streptozotocin-induced retinal vascular injury (diabetic retinopathy) model.

Ten male albino Wistar rats were administered intragastrically with 5 mL/kg/day Preparation 1 for 5 days before and 5 days after streptozotocin challenge.

Positive control rats were dosed with Preparation 2—a 10 mg/mL Phenylephrine aqueous solution.

Ten negative control animals (Preparation 3) received intragastric 5 mL/kg/day doses of distilled water for 5 days before and 5 days after streptozotocin challenge.

Diabetic retinopathy was induced by injecting rats, which had received 30% sucrose in drinking water for 3 weeks, intraperitoneally with 25 mg/kg streptozotocin (Zanozar, Teva Parenteral medicines Inc, USA) in 0.1 M citrate buffer, pH 4.5. Ten intact (untreated) animals were used as a model reproducibility control.

Retinal function was evaluated in rats with induced diabetic retinopathy by electroretinogram recording. To do this, animals were dark-adapted for 6 hours and anesthetized using bright red light stimuli. Pupil dilation was induced using tropicamide and the cornea was irrigated frequently with a normal saline in order to maintain the baseline response and prevent keratopathy. Rats were positioned to face a light stimulus (LED) placed 20 cm away. The recording time was 20 minutes. The reference electrode was inserted into the rat ear canal, the earth electrode was attached to the tail, and the recording electrode contacted the retina's central portion. ERGs were recorded from both eyes simultaneously: 10 responses to white light flash stimuli (5 ms, 0.2 Hz) with a maximum luminance of 9 cd/m² without a filter. The ERG responses consisted of negative a-waves, which display photoreceptor function as the beginning of the late receptor potential, and positive b-waves, which represent the electrical activity of bipolar cells and Müller cells.

The ERGs were recorded in triplicate at 5-minute intervals to ensure repeatability. The values obtained from rats' either eye were averaged and used to calculate the mean (M) and standard error of the mean (SEM) for each group. The oscillatory potentials were measured using a photostimulator at 0.2 Hz and high- (300 Hz) and low-frequency (100 Hz) filters. The sum of three oscillatory potentials was used for statistical analysis.

Peak delays and peak amplitudes obtained in each group were compared.

All between-group statistics was performed using two-way ANOVA, followed by Tukey's HSD tests between pairs of groups.

The mean amplitude of a-waves, b-waves and oscillatory potentials was decreased significantly in rats with induced diabetic retinopathy as compared to intact animals (Table 3). Preparation 1 normalized the lowered values to baseline levels (no significant differences from intact animals were noted).

TABLE 3
The a-wave, b-wave and oscillatory potential amplitudes
of electroretinograms from rats with induced diabetic
retinopathy receiving Preparation 1.

| | | | oscillatory |
|---|---|---|---|
| | a-wave | b-wave | potentials |
| Group | (M ± SEM) | (M ± SEM) | (M ± SEM) |
| Intact animals | 86.2 ± 0.8 | 353.3 ± 3.5 | 126.2 ± 1.2 |
| Preparation 1 | 83.1 ± 1.4^{#} | 349.1 ± 5.7^{#} | 124.7 ± 2.0^{#} |
| Preparation 2 | 72.5 ± 0.8* | 301.8 ± 3.2* | 102.4 ± 1.3* |
| Preparation 3 | 36.3 ± 1.3*^{#} | 154.8 ± 4.2*^{#} | 61.6 ± 0.9*^{#} |

Note:
*p < 0.05 - significant difference vs. intact animals);
^{#}p < 0.05 - significant difference vs. positive control (Preparation 2).

The peak delays were identical in all groups.

Based on the data presented, the claimed medicament has marked therapeutic activity in induced diabetic retinopathy, normalizing ERG a-wave, b-wave and oscillatory potential amplitudes and, therefore, retinal function.

### EXAMPLE 3

The study was an efficacy assessment of the claimed medicament (Preparation 1) in a myopathy animal model. Preparation 1 was used as a 3.0 mg/mL aqueous solution of polyclonal antibodies to the signal recognition particles (SRP pAb) previously energy-treated by 60-min exposure to an electric current resulting from a potential difference applied to electrodes immersed in the solution and proportional in amplitude to the amplified total composite signal constituted by the sum of outputs—bioelectric potential differences recorded from normal muscles of 5 healthy donors using electromyography.

Male Chinchilla rabbits with dexamethasone-induced myopathy were used for the experiment.

Preparation 1 was administered intravenously to 10 rabbits at a dose of 5 mL/kg/day, for 5 days prior to and 5 days after dexamethasone injection. Ten rabbits in the positive control group (Preparation 2) were intravenously injected with 3.0 mg/mL SRP pAb at 5 mL/kg/day, for 5 days prior to and 5 days after dexamethasone injection.

Ten negative control animals (Preparation 3) received 5 mL/kg/day intravenous doses of distilled water (placebo), for 5 days prior to and 5 days after dexamethasone injection.

0.8 mg/kg/day dexamethasone (Banyu Pharmaceutical Co., Tokyo, Japan) was injected intramuscularly for 14 days, to induce steroid myopathy.

A Medelec MS 4 one-channel EMG machine (the UK) was used to measure motor and sensory conduction velocity in the median nerve. Invoked potentials were recorded with a bipolar needle electrode. The stimulus needle electrode was inserted beneath the test nerve and the passive electrode was placed beneath the skin at the same position, 1.5-2 cm away from the active electrode. The recording was performed 24 hours before injecting the animals with dexamethasone and at 14 days post-challenge (60 minutes after the final injection of Preparation 1)—when morphological changes were observed in the animals' skeletal muscles. Data recorded for each animal were used to calculate the mean (M) and standard error of the mean (SEM) for the groups.

All between-group differences were statistically processed using two-way ANOVA, followed by Tukey's HSD tests between pairs of groups.

Sensory velocity in the median nerve was unchanged (within the normal range) following treatment with Preparation 1, without any effects on motor conduction (Table 4).

TABLE 4
Sensory and motor conduction velocity in the median
nerve of rabbits with experimentally induced steroid
myopathy following treatment with the test compound.

Velocity, m/s (M ± SEM)

| | | |
|---|---|---|
| | 24 hours prior to | At 14 days after |
| Type | dexamethasone | dexamethasone |

Intact animals

| | | |
|---|---|---|
| sensory | 50.4 ± 4.1 | 52.7 ± 2.9 |
| motor | 67.1 ± 8.4 | 62.3 ± 7.2 |

Preparation 3

| | | |
|---|---|---|
| sensory | 53.2 ± 3.0 | 43.3 ± 3.1* |
| motor | 64.2 ± 6.0 | 63.4 ± 6.3 |

Preparation 2

| | | |
|---|---|---|
| sensory | 55.4 ± 7.0 | 32.7 ± 3.4*^{#} |
| motor | 64.2 ± 5.4 | 66.2 ± 4.1 |

Preparation 1

| | | |
|---|---|---|
| sensory | 49.4 ± 7.4 | 51.8 ± 3.2 |
| motor | 68.1 ± 5.2 | 66.8 ± 6.7 |

Note:
*p < 0.05 - significant difference vs. baseline (within-group comparison);
^{#}p < 0.05 - significant difference vs. intact animals.

As shown by the above data, the claimed medicament demonstrated a clear therapeutic activity in myopathy, by reversing altered sensory conduction velocity in the median nerve.

### EXAMPLE 4

The study examined the efficacy of the claimed medicament (Preparation 1) in chronic heart failure. Preparation 1 was used as a 1:1 v/v energy-treated mixture of the following:

A) the released-active form of polyclonal antibodies to S100 protein (S100 pAb), obtained by mixing three different serial dilutions of an S100 pAb stock (matrix solution) prepared in an aqueous solvent (concentration of 2.5 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to endothelial NO synthase (eNOS pAb), obtained by mixing three different serial dilutions of an eNOS pAb stock (matrix solution) prepared in an aqueous solvent (concentration of 1.0 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

The above mixture was energy-treated by 30-minute exposure to an electric current resulting from a potential difference applied to electrodes immersed in the stock (matrix) solution and proportional in amplitude to the amplified total composite signal constituted by the sum of outputs—bioelectric potential differences recorded from healthy circulatory organs (blood vessels) of 3 healthy donors using impedance plethysmography.

Female Wistar rats with isadrine-induced chronic heart failure were used for the experiment.

Preparation 1 was administered intragastrically to 10 rats at a dose of 5 mL/kg/day, for 5 days prior to the first injection of isadrine and for 5 days after the second injection.

Ten rats in the positive control group (Preparation 2) received, for 5 days prior to the first isadrine injection and for 5 days after the second injection, 5 mL/kg/day intragastric doses of the 1:1 v/v mixture of:

A) the released-active form of polyclonal antibodies to S100 protein (S100 pAb), obtained by mixing three different serial dilutions of an S100 pAb stock (matrix solution) prepared in an aqueous solvent (concentration of 2.5 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to endothelial NO synthase (eNOS pAb), obtained by mixing three different multiple dilutions of an eNOS pAb stock (matrix solution) prepared in an aqueous solvent (concentration of 1.0 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

Ten rats in the negative control group (Preparation 3) received, for 5 days prior to the first isadrine injection and for 5 days after the second injection, 5 mL/kg/day intragastric doses of the released-active form of distilled water, obtained by mixing three different serial dilutions of distilled water prepared in an aqueous solvent: The following dilutions were mixed at a 1:1:1 (v/v) ratio:

1) distilled water diluted by 100¹² times (or to C12).

2) distilled water diluted by 100³⁰ times (or to C30), each time in combination with external mechanical treatment (shaking).

3) distilled water diluted by 100²⁰⁰ times (or to C200), each time in combination with external mechanical treatment (shaking).

0.8 mg/kg isadrine (isoprenaline hydrochloride) (Sigma-Aldrich, the USA) was injected subcutaneously twice at a 24-hour interval, to induce chronic heart failure.

Plethysmograms used to monitor hemodynamic parameters were recorded with a 4PΓ-2M plethysmograph (Russia). Stroke volume and cardiac output were evaluated prior to the first and 7 days after the second isadrine injection.

Date recorded for each animal were used to calculate the mean (M) and standard error of the mean (SEM) in each groups. All between-group statistics was performed using the Wilcoxon test.

Rats in the positive and negative control groups developed heart failure: decreased cardiac pump function led to a reduction of 62% and 62.6%, respectively, in cardiac output and a reduction of 71% and 77%, respectively, in stroke volume (Table 5). In the group of the test drug, animals with induced chronic heart failure demonstrated restored hemodynamic parameters following test treatment.

TABLE 5
Hemodynamic parameters of rats with experimentally induced chronic
heart failure following treatment with Preparation 1.

| | | |
|---|---|---|
| | Prior to isadrine | 7 days after isadrine |
| Parameter | injection | injection |

Preparation 3

| | | |
|---|---|---|
| cardiac output, mL | 60.2 ± 5.6 | 22.5 ± 1.7* |
| (M ± SEM) | | |
| stroke volume, mL | 0.185 ± 0.047 | 0.043 ± 0.013* |
| (M ± SEM) | | |

Preparation 2

| | | |
|---|---|---|
| cardiac output, mL | 62.4 ± 5.8 | 23.7 ± 4.1* |
| (M ± SEM) | | |
| stroke volume, mL | 0.197 ± 0.017 | 0.057 ± 0.015* |
| (M ± SEM) | | |

Preparation 1

| | | |
|---|---|---|
| cardiac output, mL | 61.7 ± 7.4 | 59.8 ± 4.2^{#$} |
| (M ± SEM) | | |
| stroke volume, mL | 0.185 ± 0.019 | 0.178 ± 0.021^{#$} |
| (M ± SEM) | | |

Note:
*p < 0.05 - significant difference vs. baseline (within-group comparison);
^{#}p < 0.05 - significant difference vs. preparation 2 as observed 7 days after isadrine injection.
^{$}p < 0.05 - significant difference vs. preparation 3 as observed 7 days after isadrine injection.

The above data suggest that the claimed medicament has clear therapeutic activity in chronic heart failure, restoring altered hemodynamic parameters—cardiac pump function and cardiac output.

### EXAMPLE 5

The study examined the effects of the claimed medicament (Preparation 1) on EDA (electrodermal activity) measures obtained under stress. Preparation 1 was used as a C10 dilution of energy-treated released-active form of monoclonal antibodies to keratin (keratin mAb), obtained by mixing two different serial dilutions of a keratin mAb stock (matrix solution) prepared in an aqueous solvent (concentration of 2.5 mg/mL) The following dilutions were mixed at a 1:2 (v/v) ratio:

1) the stock (matrix) solution diluted by 10⁶ times (or to D6).

2) the stock (matrix) solution diluted by 100⁵⁰ times (or to C50), each time in combination with external mechanical treatment (shaking).

The energy treatment of the released-active form of keratine mAb was carried out by exposing it (for 60 minutes) to an electric current resulting from a potential difference applied to electrodes immersed in the stock (matrix) solution and proportional in amplitude to the amplified total composite signal constituted by the sum of outputs—bioelectric potential differences recorded from the skin of 2 healthy donors using EDA measurement under non-stress conditions.

Once energy-treated, the product was used as the stock (matrix) solution, which was diluted serially by 100¹⁰⁰ times (to C100), to obtain Preparation 1.

The effects of the test drug were studied by measuring EDA changes in Male albino Wistar rats in response to stressful stimuli.

Preparation 1 was administered intragastrically to 10 rats at a dose of 5 mL/kg/day, for 5 days prior to delivering the stimulus, with the last dose administered 60 minutes prior to testing.

Ten positive control rats (Preparation 2) received, for 5 days prior to exposure to the stimulus (with the last dose administered 60 minutes prior to testing), 5 mL/kg/day intragastric doses of the C10 dilution of released-active keratin mAb, the latter obtained by mixing two different serial dilutions of a keratin mAb stock (matrix solution) prepared in an aqueous solvent (concentration of 2.5 mg/mL). The following dilutions were mixed at a 1:2 (v/v) ratio:

1) the stock (matrix) solution diluted by 10⁶ times (or to D6).

2) the stock (matrix) solution diluted by 100⁵⁰ times (or to C50), each time in combination with external mechanical treatment (shaking).

Ten rats in the negative control group (Preparation 3) received, for 5 days prior to exposure to the stimulus (with the last dose administered 60 minutes prior to testing), 5 mL/kg/day intragastric doses of the C10 dilution of released-active distilled water, the latter obtained by mixing two different serial dilutions of distilled water prepared in an aqueous solvent (concentration of 2.5 mg/mL) The following dilutions were mixed at a 1:2 (v/v) ratio:

1) distilled water diluted by 10⁶ times (or to D6).

2) distilled water diluted by 100⁵⁰ times (or to C50), each time in combination with external mechanical treatment (shaking).

Test rats were exposed to an auditory stimulus (a hand-clap behind the animal) or kept in pairs, for 60 s, with a rat from another cage.

EDA was measured with a 3 mm insulated electrode connected to the Nihon Kohden GSR-2 electroencelograph (Japan). The electrode was attached to an approx. 2.5 cm diameter region, previously cleared of hair, on the animal's back with a self-curing acrylic resin liquid (MEND-REX, Nissin Dental Products Inc., Japan).

EDA was measured as response time shown by animals on exposure to stimuli. Data recorded for each animal were averaged and used to calculate the mean (M) and standard error of the mean (SEM) in each group.

All between-group differences were statistically processed by two-way ANOVA, followed by Tukey's HSD tests between pairs of groups.

The data obtained for positive control animals did not differ from values recorded for the intact group (Table 6). Preparation 1 decreased the reaction time in rats presented with the noise stimulus or a “stranger” rat.

TABLE 6
Stress-induced EDA changes in rats following
treatment with Preparation 1.

Reaction time (M ± SEM)¹

| Group | Auditory stimulus | Stranger rat |
|---|---|---|
| Intact animals | 1.00 | 1.00 |
| Preparation 3 | 1.21 ± 0.10 | 1.23 ± 0.20 |
| Preparation 2 | 1.12 ± 0.11 | 1.18 ± 0.15 |
| Preparation 1 | 0.64 ± 0.05*^{#$} | 0.74 ± 0.08*^{#$} |

Note:
¹calculated by dividing the pre-treatment level by the post-treatment level;
*p < 0.05 - significant difference vs. positive control;
^{$}p < 0.05 - significant difference vs. negative control,
^{#}p < 0.05 - significant difference vs. intact animals.

Based on the above data, the claimed medicament (Preparation 1) demonstrated an effect on EDA measures under stress conditions.

### EXAMPLE 6

The study examined the efficacy of the claimed medicament (Preparation 1) in functional gastrointestinal disorders. Preparation 1 was used as a 1.5 mg/mL aqueous solution of monoclonal antibodies to histamine (histamine mAb) previously energy-treated by 4-hour exposure to an electric current resulting from a potential difference applied to electrodes immersed in the solution and proportional in amplitude to the amplified total composite signal constituted by the sum of outputs—bioelectric potential differences recorded from normal GI organs of 7 healthy donors using electrogastroenterography.

The standard experimental approach for measuring histamine-induced contractions in isolated guinea-pig ileum using tension recording was employed.

For the experiment, 2 cm ileal strips were isolated from the intestine of anesthetized guinea pigs and, once mounted in Model 4000 Isolated Organ Baths (Ugo Basile S.R.L., Italy) filled with continuously gassed and warmed (at 37° C.) Tyrode's solution (pH=7.4), were attached to an isotonic strain gauge. The Tyrode's solution was changed every 4 minutes, throughout the 40-minute equilibration period and subsequent experimentation.

At the end of the equilibration period, the strips of tissue were contracted by the addition of histamine at the final concentration of 1×10⁻⁶ M and histamine-induced responses were recorded (pre-treatment values). The recordings were performed at a resting tension of 1.0 g using a digital data acquisition device and the Lab Scribe3.0™ software.

Under test conditions, Preparation 1 (500 μl in 9.5 mL of Tyrode's solution, n=10), negative control (Preparation 3), represented by distilled water (500 μl in 9.5 mL of Tyrode's solution, n=10), and positive control (Preparation 2)—a 1.5 mg/mL aqueous histamine mAb solution (500 μl in 9.5 mL of Tyrode's solution, n=10) were added to ileum tissues. After a 2-minute incubation, 1×10⁻⁶ M histamine was added and histamine-induced responses were recorded. Between the recordings, tissues were washed in buffer every 4 minutes until they returned to normal (pre-treatment) contractile behavior (after approx. 15 minutes).

Data recorded for tissues isolated from each animal were used to calculate the mean (M) and standard error of the mean (SEM) for the groups.

All between-group differences were statistically processed using two-way ANOVA, followed by Tukey's HSD tests between pairs of groups.

Preparation 1 demonstrated an antispasmodic effect of 33.1% (Table 7).

TABLE 7
Obtained ex vivo values of histamine-induced ileal contractions
(absolute, delta mV) following treatment with Preparation 1

Amplitude, mV (M ± SEM)

| Group | Baseline | Post-treatment |
|---|---|---|
| Preparation 3 | 0.320 ± 0.036 | 0.314 ± 0.029 |
| Preparation 2 | 0.339 ± 0.038 | 0.250 ± 0.015* |
| Preparation 1 | 0.299 ± 0.032 | 0.200 ± 0.002*^{#} |

Note:
*p < 0.05 - significant difference vs. baseline;
^{#}p < 0.05 - significant difference vs. Preparation 2.

The above data suggest that the claimed medicament is capable of suppressing histamine-induced contractile response in isolated ileal tissue and is therefore effective in treating functional gastrointestinal disorders.

### EXAMPLE 7

Study Objective This was an efficacy and safety assessment of the claimed medicament (Preparation 1) in treating drug-resistant epilepsy.

Materials and Methods

Outpatients of either gender aged 18 to 70 years with confirmed diagnosis of epilepsy (G4) and epileptic seizures (occurrence frequency: at least 3 times a month) refractory to anticonvulsant therapy were included.

The participants received Preparation 1 as a solid dosage form (tablets) comprising lactose granules saturated with the released-active form of a stock (matrix) solution of monoclonal antibodies to S100 protein (S100 mAb), obtained by serially diluting an S100 mAb stock (matrix solution), pre-treated by energy exposure, by 100²⁰⁰ times (to C200) in an aqueous alcoholic solvent, in combination with mechanical treatment—shaking of each serial dilution. The energy treatment of the initial neutral carrier was carried out by exposing it (for 10 minutes) to an electric current resulting from a potential difference applied to electrodes immersed in the initial neutral carrier, wherein the applied potential difference is proportional in amplitude to the amplified total composite signal constituted by the sum of outputs—bioelectric potential differences recorded with individual referential derivations from 5 donors using the international 10-20 electroencephalography system, involving 19 recording electrodes placed uniformly over the scalp and a reference electrode located on the earlobe.

The test drug was administered at a dose of 2 tablets twice daily for 12 weeks.

Outcomes

A total of 11 patients, 3 men and 8 women (mean age: 37.6±17.5 years) were included.

Data on the occurrence of seizures in study subjects over a month's period are shown in Table. 8.

TABLE 8
Change in the number seizures at the end of the treatment period.

Number of seizures within a month

| Patient No. | Baseline | After treatment, at 12 weeks |
|---|---|---|
| 1 | 3 | 2 |
| 2 | 8 | 7 |
| 3 | 5 | 4 |
| 4 | 3 | 0 |
| 5 | 14 | 10 |
| 6 | 5 | 0 |
| 7 | 18 | 18 |
| 8 | 3 | 3 |
| 9 | 5 | 3 |
| 10 | 14 | 10 |
| 11 | 5 | 1 |
| Mean | 7.55 | 5.27 |

The mean number of seizures decreased was from 7.55 to 5.27.

After the treatment, the number of seizures was reduced significantly in 82% of subjects and remained unchanged in 18% (p<0.01). Two subjects had no seizures (18%) following treatment with the test drug.

The subjects reported improved emotional status, lower anxiety, and better sleep.

Contusions

The claimed medicament reduced seizure activity in patients with drug-resistant epilepsy, demonstrating effects on patients' psychoemotional disturbances.

### EXAMPLE 8

Patient K., 32 years.

Diagnosis: stage 1 hypertension with a low cardiovascular risk profile. Had no complaints.

Elevated supine blood pressure had been noted three times in the last month, with maximum level of 158/96 mm Hg. Increased BP had been recorded twice during examination by the doctor.

On examination: Conscious. The skin all over the body was normal in color, not cyanotic or edematous. BMI: 23. Chest: vesicular respiration, without abnormal lung noises. RR: 14 rpm. The cardiac area was not enlarged. The heart sounds were unchanged. No additional sounds or murmurs. BP: 142/94 mm Hg, pulse rate: 66 ppm. The abdomen was soft and painless on palpation. The liver was in close proximity to the costal margin. Kehr's sign, Murphy's sign, and Ortner's syndrome were negative. Urination was undisturbed. No dysuric problems. Urine output: 1200 mL/day. Defecation: once daily, formed stools. Neurological examination: without abnormalities.

ECG: sinus rhythm with normal QRS axis. The ECG was without abnormalities. Haematology: Hb: 144 g/L; WBC: 4.9×10⁹/. L (EO (%): 0, STAB (%): 5, Seg NEUT (%): 68, LYMPH (%): 20, MONO (%): 9). Bilirubin (total): 17.2 μmon; bilirubin (direct): 5.6; ALP: 47 U/L; ALT: 22 U/L; AST: 12 U/L; GGT: 21 U/L; cholesterol: 4.2 mmol/L; LDL: 2.1 mmol/L; CK: 54 U/L; glucose: 4.6 mmol/L; uric acid: 354 μmon. Urinalysis: without abnormalities. Abdomen and kidney ultrasound: without alterations.

The claimed medicament was administered to the patient as tablets containing a 1:1 v/v energy-treated mixture of the following:

A) the released-active form of polyclonal antibodies to S100 protein (S100 pAb), obtained by mixing three different serial dilutions of an S100 pAb stock (matrix solution) prepared in an aqueous solvent (concentration of 3.0 mg/mL) The following dilutions were mixed at a 1:1:1 (v/v) ratio: 1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to endothelial NO synthase (eNOS pAb), obtained by mixing three different serial dilutions of the eNOS pAb stock (matrix solution) prepared in an aqueous solvent (concentration of 2.0 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

The above mixture was energy-treated by 30-minute exposure to an electric current resulting from a potential difference applied to electrodes immersed in the stock (matrix) solution and proportional in amplitude to the amplified total composite signal constituted by the sum of outputs—bioelectric potential differences recorded from healthy circulatory organs (blood vessels) of 3 healthy donors using impedance plethysmography.

The test drug was administered at a dose of 2 tablets twice daily for 3 months. The patient received lifestyle change (proper diet and exercise) recommendations. His BP values were improved following the treatment. The self-monitored BP readings were maintained within the target levels from the second week onward, with the maximum BP of 132/86 mm Hg.

On examination: BP: 122/78 mm Hg, pulse rate: 60 ppm.

ECG: sinus rhythm with normal QRS axis The ECG was without abnormalities. No changes from previous ECGs.

Treatment outcome: improved.

Therefore, the claimed medicament has clear hypotensive activity, and can be used to treat impaired function of the circulatory system.

### EXAMPLE 9

The objective of this study was to assess the efficacy and safety of the claimed medicament in patients with dementia.

The claimed medicament was used as tablets containing a 1:1 energy-treated mixture of the following:

A) the released-active form of polyclonal antibodies to the C-terminal fragment of angiotensin II receptor type 1 (AT1 pAb), obtained by mixing three different serial dilutions of an AT1 pAb stock (matrix solution) prepared in an aqueous solvent (concentration of 3.5 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to endothelial NO synthase (eNOS pAb), obtained by mixing three different serial dilutions of an eNOS pAb stock (matrix solution) prepared in an aqueous solvent (concentration of 3.0 mg/mL) The following dilutions were mixed at a 1:1:1 (v/v) ratio:

1) the stock (matrix) solution diluted by 100¹² times (or to C12).

2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

The above mixture was energy-treated by 30-minute exposure to an electric current resulting from a potential difference applied to electrodes immersed in the stock (matrix) solution and proportional in amplitude to the amplified total composite signal constituted by the sum of outputs—bioelectric potential differences recorded from the normal heart of 4 healthy donors using electrocardiography.

Patients of either gender (age range: 60 to 85 years) with dementia of various types were included. The doctor assessed the participants' cognitive dysfunction using the Montreal Cognitive Assessment (MoCA) and rated their positive, negative and affective symptoms on the Brief Psychiatric Rating Scale (BPRS). The caretakers completed the Neuropsychiatric Inventory (NPI) for the subjects. Information on any concomitant medications was collected and recorded. The duration of treatment with the test drug was 3 months, during which time subjects completed two more visits. At Visit 2, the doctor collected the subjects' complaints, performed physical examination, reviewed the progress of study treatment and concomitant therapies, assessed treatment safety and compliance (by examining the number of tablets returned), and dispensed new doses sufficient until the next visit. At Visit 3, the doctor collected the subjects' complaints, performed physical examination, reviewed the progress of study treatment and concomitant therapies, assessed treatment safety and compliance (by examining the number of tablets returned), and dispensed new doses sufficient until the next visit. The doctor performed MoCA and the caretaker completed the NPI again.

The data were statistically processed by regression analysis (ANOVA), with subsequent Tukey's HSD test for proportions. Since no control group was used, an exact binomial model was utilized to estimate the frequency of event occurrence.

A total of 20 subjects (16 women and 4 men) were included. The mean age was 77.85±8.54 years.

No significant changes in cognitive dysfunction score were noted at the end of the study treatment (11.35±1.21 at baseline vs. 11.90±1.21 at 3 months) (Table 9).

The NPI score was decreased to 30.75±6.06 (p=0.024) from the baseline value of 54.05±6.06 (Table 10). As shown by the subsequent HSD test, the NPI score declined gradually throughout the study, with a statistically significant difference of 23.3 (p=0.023) noted by the end of the 3-month period (Table 11).

TABLE 9
Obtained MoCa score (cognitive function assessment)

| Visit | Mean MoCa score, (mean ± SE) | Statistics, p-value* |
|---|---|---|
| 1 | 11.35 ± 1.21 | p = 0.749 |
| 3 | 11.90 ± 1.21 | |

regression analysis (ANOVA)

TABLE 10
Obtained NPI scores (behavioral change
and psychopathology assessment)

| Visit | Mean NPI score, (mean ± SE) | Statistics, p-value* |
|---|---|---|
| 1 | 54.05 ± 6.06 | p = 0.024 |
| 2 | 40.65 ± 6.06 | |
| 3 | 30.75 ± 6.06 | |

regression analysis (ANOVA)

TABLE 11
NPI score change in the course of study treatment

| | Mean VAS scores obtained in the | |
|---|---|---|
| Visit | course of study treatment. | Statistics, p-value* |
| 2-1 | −13.4 | p = 0.269 |
| 3-1 | −23.3 | p = 0.023 |
| 3-2 | −9.9 | p = 0.484 |

regression analysis (ANOVA) followed by the Tukey's HSD test for proportions.

The test drug normalized the bahavior and mood of patients with dementia, reducing psychopathological symptoms, while producing no significant effect on cognitive function.

### EXAMPLE 10

Materials and Methods

The claimed medicament was assessed for efficacy and safety in treating lumbar sciatica due to intervertebral disc disorder, for which it was used as tablets containing an energy-treated 2.5 mg/mL aqueous solution of monoclonal antibodies to endothelial NO-synthase (eNOS mAb) diluted in an aqueous solvent to C100.

The energy treatment of the released-active form of eNOS mAb was carried out by exposing it (for 60 minutes) to an electric current resulting from a potential difference applied to electrodes immersed in the eNOS mAb solution and proportional in amplitude to the amplified total composite signal constituted by the sum of outputs—bioelectric potential differences recorded with referential derivations from normal muscles of 2 healthy donors using electromyography.

Once energy-treated, the product was used as the stock (matrix) solution which was diluted serially by 100¹⁰⁰ times (to C100) to obtain Preparation 1.

The test drug was administered at a dose of 2 tablets twice daily.

Twenty patients (age range: 28 to 54 years) diagnosed with “Spinal osteochondrosis, exacerbation. Lumbar sciatica due to intervertebral disc disorder” were included. The mean age of study subjects was 41.05±7.46 years. The gender ratio at screening was 1:1. Subjects who completed all protocol procedures were gendered as follows: 52.6% of women and 47.4% of men.

The subjects' condition was assessed using the Numerical Rating Scale (NRS-11). Percentages of subjects with decreased pain intensity were examined at 4 days and 14 days and changes in NRS-11 score as well as mean NRS-11 scores were evaluated at 14 days. The total duration of treatment with the test drug was 14 days, during which time subjects completed two more visits. At Visit 2 (Day 4), the NRS-11 was administered again and manual therapy initiated. The final visit (Visit 3, day 14) involved assessment of treatment safety and compliance, and a repeat completion of the NRS-11. Vasoprotectives (ATC code C05) and topical products for joint and muscular pain (ATC code M02) were permitted for use during the study. The first dose of the test product was administered at the trial site. All subjects were informed of the dose level, mode of administration and storage conditions to be used for the product as well as the need for reporting on it.

Discussion

Efficacy Outcomes

At 4 days (Visit 2—end of drug treatment (without manipulation)), 8 subjects (80%) had decreased pain intensity, as self-reported on the NRS-11 (a drop of at least 1 point). At the same time, only 2 subjects (20%) in the placebo group reported lower pain intensity (p<0.05). Two subjects (20%) in the group of test drug (20%) had unchanged pain intensity (vs. 6 subjects (60%) in the placebo groups). Two subjects (20%) in the placebo group reported higher pain intensity (an increase of at least 1 point on the NRS-11) (Table 12).

TABLE 12
Percentage of subjects with changed pain intensity
as rated on the NRS-11 (Visit 2).

| Product | Result | % |
|---|---|---|
| Placebo (n = 10) | Worsened | 2 (20%) |
| Test drug (n = 10) | | 0 |
| Placebo (n = 10) | Unchanged | 6 (60%) |
| Test drug (n = 10) | | 2 (20%) |
| Placebo (n = 10) | Improved | 2 (20%) |
| Test drug (n = 10) | | 8 (80%) |

The absolute mean change in NRS-11 score was significantly higher in the group of test product (2.9±1.1 vs. 0.1±0.9; p<0.01; Table 13)

TABLE 13
NRS score change from baseline in treatment groups (Visit 2)

| Group | ΔNRS-11 (M ± SD) |
|---|---|
| Test drug | 2.9 ± 1.1 |
| Placebo | 0.1 ± 0.9 |
| p | <0.01 |

So, the improvement of paint score and percentage of subjects with decreased paint intensity were significantly increased (compared to placebo) in the group of test product, as observed at 4 days.

By the final visit, pain reduction was reported by all subjects in both groups. However, the mean NRS-11 score was significantly decreased in the group of test product compared to the placebo group (1.0±0.8 vs. 1.9±1.0; p<0.05; Table 14).

TABLE 14
Mean NRS score in treatment groups (Visit 3).

| Group | NRS score (M ± SD) |
|---|---|
| Test drug | 1.0 ± 0.8 |
| Placebo | 1.9 ± 1.0 |
| p | <0.05 |

Safety

A total of 4 subjects (20%) developed adverse events.

The identified AEs (bowel disturbances in 3 subjects, 15%) were moderate and were apparently related to the use of detralex, without discontinuation of the test drug required. Two of the subjects were those receiving placebo and one was from the group of test product.

One more AE identified (a taste in the mouth) was mild and not requiring treatment discontinuation, and it was probably related to the test drug. This AE was reversed 2 days after the treatment was terminated (after the completion of the study). The subject reporting this AE was from the placebo group.

Conclusions

The test drug was effective in treating lumbar sciatica due to intervertebral disc disorder, demonstrating a good safety profile. Therefore, the claimed medicament can be used to treat impaired muscle function.

### EXAMPLE 11

Patient B., aged 24 years. Diagnosis: Cardiac arrhythmia (frequent atrial premature beats)

Complaints of disturbed cardiac function.

For two years, the patient had had episodes of irregular heartbeat/disturbed heart function. He had undergone long-term treatments with β-blockers (propranolol, atenolol), with a good effect achieved but symptoms of disturbed rhythm recurring after treatment termination.

The medical history contained no information on cardiac disorders.

On examination: average body built. Calm facial expression, Chest: vesicular respiration, without abnormal lung noises. RR: 16 rpm. The cardiac area was not enlarged. Arrhythmic heart sounds: premature heart beats. No additional sounds or murmurs. HR: 82 bpm. BP: 122/78 mm Hg, pulse rate: 82 ppm. The abdomen was soft and painless on palpation. The liver was in close proximity to the costal margin. No signs of abdominal irritation. No dysuric problems. Defecation: once daily, formed stools. Neurological examination: without abnormalities.

ECG: sinus rhythm; atrial premature beats (APB); normal QRS axis;

Twenty-four hour Holter monitoring: dominant sinus rhythm.

Frequent APB—4230 complexes, 4.5% of total contractions. Maximum pause: 1600 ins; HR max: 142 bpm; HR min: 56 bpm. No ST changes.

Haematology: Hb: 156 g/L; WBC: 4.6×109/. L (EO (%): 0, STAB (%): 3, Seg NEUT (%): 68, LYMPH (%): 20, MONO (%): 9).

Blood biochemistry: Bilirubin (total): 21.3 μmon; bilirubin (direct): 7.2; ALP: 52 U/L; ALT: 8 U/L; AST: 24 U/L; GGT: 15 U/L; cholesterol: 5.1 mmol/L; LDL: 3.0 mmol/L; glucose: 5.2 mmol/L; TSH: 2.1 IU/L.

Urinalysis: without abnormalities.

Abdomen and kidney ultrasound: without alterations.

The claimed medicament was administered to the patient as tablets containing a 1:1 v/v energy-treated mixture of the following:

A) the released-active form of polyclonal antibodies to the C-terminal fragment of angiotensin II receptor type 1 (AT1 pAb), obtained by mixing three different serial dilutions of an AT1 pAb stock (matrix solution) prepared in an aqueous solvent (concentration of 3.0 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to endothelial NO synthase (eNOS pAb), obtained by mixing three different serial dilutions of an eNOS pAb stock (matrix solution) prepared in an aqueous solvent (concentration of 3.0 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

The above mixture was energy-treated by 30-minute exposure to an electric current resulting from a potential difference applied to electrodes immersed in the stock (matrix) solution and proportional in amplitude to the amplified total composite signal constituted by the sum of outputs—bioelectric potential differences recorded from the normal heart of 3 healthy donors using electrocardiography.

The test drug was administered at a dose of 2 tablets twice daily for 3 months.

The clinical symptoms were improved at 3 months: the heart disturbances had resolved. Based on 24-hour Holter monitoring data, the APB count was decreased to 354.

When treatment with the test drug was stopped. No recurrence of symptoms was observed in the subsequent month of follow-up.

Overall, the test drug had a beneficial effect, resulting in as a clinically significant reduction in APB frequency. The claimed medicament is effective in treating cardiac arrhythmia, including atrial premature beats.

### EXAMPLE 12

Patient S., aged 65 years. Diagnosis: Controlled stage 2 essential hypertension. High cardiovascular risk profile. Stage B chronic heart failure (Class II). Grade 2 obesity.

Complaints of dyspnea during physical exercise, fatigability, and palpitation.

More than 10 years history of hypertension. The patient was on continuous treatment with Exforge 160/5. The BP was normal. Five years earlier, the patient had been diagnosed with hypercholesterolemia and prescribed to take Clestor 10 mg. The target cholesterol level had been achieved.

On examination: In satisfactory state. Height: 168 cm; weight: 94 kg, BMI: 33. Chest: vesicular respiration, without abnormal lung noises. HR: 16 bpm. Increased apex beat. The heart was enlarged on the left side. Rhythmic heart sounds. The aortic second sound was accentuated. No additional sounds or murmurs. HR: 64 bpm. BP: 138/84 mm Hg. The abdomen was soft and painless on palpation. The liver was in close proximity to the costal margin. No signs of abdominal irritation. No dysuric problems. Defecation: once daily, formed stools. Neurological examination: without abnormalities.

ECG: sinus rhythm with horizontal QRS axis; signs of left ventricular hypertrophy.

Echocardiography: No valvular abnormality, LVH (LV posterior wall thickness: 18 mm; interventricular septum: 16 mm), ejection fraction: 56%, diastolic function.

Haematology: Hb: 140 g/L; WBC: 4.9×109/. L

Biochemistry: Bilirubin (total): 24.2 μmon; bilirubin (direct): 9.1; ALP: 34 U/L; ALT: 34 U/L; AST: 26 U/L; GGT: 22 U/L; cholesterol: 4.9 mmol/L; LDL: 2.2 mmol/L; creatinine: 98 μmon; uric acid: 312 μmon; Cockroft-Gault CrCl: 88 mL/min (75 mL/1.73 m²)

Oral glucose tolerance test: fasting glucose: 4.6 mmol/L; at 2 hours: 6.2 mmol/L.

Urinalysis: moderately increased albuminuria—less than 30 mg per 24 hours.

Abdomen and kidney ultrasound: without alterations.

Six-minute walk test: 380 meters.

In addition to the standard therapy, the patient received the claimed medicament in the form of tablets containing a 1:1 v/v energy-treated mixture of the following:

A) the released-active form of polyclonal antibodies to the C-terminal fragment of angiotensin II receptor type 1 (AT1 pAb), obtained by mixing three different serial dilutions of an AT1 pAb stock (matrix solution) prepared in an aqueous solvent (concentration of 3.0 mg/mL) The following dilutions were mixed at a 1:1:1 (v/v) ratio:

1) the stock (matrix) solution diluted by 100¹² times (or to C12).

2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to endothelial NO synthase (eNOS pAb), obtained by mixing three different serial dilutions of an eNOS pAb stock (matrix solution) prepared in an aqueous solvent (concentration of 3.0 mg/mL) The following dilutions were mixed at a 1:1:1 (v/v) ratio:

1) the stock (matrix) solution diluted by 100¹² times (or to C12).

2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

The above mixture was energy-treated by 45-minute exposure to an electric current resulting from a potential difference applied to electrodes immersed in the stock (matrix) solution and proportional in amplitude to the amplified total composite signal constituted by the sum of outputs—bioelectric potential differences recorded from the normal heart of 5 healthy donors using electrocardiography.

The test drug was administered at a dose of 2 tablets twice daily for 3 months.

The clinical symptoms were improved at three months: dyspnea was reduced, while the patient demonstrated better exercise tolerance (6-minute walk test result of 520 meters (functional class 1)).

Overall, the test drug had a beneficial effect on chronic heart failure symptoms, improving the patient's functional class (Controlled stage 2 essential hypertension. High cardiovascular risk profile). The claimed medicament is effective in treating chronic heart failure.

### EXAMPLE 13

Study Objective: an efficacy and safety assessment of the claimed medicament vs. trimebutine in patients with IBS.

Design: A randomized, parallel-group trial. Outpatients of either gender aged 18 to 60 years with confirmed IBS diagnosis were included. The diagnosis was based on the ROME III Criteria (2006), such as recurrent abdominal pain or discomfort (experienced at least on 3 days within a month in the previous 3 months) associated with 2 or more of the following: (1) relieved with defecation; and/or; (2) onset associated with a change in frequency of stool; and/or; (3) onset associated with a change in form (appearance) of stool.

The study included patients with all IBS types. According to the ROME III Criteria and the Bristol Stool Scale, subjects with IBS-D had a mushy stool (type 6) or watery (type 7) stool at least 25% of the time, with possible less frequent (less than 25% of bowel movements) hard (type 1) or lumpy (type 2) stools. Subjects with IBS-C had hard (type 1) or lumpy (type 2) stools at least 25% of bowel movements, with possible less frequent (less than 25% of the time) mushy (type 6) or watery (type 7) stools. Patients with IBS-M had hard or lumpy stools alternating with mushy or watery stools at least 25% of bowel movements. The IBS-U subgroup only had insufficient stool abnormalities to be any of the above types.

Subjects had abdominal pain rated as at least 5 points on a 11-point (from 0 to 1) visual analogue scale (VAS).

Apart from the objective assessment, the Visual Analogue Scale for Irritable Bowel Syndrome (VAS-IBS) and the Irritable Bowel Syndrome Quality of Life Questionnaire (IBS-QoL) were also utilized to evaluate changes in clinical IBS symptoms in comparison with outcomes of treatment with the reference drug.

During the treatment period (12 weeks), subjects completed 4 visits to the health center, which involved physical examination, VAS-IBS score, Bristol Stool Scale questionnaire, IBS-QoL score, and recording of concomitant medications (if any) and possible adverse events.

At Visit 1 (Day 0), subjects were assigned to parallel groups (based on the doctor's prescription) to receive either the claimed medicament (Preparation 1—tablets containing an 1.5 mg/mL aqueous solution of monoclonal antibodies to tumor necrosis factor alpha (TNF-α mAb), energy-treated by 5-hour exposure to an electrical current resulting from a potential difference applied to electrodes immersed in the stock (matrix) solution and proportional in amplitude to the amplified total composite signal constituted by the sum of outputs—bioelectric potential differences recorded from normal GIT organs of 10 healthy donors using EGEG) or trimebutine (Preparation 2). In addition, assessment of subjects' eligibility, physical examination, and IBS symptom scores (Bristol Stool Scale and VAS-IBS) were performed. The participants completed the IBS-QoL questionnaire.

The final visit (Day 84±3) included all the assessments (VAS-IBS, Bristol Stool Scale, and IBS-QoL) and recording of adverse events and use of concomitant therapy.

The total observation time was 12 weeks.

Test Product, Reference Drug, Dose and Mode of Administration

Preparation 1 was administered at a dose of 2 tablets twice daily. The duration of treatment was 12 weeks.

Preparation 2 was administered at 1 tablet (200 mg) 3 times daily. The duration of treatment was 12 weeks.

Inclusion Criteria Outpatients of either gender with confirmed IBS diagnosis based on the ROME III Criteria (2006).

Patients aged 18 to 60 years inclusive. Subjects had to meet the following ROME III Criteria and Bristol Stool Scale criteria:

IBS-D patients—mushy (loose) (type 6) or watery (type 7) stools ≥25% and lumpy (type 2) or hard (type 1) stools <25% of bowel movements.

IBS-C patients—lumpy (type 2) or hard (type 1) stools ≥25% and mushy (loose) (type 6) or watery (type 7) stools <25% of bowel movements.

IBS-M patients—lumpy (type 2) or hard (type 1) stools ≥25% and mushy (loose) (type 6) or watery (type 7) stools ≥25% of bowel movements.

IBS-U patients—insufficient stool abnormalities to be any of the above types.

Patients who rated their pretreatment abdominal pain/discomfort as >50 mm (50% or 5 points) on the VAS.

Exclusion Criteria

Onset of IBS symptoms after the age of 50 years.

Acute coronary syndrome in the previous 3 months.

Cerebrovascular accident in the previous 3 months.

Existing or previous (in the previous 10 years) malignancies.

Prior history of any laparoscopy or laparotomy surgery.

Other GIT disorders, including coeliac disease, except IBS.

Patients who have changed their smoking habit in the previous month or are planning to during the study.

Pregnancy, breast-feeding, or failure to use adequate birth control during the study and for 30 days after the last dose of the study drug.

Patients allergic to/intolerant of any constituent of the medications used in the treatment, including those with lactose intolerance.

Patients with drug abuse problems, alcoholism, or mental disorder.

Participation in other clinical trials within a month prior to entering this trial.

Patients with active tuberculosis or viral B or C hepatitis, or prior history of HIV infection.

Any comorbidity which, in the opinion of the investigator, may affect the patient's participation in the trial.

Patients on the following concomitant medications:
gastrointestinal agents (laxatives, including herbal ones, prokinetics, antispasmodics, opioid receptor agonists (e.g. loperamide, etc.); antiflatulents (e.g. espumisan, etc.);
antibiotic;
non-steroidal anti-inflammatory drugs (except analgesics taken to relieve pain not related to IBS, but not more frequently that twice a week);
psychoactive drugs (antidepressants, anxiolytics, antipsychotics, lithium compounds and sedatives);
probiotics;
other medications the PIL of which contains a statement of effects on bowel function.

Efficacy Criteria

Primary Endpoint:

Percentage of subjects achieving a reduction of at least 30% in their pain/discomfort VAS score as observed at 12 weeks.

Secondary Endpoints:

Percentage of IBS-D subjects whose Bristol Scale stool pattern has changed to at least type 5 (approx. within a week).

Change in stool type on the Bristol Stool Scale.

Percentage of IBS-C whose defecation rate has increased by approx. 1 time a week as compared to baseline.

Change in defecation rate demonstrated by IBS-C patients

Change in VAS-IBS score

Change in IBS-QOL total score.

Safety Criteria

Occurrence, nature, and duration of adverse events and AE relationship to the study treatment; AE severity and other characteristics (for identified AEs or serious AEs, a dedicated report form had to be completed for further notification of the manufacturer's clinical safety surveillance unit.

Efficacy Outcomes

All subjects were assigned to either of the treatment groups as follows:

74 subjects received Preparation 1 and

75 subjects were treated with Preparation 2.

The male to female ratio in study subjects was approximately 1 to 2, which is consistent with the findings that twice as many women as men are affected by IBS. The mean age was the same in both groups (32.7±2.7) years.

Based on the prevalent presentation of stools in IBS, all subjects were considered as follows: 72 IBS-D subjects (48.3%), 52 IBS-C subjects (34.9%), 11 IBS-M (7.4%), and 14 IBS-U (9.4%) subjects.

Effects on Pain Symptoms.

At baseline (Visit 1, Day 0), mean pain VAS scores did not differ significantly between the groups.

Overall, clear beneficial changes (p<0.01) in IBS pain scores were observed with both drugs throughout the observation period, with a trend towards a somewhat better effect with Preparation 1 (Table 15).

TABLE 15
Mean VAS scores obtained in the course of study treatment.

| | | After treatment, | |
|---|---|---|---|
| Group | Baseline | at 12 weeks | p |
| Preparation 1, M ± σ | 8.4 ± 1.7 | 3.0 ± 1.9 | p < 0.01 |
| Preparation 2, M ± σ | 8.2 ± 1.8 | 3.1 ± 2.2 | p < 0.01 |

By the end of the observation (and treatment) period, the effect of Preparation 1 was significantly increased compared to the comparator treatment (Preparation 2): a pain score reduction of at least 30% was achieved by 95% of subjects in the group of Preparation 1 vs. 83% in Preparation 2 group (p=0.0001).

By the end of the 3-month treatment period, abdominal pain was relieved in all subjects receiving the claimed drug, with the pain reduction being clinically significant and more pronounced that in the trimebutine group.

2. Effects on Stool Pattern

The analysis of changes in Bristol Stool Scale types showed that Preparation 1 was substantially more effective (compared to Preparation 2) in normalizing stool types in IBS-D: 90% of IBS-D subjects in the group of Preparation 1 demonstrated normal stool type by the end of treatment (vs. 58% in the group of Preparation 2 (p=0.0024)).

Similarly, Preparation 1 had a significantly stronger effect, compared to Preparation 2, in normalizing Bristol Stool Scale types in IBS-C subjects (p=0.0001).

Although both drugs proved effective in improving stool patterns, normal stool type was demonstrated by all study subjects only in the group of Preparation 1, whereas this variable was 1/4 lower (76% of subjects) in the group treated with Preparation 2 (p=0.0135).

The mean defecation rate demonstrated by IBS-C subjects was also indicative of a greater effect of Preparation: 4.7±0.8 times a week vs. 3.1±0.7 times a week with Preparation 2 (p=0.0001).

3. Effects on Subjects' Quality of Life

The improvement of clinical symptoms as a result of study treatment was supported by changes obtained in mean IBS-QoL score. The ANOVA results showed that the mean IBS-QoL score changes were largely comparable between the groups (Table 16)

TABLE 16
IBS-QOL total scores obtained in the course of treatment.

| Group | Baseline | After treatment, at 12 weeks | p |
|---|---|---|---|
| Preparation 1 | 96.4 ± 25.7 | 49.9 ± 24.3 | p < 0.01 |
| Preparation 2 | 94.2 ± 23.8 | 51.2 ± 20.7 | p < 0.01 |

As shown above, the test drugs were comparable in improving the quality of life of study subjects with IBS.

4. Overlap Syndrome.

Apart from IBS symptoms, manifestations of functional dyspepsia (FD, sensations of an overfilled epigastrium and nausea after eating) were recorded for almost a third of subjects in the group of Preparation 1 (n=21, 28.4%). This combination, known as an overlap syndrome, is common in functional gastrointestinal disorders.

Subjects receiving Preparation 1 appeared less concerned about abdominal discomfort at the end of the study. At 12 weeks, the unpleasant epigastric sensations were decreased by 45%, as compared to baseline (p<0.001).

The occurrence of nausea was reduced by 82% compared to the pre-treatment level (p<0.05).

Therefore, the claimed medicament also proved effective in other functional gastrointestinal disorders as well as overlap syndrome.

Safety Outcomes No unfavorable effects on the vital signs, including HR and BP, or adverse events were observed in either group during the study treatment.

The study drugs were well-tolerated and, therefore (given the efficacy observed) ensuring good treatment compliance.

Conclusion

Based on the data obtained, with efficacy and safety similar to the comparator treatment, the claimed medicament proved effective in IBS, demonstrating its therapeutic effect by greater reduction of pain (compared to trimebutine)—the main clinical symptom in this type of pathology. The claimed medicament improved subjects' stool patterns and led to better quality of life outcomes.

Additionally, the claimed medicament was shown effective in relieving FD symptoms, as well as overlap syndrome, which, together with high tolerability demonstrated, ensures good treatment compliance. So the claimed medicament is effective in treating the above functional gastrointestinal impairments.

### EXAMPLE 14

Woman S., aged 46 years Diagnosis: Raynaud syndrome. Angiospastic disease.

Complaints of cold in the fingers and toes, the latter turning painful and pale in response to cold.

The above complains had persisted for 5 years, with no effect from conventional treatment; surgical intervention (sympathectomy) was planned.

On examination: Conscious. The skin all over the body was normal in color, not cyanotic or edematous. Distal finger phalanges turned white on washing the hands in cold water. Positive cold test.

Chest: vesicular respiration, without abnormal lung noises. RR: 16 rpm. The cardiac area was not enlarged. The heart sounds were unchanged. No additional sounds or murmurs. BP: 132/78 mm Hg, pulse rate: 88 ppm. The abdomen was soft and painless on palpation. The liver was in close proximity to the costal margin. Urination was undisturbed. No dysuric problems. Neurological examination: without abnormalities.

ECG: sinus rhythm with normal QRS axis The ECG was without abnormalities

Haematology: Hb: 132 g/L; WBC: 5.1×109/. L (EO (%): 1, STAB (%): 5, Seg NEUT (%): 65, LYMPH (%): 21, MONO (%):12). Bilirubin (total): 14.3 μmon; bilirubin (direct): 5.4; ALP: 34 U/L; ALT: 19.4 U/L; AST: 17.2 U/L; GGT: 15.0 U/L; cholesterol: 4.6 mmol/L; LDH: 67 U/L; CK: 89 U/L; glucose: 4.8 mmol/L; uric acid: 312 μmon.

The test drug was administered in the form of tablets containing a 2:1 v/v energy-treated mixture of the following:

A) the released-active form of polyclonal antibodies to S100 protein (S100 pAb), obtained by mixing three different serial dilutions of an S100 pAb stock (matrix solution) prepared in an aqueous solvent (concentration of 3.0 mg/mL) The following dilutions were mixed at a 1:2:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C50), each time in combination with external mechanical treatment (shaking).

B) the released-active form of polyclonal antibodies to endothelial NO synthase (eNOS pAb), obtained by mixing three different serial dilutions of an eNOS pAb stock (matrix solution) prepared in an aqueous solvent (concentration of 3.0 mg/mL). The following dilutions were mixed at a 1:1:2 (v/v) ratio:

4) the stock (matrix) solution diluted by 100¹⁰ times (or to C12). 5) the stock (matrix) solution diluted by 100²⁰ times (or to C20), each time in combination with external mechanical treatment (shaking).

6) the stock (matrix) solution diluted by 100⁸⁰ times (or to C80), each time in combination with external mechanical treatment (shaking).

The above mixture was energy-treated by 60-minute exposure to an electric current resulting from a potential difference applied to electrodes immersed in the stock (matrix) solution and proportional in amplitude to the amplified total composite signal constituted by the sum of outputs—bioelectric potential differences recorded from normal circulatory organs (blood vessels) of 5 healthy donors using impedance plethysmography.

The test drug was taken at a dose of 2 tablets twice daily.

Improvement of clinical symptoms was recorded after three months of treatment initiation. The sensation of cold in the hands was reduced and pain response to cold was reversed.

Doubtful cold-test result.

Surgical intervention was postponed.

Treatment outcome: improved.

In summary, the claimed medicament was effective in treating Raynaud syndrome.

### EXAMPLE 15

The efficacy and safety of the claimed medicament was studied in patients with cerebral palsy.

The claimed medicament (Preparation 1) was available as tablets containing a 1:1 v/v energy-treated mixture of the following:

A) the released-active form of polyclonal antibodies to S100 protein (S100 pAb), obtained by mixing three different serial dilutions of an S100 pAb stock (matrix solution) prepared in an aqueous solvent (concentration of 3.0 mg/mL) The following dilutions were mixed at a 1:1:1 (v/v) ratio:

1) the stock (matrix) solution diluted by 100¹² times (or to C12). 2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to interferon gamma (IFNγ pAb), obtained by mixing three different serial dilutions of an IFNγ pAb stock (matrix solution) prepared in an aqueous solvent (concentration of 3.0 mg/mL) The following dilutions were used in the 1:1:1 (v/v) mixture:

4) the stock (matrix) solution diluted by 100¹² times (or to C12). 5) the stock (matrix) solution diluted by 100³⁰ times (or to C30), each time in combination with external mechanical treatment (shaking). 6) the stock (matrix) solution diluted by 100²⁰⁰ times (or to C200), each time in combination with external mechanical treatment (shaking).

The above mixture was energy-treated by 10-minute exposure to an electric current resulting from a potential difference applied to electrodes immersed in the stock (matrix) solution and proportional in amplitude to the amplified total composite signal constituted by the sum of outputs—bioelectric potential differences recorded from the brain of 5 healthy donors using electroencephalography.

Children, aged between 3 and 14 years, with motor and pseudobulbar disturbances due to cerebral palsy (ICD 10 codes: G 80.0, G 80.1, and G 80.2), who had been admitted to the Theoretical and Practical Research Center for Children's Psychoneurology (Russia), were included.

The subjects were assigned to receive Preparation 1 at a dose of 10 drops 6 times daily for 7 days, followed by 10 drops taken 3 times daily. The total study duration was 8 weeks.

Motor function impairment was assessed in study subjects at 4 weeks and 8 weeks using the GMFM-66 (Gross Motor Function Measure) inventory.

In the first 4 weeks, Preparation 1 was administered in combination with a wide range of rehabilitation interventions, including physiotherapeutic procedures, speech therapy, therapeutic exercise, and massage. In the subsequent weeks, subjects were discharged home to continue taking Preparation 1 as a standalone treatment.

The data were processed statistically using one-way ANOVA, the baseline GMFM total core (Day 0) being used as the covariate.

Results: The participants were 20 subjects (16 boys and 4 girls) suffering from different types of cerebral palsy including spastic diplegic, dyskinetic, ataxic, and hemiplegic cerebral palsies. The mean age of study subjects was 3.18±0.61 years.

The outcomes of treatment are shown in Table 17.

TABLE 17
Treatment outcomes.

| | | GMFM | GMFM | |
|---|---|---|---|---|
| | | after 4 | after 8 | |
| Subject | Baseline | weeks of | weeks of | |
| ID # | GMFM | treatment | treatment | Self-reported |
| 001 | 25 | 29 | 32 | Spastic diplegic cerebral palsy. Improved |
| | | | | muscle tone and sitting position; the child is |
| | | | | calmer and better-adapted to new environment. |
| 002 | 117 | 141 | 152 | Spastic diplegic cerebral palsy. Improved |
| | | | | muscle tone, greater range of movement in |
| | | | | some joints, reduced ataxia, improved fine |
| | | | | motor skills, better verbal comprehension, |
| | | | | enlarged vocabulary, better attention switching |
| 003 | 133 | 137 | 138 | Ataxic cerebral palsy, epilepsy. Improved |
| | | | | physical strength, and reduced pseudobulbar |
| | | | | symptoms; the child is better-adapted to new |
| | | | | environment and is less irritable. |
| 004 | 130 | 138 | 146 | Hemiplegic cerebral palsy. Improved muscle |
| | | | | tone, greater range of movement in some |
| | | | | joints, better ability to maintain focus and |
| | | | | adapt to new environment. Enlarged |
| | | | | vocabulary. |
| 005 | 50 | 60 | 60 | Spastic diplegic cerebral palsy. Improved |
| | | | | muscle tone, less joint stiffness, reduced ataxia |
| | | | | and propulsion during walking. Feels more |
| | | | | confident when walking, able to walk longer |
| | | | | distances. |
| 006 | 125 | 131 | 132 | Spastic diplegic cerebral palsy. Well-adapted |
| | | | | to the environment, improved muscle tone, |
| | | | | slightly reduced dysphonia. |
| 007 | 146 | 152 | 165 | Spastic diplegic cerebral palsy. Improved |
| | | | | muscle tone, greater range of movement in |
| | | | | some joints, better verbal comprehension, |
| | | | | enlarged vocabulary. |
| 008 | 84 | 117 | 123 | Spastic diplegic cerebral palsy. Unchanged. |
| 009 | 88 | 95 | 101 | Spastic diplegic cerebral palsy. Reduced |
| | | | | hypersalivation, anxiolythic effect |
| 010 | 60 | 72 | 86 | Spastic diplegic cerebral palsy. Improved |
| | | | | orthopedic tests, muscle tone, better verbal |
| | | | | comprehension |
| 011 | 128 | 134 | 141 | Spastic diplegic cerebral palsy. Left parietal |
| | | | | venous angioma. |
| 012 | 42 | 56 | 61 | Spastic diplegic cerebral palsy. Unchanged. |
| 013 | 42 | 56 | 61 | Spastic diplegic cerebral palsy. Normalized |
| | | | | behavior. |
| 014 | 125 | 131 | 136 | Left-sided hemiparesis Unchanged. |
| 015 | 158 | 164 | 166 | Left-sided hemiparesis Reduced ataxia during |
| | | | | walking |
| 016 | 131 | 142 | 144 | Spastic diplegic cerebral palsy. Unchanged. |
| 017 | 77 | 87 | 87 | Spastic diplegic cerebral palsy. Unchanged. |
| 018 | 17 | 20 | 25 | Spastic diplegic cerebral palsy. Unchanged |
| 019 | 95 | 101 | 108 | Spastic diplegic cerebral palsy. Unchanged |
| 020 | 35 | 52 | 57 | Ataxic cerebral palsy. Epilepsy. Unchanged. |

On the whole, improvement of muscle tone and increased joint movements were mainly recorded, with two subjects (10%) demonstrating reduced pseudobulbar symptoms. The majority of subjects showed better environmental adaptation, verbal production and verbal comprehension.

The GMFM-66 mean motor function score was increased by 11.2±8.3 and 16.4±9.6, respectively, at Visit 2 and Visit 3. Preparation 1 also had a positive effect when taken in home settings (after subjects were discharged from hospital): the improvement achieved during inpatient treatment was maintained and even increased (Table 18).

In addition, changes in item scores (Item A: Lying and Rolling Score; Item B: Sitting Score; Item C: Crawling and Kneeling Score; Item D: Standing Score; and Item E: Walking, Running and Jumping Score) were evaluated.

TABLE 18
GMFM-66 Total Score changes.

| | Days | | | | | |
|---|---|---|---|---|---|---|
| | Day 0 | | Week 4 | | Week 12 | |
| | GMFM | | GMFM | | GMFM | |
| | Total | % | Total | % | Total | % |
| Variable | Score | Change | Score | Change* | Score | Change* |
| Mean | 95.3 | 0 | 106.5 | 11.2 | 111.7 | 16.4 |
| SD | 45.1 | 0 | 46.1 | 8.3 | 45.7 | 9.6 |
| Min | 17 | 0 | 20 | 3 | 25 | 5 |
| Max | 158 | 0 | 177 | 31 | 178 | 39 |

Based on the above data, treatment with Preparation 1 has a beneficial effect on patients with cerebral palsy, reducing muscle tone, increasing range of joint movement, and ameliorating pseudobulbar symptoms. It improves patients' environmental adaptation, verbal production and verbal comprehension.

So the claimed medicament is effective in treating cerebral palsy and developmental delay.

### EXAMPLE 16

The test drug (Preparation 1) was used as an aqueous diclofenac sodium solution previously energy-treated by 6-hour exposure to an electric current resulting from a potential difference applied to electrodes immersed in the solution and proportional in amplitude to the amplified total composite EGEG signal constituted by the sum of outputs—bioelectric potential differences recorded from normal GIT organs of seven healthy donors.

The experimental acute toxicity study of diclofenac sodium (Preparation 2) and the claimed medicament (Preparation 1) utilized male outbred CD-1 mice, aged 8 weeks (weight: 20 to 22 g; n=45).

Preparation 2 was LD50 dose of 53 mg/kg. Similarly, Preparation 1 was administered intragastrically at a single dose calculated as the maximum volume allowable for intragastric administration according to the “Guidance on experimental (pre-clinical) investigation of novel pharmacological agents” (2012), i.e. 0.5 mL/mouse. Control mice were intragastrically dosed with a single 0.5 mL/mouse dose of purified water (Preparation 3).

Observations of study animals were performed for 14 weeks after dose administration, including daily physical examination, weight measurements (prior to dose administration, and on days 8 and 15 thereafter), and postmortem examination (gross and microscopic changes at the end of the experiment).

Data recorded for each animal were used to calculate the mean (M) and standard error of the mean (SEM) in each group.

Between-group statistics was analyzed by one-way ANOVA coupled with Tukey's HSD test and Kaplan-Meier curves, followed by log-rank tests between pairs of groups. Obtained differences were statistically significant at p<0.05.

Mice dosed with Preparation 2 demonstrated decreased motor activity within 2 hours of dose administration. On day 7, the animals had rumpled hair coat, were hypodynamic and debilitated, and had bloating and melena. On day 15, surviving mice showed weight loss (Table 19). Earliest death cases were recorded in study mice 3 days after the administration of Preparation 2 (Table 20). At the same time, no differences from the control mice were revealed on physical examination in the group of Preparation 1, nor were any deaths recorded in this group.

TABLE 19
Weights of male mice (g) after receiving
single doses of Preparation 1, M ± SEM

| Group | Before dosing | Day 8 | Day 15 |
|---|---|---|---|
| Preparation 3 (n = 15) | 21.80 ± 0.94 | 23.46 ± 0.81 | 24.02 ± 0.78 |
| Preparation 2 (n = 15) | 21.36 ± 0.64 | 19.98 ± 1.46 | 14.42 ± 1.53* |
| Preparation 1 (n = 15) | 20.50 ± 0.97 | 21.34 ± 1.22 | 23.82 ± 0.74 |

Note:
*p < 0.05 vs control.

TABLE 20
Survival rates in the groups of male mice (g) following the intragastric
dose of diclofenac or MMH's sample, M ± SEM

| | Day of the experiment | | | | | | | | | | | | | | |
|---|---|---|---|---|---|---|---|---|---|---|---|---|---|---|---|
| Group | 1 | 2 | 3 | 4 | 5 | 6 | 7 | 8 | 9 | 10 | 11 | 12 | 13 | 4 | 15 |
| Preparation 3 | 0/15 | 0/15 | 0/15 | 0/15 | 0/15 | 0/15 | 0/15 | 0/15 | 0/15 | 0/15 | 0/15 | 0/15 | 0/15 | 0/15 | 0/15 |
| Preparation 2 | 0/15 | 0/15 | 4/15 | 2/11 | 3/9 | 0/6 | 0/6 | 0/6 | 0/6 | 0/6 | 0/6 | 0/6 | 0/6 | 0/6 | 0/6 |
| Preparation 1 | 0/15 | 0/15 | 0/15 | 0/15 | 0/15 | 0/15 | 1/15 | 0/14 | 0/14 | 0/14 | 0/14 | 0/14 | 0/14 | 1/14 | 0/13 |

Note:
presented as ‘number of dead animals’/‘number of animals in the group’

The postmortem examination revealed right heart and visceral congestion in mice found dead on day 3 and day 4 (in response to declofenac sodium). These mice had anemic brain and gastrointestinal mucosa. The microscopic examination of major organs demonstrated congested veins and intermuscular capillaries of the myocardium, and marked interstitial edema. Signs of acute venous congestion were noted in the lungs and liver. In the kidneys, the veins and capillaries in the interstitium and nephron glomeruli were congested; swollen and vacuolated tubular epithelium, pyknotic nuclei, and individual cells desquamated into the lumen were observed. Mucosal microerosions were observed microscopically in the stomach. Animals for which death was recorded on day 5 demonstrated the presence of black intestinal contents, and acute ulceration in the stomach.

The data obtained in this study, therefore, show that the claimed medicament is non-toxic.

### EXAMPLE 17

An efficacy and safety study of the claimed medicament in patients with endogenous acute psychotic disorder (hallucinations and delusions).

The open-label, non-comparative study was conducted to examine the efficacy and safety of the claimed medicament (Preparation 1) in the treatment of acutely psychotic (endogenous etiology) patients with hallucinations and delusions.

Preparation 1 was administered as tablets containing a 1:1 v/v energy-treated mixture of the following:

A) the released-active form of polyclonal antibodies to S100 protein (S100 pAb), obtained by mixing three different serial dilutions of an S100 pAb stock (matrix solution) diluted in an aqueous solvent (concentration of 2.5 mg/mL) The following dilutions were mixed at a 1:1:1 (v/v) ratio:

1) the stock (matrix) solution diluted 100¹² by (or to C12).

2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to cannabinoid receptor type 1 (CB1 pAb), obtained by mixing three different serial dilutions of a CB1 pAb stock (matrix solution) diluted in an aqueous solvent (concentration of 3 mg/mL). The following dilutions were used in the 1:1:1 (v/v) mixture:

1) the stock (matrix) solution diluted by 100¹² times (or to C12).

2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

The above mixture was energy-treated by 30-minute exposure to an electric current resulting from a potential difference applied to electrodes immersed in the stock (matrix) solution and proportional in amplitude to the amplified total composite signal constituted by the sum of outputs—bioelectric potential differences recorded from the healthy circulatory organs (blood vessels) of 3 healthy donors using impedance plethysmography.

Study Design

Inpatients in an acute episode of psychotic disorder requiring anti-psychotic therapy were included. At visit 1, the doctor assessed the psychopathological symptoms including affective (psychomotor agitation) and productive (hallucinations/delusions) symptoms and completed the BPRS. The subjects were assigned to study treatments. Further mental assessment, including the BPRS, was performed after 28 days.

Efficacy Criteria

BPRS score change at 28 days.

Safety Criteria

Occurrence/nature of adverse events during the therapy and AE relationship to the study treatment.

Test Product, Dose and Mode of Administration

Preparation 1 was administered orally (a dose of 20 drops) 4 times daily over a period of 28 days.

Duration of Observation

A 28-days observation period.

Concomitant Medications

Preparation 1 was used concomitantly with previously prescribed anti-psychotic medications (neuroleptics including: haloperidol, aminazine, azaleptin, triftazin, tisercin, moditen depo, serdolect, torendo, and trihexyphenidyl (neurolepsy corrector). The main criterion for inclusion was patients on a sufficiently lengthy prior therapy (unchanged for at least 30 days) whose condition had been stable on addition of Preparation 1 for over 14 days. No other psychoactive drugs were permitted during the study.

Outcomes

A total of 38 subjects, 16 men and 22 women, (mean age: 47.6±16.2 years) were included.

All subjects were inpatients who had been admitted to a psychiatric department with an acute psychotic episode and who required anti-psychotic therapy.

The diagnoses on admission were:

1) ‘Schizophrenia, paranoid type, paranoid syndrome. Continuous course with progressive deficits’ (27 subjects (71%));

2) ‘Organic brain disease of complex genesis (toxic, vascular), hallucinatory-paranoid syndrome’ (11 subjects (29%).

All subjects had been using neuroleptics on inclusion in the study.

The efficacy analysis using the BPRS inventory revealed a significant (p<0.0001) reduction in the severity of psychotic symptoms 28 days after the first dose of the test drug (Table 21)

TABLE 21
BPRS psychopathalogical symptom
scores in the course of treatment

| | Total BPRS score | |
|---|---|---|
| Scoring | N = 38 | Statistics |
| Baseline | 69.2 ± 9.8 | p < 0.0001 |
| Day 28 | 27.1 ± 8.5 | |

As judged by the doctors, study subjects receiving anti-psychotic medications in addition to the standard therapy demonstrated better mental state improvement as compared to historical control data (disease progress data from previous hospitalizations).

Additionally, neurolepsy symptoms were significantly reduced in subjects treated with conventional neuroleptics.

No adverse events related to Preparation 1 were developed by any of the subjects.

Therefore, the addition of the claimed medicament to treatment with the above neuroleptics led to a noticeable mental state improvement and prevented the development of neuroleptic complications in study subjects.

### EXAMPLE 18

The study determined the effectiveness of the effect of the claimed medicament (Preparation 1) on the sleep-wake cycle. Preparation 1 is an energetically processed release-active form of polyclonal antibodies to the type 1 cannabinoid receptor.

Preparation 1 was obtained in the following way. The original substance of antibodies to cannabinoid receptor type 1 (anti-CB1, 3 mg/ml) is diluted 100 times with 15% ethanol, after which energy treatment of the resulting dilution is carried out for 1 hour by passing an electric current under the action of a potential difference applied electrodes placed in solution, which is proportional in amplitude to the amplified combined total signal formed by adding the resultant signals—the differences in the bioelectric potentials of the human brain recorded in n five healthy donors using electroencephalography (EEG). Then, the resulting dilution, treated with the transformed biopotentials of the human brain, is diluted by successively reducing the initial concentration to obtain a C200 dilution in combination with an external mechanical effect—shaking each dilution.

Brain biopotential transformations are performed using a special computer program that processes electroencephalograms of healthy volunteers of different sexes and ages, not showing signs of abnormalities, recorded in accordance with the recommendations of the International Federation of Clinical Neurophysiology, until a single, unified (averaged) signal is obtained.

White outbred male rats (220-250 g) were used in the study. Preparation 1 was administered to 5 rats once intraperitoneally at a dose of 5 ml/kg/day. In the control group (Preparation 2), purified water was administered to 5 rats once intraperitoneally at a dose of 5 ml/kg/day.

For 5-6 days, an animal anesthetized with sodium etamininal (50 mg/kg intraperitoneally) was preformed using a stereotactic device to implant electrodes into the sensorimotor cortex (AP: −1; L: 1.5; H: 1.5), the lateral zone of the hypothalamus (AP: 2; L: 1-1.5; H: 8-8.5) and the dorsal section of the hippocampus (AP: 3; L: 2; H: 3.3-3.5). The indifferent electrode used in monopolar recording was implanted into the nasal bone of the skull. The grounding electrode was implanted into the occipital part of the skull.

Registration of the electrical activity of the brain was carried out under conditions of free behavior of rats in an experimental chamber using the Neuro-KM hardware-software complex (Statokin, Moscow). After twice a 10-minute recording of the background EEG, the test drugs were intraperitoneally injected into the animals without stopping the recording of the EEG and then the EEG was recorded for 2 hours. In this case, due to the long stay of the rats in the experimental setup, most of the experiment was recorded in sleeping animals, i.e. in a state of sleep, which is characterized by a sharp change in the wave activity of the brain compared with the state of wakefulness and the predominance of delta and theta rhythms in various stages of sleep.

Estimation of the degree of change in the bioelectrical activity of the rat brain for all physiologically significant frequency ranges (delta, theta, alpha, beta1 and beta2 waves) was carried out by comparing the EEG (spectral analysis) at different times after the administration of drugs relative to the background values individually for every animal.

Computer and statistical analysis of spectral-coherent characteristics was performed using the program “BRAINSYS”.

The claimed medicament (Preparation 1) increased (p>0.05) quantitative indicators of slow-wave and fast-wave sleep, reduced the duration of wakefulness, and also increased (p<0.05) the number of wakefulness episodes. In addition, Preparation 1 caused unidirectional and pronounced changes in the spectral power of the EEG in the cortex, hippocampus and hypothalamus in all animals, which resulted in a decrease in the slow-wave part of the spectrum delta and low-wave theta activity—and an increase in the fast-wave part of the spectrum—alpha, beta1 and beta2 activity during the 2-hour observation period (Table 22).

TABLE 22
The effect of Preparation 1 on the change of sleep-wake cycles in rats during 2 hours of EEG recording

| | | | Number of | | | | Duration (T), | | | | | |
|---|---|---|---|---|---|---|---|---|---|---|---|---|
| Terms of | LP, min | | episodes | | | Tₜₒₜₐₗ, | min | | | Tₛₛ/Tₜₒₜₐₗ * | Tₛₛ/Tₜₒₜₐₗ * | T_{WF.}/ |
| experience | SS | FS | SS | FS | WF | min | SS | FS | WF | 100, % | 100, % | Tₜₒₜₐₗ *100, % |
| Background | 39.0 ± | 87.1 ± | 11.6 ± | 3.8 ± | 12.2 ± | 129.2 ± | 64.6 ± | 7.1 ± | 58.8 ± | 49.3 ± | 9.5 ± | 45.2 ± |
| Indicators | 19.2 | 34.3 | 2.5 | 1.5 | 2.9 | 3.4 | 4.5 | 2.4 | 7.5 | 4.5 | 4.9 | 5.6 |
| Preparation | 26.9 ± | 73.5 ± | 10.2 ± | 4.0 ± | 10.6 ± | 125.4 ± | 59.0 ± | 6.5 ± | 59.9 ± | 47.1 ± | 5.2 ± | 47.7 ± |
| 1 | 6.2 | 19.4 | 2.1 | 1.5 | 2.0 | 9.3 | 7.1 | 2.3 | 9.3 | 4.2 | 1.7 | 5.7 |
| Paired | P = | P = | P = | P = | P = | P = | P = | P = | P = | P = | P = | P = |
| Student t- | 0.597 | 0.572 | 0.135 | 0.906 | 0.242 | 0.681 | 0.581 | 0.867 | 0.904 | 0.714 | 0.497 | 0.729 |
| test | | | | | | | | | | | | |
| Background | 33.5 ± | 83.2 ± | 8.6 ± | 2.8 ± | 8.8 ± | 125.7 ± | 59.9 ± | 3.6 ± | 53.9 ± | 48.8 ± | 3.2 ± | 44.0 ± |
| Indicators | 7.1 | 15.2 | 1.1 | 0.8 | 1.0 | 3.6 | 8.8 | 1.4 | 10.7 | 7.8 | 1.2 | 7.5 |
| Preparation | 23.0 ± | 56.7 ± | 13.8 ± | 5.0 ± | 14.4 ± | 125.0 ± | 66.7 ± | 10.0 ± | 48.3 ± | 53.6 ± | 7.9 ± | 38.5 ± |
| 2 | 7.1 | 10.6 | 2.3 | 0.8 | 2.3 | 4.3 | 5.2 | 3.1 | 8.7 | 4.3 | 2.3 | 6.4 |
| Paired | P = | P = | P = | P = | P = | P = | P = | P = | P = | P = | P = | P = |
| Student t- | 0.413 | 0.354 | 0.073 | 0.180 | 0.050 | 0.879 | 0.635 | 0.179 | 0.781 | 0.712 | 0.202 | 0.708 |
| test | | | | | | | | | | | | |

Note:
data are presented as M ± SEM.
LP—latent period, T—duration, SS—slow sleep, FS—fast sleep, WF—wakefulness.

Thus, it is shown that the claimed medicinal product (Preparation 1) affects the spectral power of the EEG and the sleep-wake cycle of experimental animals.

### EXAMPLE 19

The study determined the anxiolytic activity of the claimed medicinal product (Preparation 1). Preparation 1 is an energetically processed release-active form of polyclonal antibodies to the type 1 cannabinoid receptor.

Preparation 1 was obtained in the following way. The original substance of antibodies to cannabinoid receptor type 1 (anti-CB1, 5 mg/ml) is diluted 100 times with 15%—ethyl alcohol, after which energy treatment of the resulting dilution is carried out for 40 minutes by passing an electric current under the action of a potential difference applied electrodes placed in the solution, which is proportional in amplitude to the amplified combined total signal formed by adding the resulting signals—the differences in the bioelectric potentials of the human brain, detected by three healthy donors by electroencephalography. Then, the resulting dilution, treated with the transformed biopotentials of the human brain, is diluted by successively reducing the initial concentration to obtain a C200 dilution in combination with an external mechanical effect—shaking each dilution.

The study was conducted on male BALB/c mice aged 2-2.5 months. (Research Institute of General Pathology and Pathophysiology, Russia, Moscow). Preparation 1 was administered to 10 mice for 5 days intragastrically at a dose of 10 ml/kg/day. In the control group (Preparation 2), purified water was administered to 10 mice for 5 days intragastrically at a dose of 10 ml/kg/day.

The level of anxiety was assessed in the “elevated plus maze” installation. This test is basic for studying the tranquilizing (anxiolytic) action of compounds and is designed to study the behavior of rodents in conditions of variable stressogenicity (with free choice of comfortable conditions), allowing to evaluate: the level of anxiety of the animal (by preference of darkness/light, fear of heights, intensity and dynamics of behavior “Peeping”). Normally, animals prefer to spend most of their time in closed (dark) arms of the labyrinth. The anxiolytic effect of the drug is estimated by the increase in the number of visits to open (light) sleeves and the time spent in them, without increasing the total motor activity.

The “elevated plus-maze” installation (NPK Open Science, Russia) consists of 4 sleeves which are crosswise diverging from the central area at a right angle. Two opposite sleeves are closed from the sides and ends of the opaque walls; the other two do not have walls. The floor of all sleeves and the central area is opaque. The central platform has dimensions of 5 by 5 cm. The length of the labyrinth's sleeves is 30 cm, width is 5 cm, and the height of the walls is 15 cm. The labyrinth is elevated above the floor by a height of 25 (30) cm.

The mouse was placed in the center of the maze and within 5 minutes the latent time of the start of movement, the number of entries into open and closed sleeves and the time spent by animals in open and closed sleeves, as well as the number of defecations were recorded.

The declared medicinal product (Preparation 1) recorded anxiolytic activity, which was expressed in a statistically significant decrease in 1.3 times the time spent in the closed arms of the maze (Table 23).

TABLE 23
Indicators of the behavior of male mice in the test “elevated plus
maze” against the background of the course of Preparation 1.

Group

| | Preparation 2 | |
|---|---|---|
| Factor | (purified water) | Preparation 1 |
| LP start of movement, | 9.53 ± 2.54 | 6.40 ± 2.32 |
| sec | | |
| Time in OA, sec | 14.60 ± 9.29 | 80.50 ± 27.61 |
| Open-arm entry, sec | 0.80 ± 0.59 | 1.20 ± 0.51 |
| Time in the centre, sec | 36.77 ± 11.62 | 20.95 ± 13.78 |
| Time in CA, sec | 248.62 ± 20.22 | 183.30 ± 9.05* |
| Closed-arm entry, sec | 3.20 ± 0.71 | 2.70 ± 0.86 |
| Total number of visits, | 4.00 ± 0.95 | 3.90 ± 1.14 |
| ea | | |
| bolus, ea. | 0.60 ± 0.27 | 0.40 ± 0.16 |

Note:
data are presented as M ± SEM.
*p < 0.05 compared with control;
LP is the latent period, OA is an open-arm, and CA is a closed-arm.

Thus, in the experiment performed, it was shown that Preparation 1 exhibits an anxiolytic effect, reducing the animals' fear of open lit space in the “elevated plus-maze” test.

### EXAMPLE 20

The study determined the effectiveness of the claimed medicinal product (Preparation 1) to have a nootropic effect, activating the effect on the cognitive activity of old animals. Preparation 1 is an energetically processed release-active form of polyclonal antibodies to the brain-specific protein S-100 affinity purified.

Preparation 1 was obtained in the following way. The original substance of antibodies to protein S-100 (5 mg/ml) is diluted 100 times with 15% ethyl alcohol, followed by energy treatment of the resulting dilution for 1 hour by passing an electric current under the action of a potential difference applied to the electrodes placed in the solution, which is proportional in amplitude to the amplified combined total signal formed by adding the resulting signals—the differences in the bioelectric potentials of the human brain, recorded from five healthy donors by the method of elect Roencephalography. Then, the resulting dilution, treated with the transformed biopotentials of the human brain, is diluted by successively reducing the initial concentration to obtain a C200 dilution in combination with an external mechanical effect—shaking each dilution.

In the study, white Wistar male rats (498-718 g, 21 months old) were used. Preparation 1 was administered to rats for 12 days intragastrically at a dose of 5 ml/kg/day. In the control group (Preparation 2), purified water was intragastrically administered to the rats for 12 days at a dose of 5 ml/kg/day.

The ability of the claimed medicinal product (Preparation 1) to exert an activating effect on the cognitive activity of old rats was studied in the “delayed alternation in Skinner box” test.

The experiment included two consecutive stages.

First stage: 10 training sessions, in which the levers were fed to the animal, the pressing of which was accompanied by food reinforcement. Regardless of which of the levers (left or right) was chosen by the animal, the food pellet fell out of the feeder. Subsequently, the levers were fed randomly with an interval of 5 seconds, the pressing of which was also accompanied by food reinforcement. If the animal did not “respond” to any of the levers provided to it (more than 30 seconds), the levers moved without providing food. The light signal was applied at the beginning of the experimental session, as well as throughout the session, the duration of which was 30 minutes.

In the second stage of the study, animals were tested in 10 sessions with “delayed” alternation. Each session consisted of 36 consecutive attempts, separated by a time interval of 10 seconds.

The testing scheme was as follows:

The animal was given one of the levers (left or right), when pressed, food fell out of the feeder, then the lever moved and after 2.5 seconds the animal was again provided with both levers. The rat had to learn to press the lever opposite to the one presented earlier. If the animal did not respond to a single or double lever presentation for 20 seconds, the levers moved without further reinforcement with food.

The studied drugs were administered at the second stage 60 minutes before each test session. On weekends only the preparation was administered without further testing.

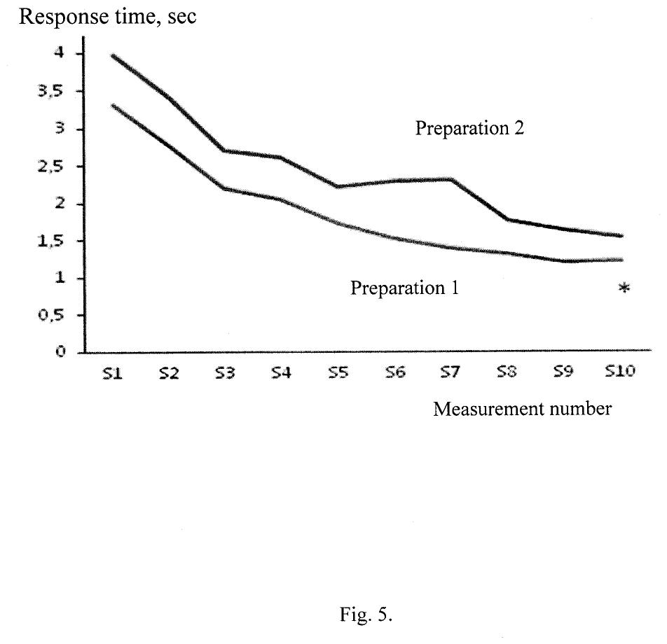

It was shown that in response to the administration of the Preparation 1, the response time of animals was significantly lower than that of the group of animals treated with purified water (control, Preparation 2) (FIG. 5).

Thus, it is shown that the claimed medicinal product (Preparation 1) has a stimulating effect on the cognitive activity of old animals, accelerating the process of their learning.

### EXAMPLE 21

The study determined the effectiveness of the claimed medicinal product (Preparation 1) to have a nootropic effect on the model of focal cerebral ischemia. Preparation 1 is an energetically processed release-active form of monoclonal antibodies to the brain-specific protein S-100 affinity purified.

Preparation 1 was obtained in the following way. The original substance of antibodies to protein S-100 (6 mg/ml) is diluted 200 times with 15% ethanol, followed by energy treatment of the resulting dilution for 1 hour by passing an electric current under the action of a potential difference applied to the electrodes placed in the solution, which is proportional in amplitude to the amplified combined total signal formed by adding the resulting signals—the differences in the bioelectric potentials of the human brain, recorded from two healthy donors by the electroencephalography. Then, the resulting dilution, treated with the transformed biopotentials of the human brain, is diluted by successively reducing the initial concentration to obtain a C100 dilution in combination with an external mechanical effect—shaking each dilution.

The study used rats male line Sprague-Dawley rats weighing 277-339 g (Porsolt SAS, Le Genest-Saint-Il, France). Preparation 1 was administered to 24 rats for 49 days intragastrically at a dose of 10 ml/kg/day. In the control group (Preparation 2), 24 rats were intragastrically injected for 49 days with purified water at a dose of 10 ml/kg/day. Drug administration was started 1 hour after the pathology simulation and then daily, not earlier than 1 hour before the behavioral tests.

Focal cerebral ischemia was reproduced according to the method described by Longa E. Z. et al. [Longa E Z, Weinstein P R, Carlson S, Cummins R. Reversible middle cerebral artery occlusion without craniectomy in rats. Stroke. United States; 1989 January; 20(1):84-91] as modified by Esneault et E. et al. [Esneault E, Castagne V, Moser P, Bonny C, Bernaudin M. D-JNKi, a peptide inhibitor of c-Jun N-terminal kinase, promotes functional recovery after transient focal cerebral ischemia in rats. Neuroscience. United States; 2008 March; 152(2):308-20].

In the dynamics of the study evaluated the following indicators of animals:
neurological status/deficiency—a survey on a scale of Bederson et al. (1986): 6 measurements (1, 3, 7, 14, 21, 28 days after surgery):

The test included 14 measurements: at the beginning, spontaneous walking and rotation of animals on the paretic side were evaluated. Next, the rat (holding the tail) was placed on a rough surface and gently pushed to the homolateral and contralateral sides to assess the resistance of the animal to the jolt provided. At the end, the rat was hung by the tail (successively, using the experimenter's right and left hands) and raised above the bench to assess the rotation of the animal's body, as well as the flexion of the fore and hind limbs.

Each measurement was rated on a scale of 0 to 2 (0=no response or completely deviating response, 1=weak or deviating response, 2=normal response). The maximum neurological status of animals, characterized by the absence of injuries/lesions on the part of the neurologist of the system was equal to (was) 28 points.
Histological studies—on the 3rd and 28th day after surgery:

The brain of animals was frozen in a solution of isopentane (Sigma) at −20° C. Then the samples were placed in Tissue-Tek® O.C.T. (OCT125, CML, France) and prepared coronary sections (20 μm), which were then stained with thionin (T7029, Sigma, 0.05%) for 5 minutes. Lesion volumes were assessed by scanning histological histological sections using ImageJ software (http://rsb.info.nih.gov/ij/). The volume of the lesion was normalized relative to the volume of the whole brain and edema (the difference between the volume of the homo(ipsi)-contralateral hemispheres).

One of the secondary performance criteria was an indicator of the mass of animals, which was assessed daily: one day before the pathology simulation and then for 37 days.

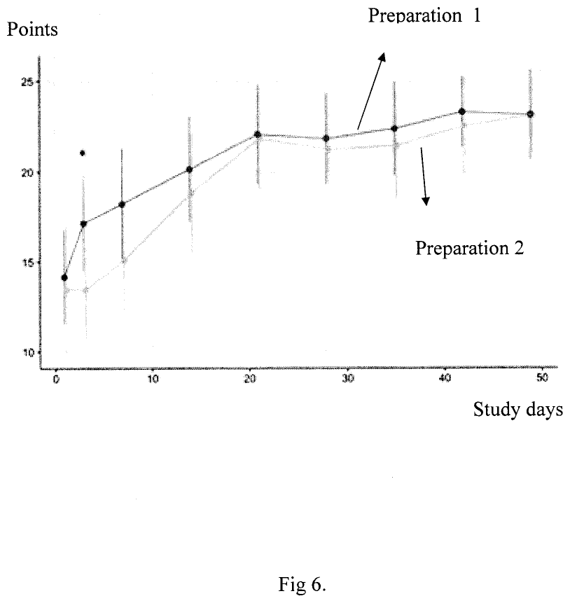

According to the neurological scale, the group of animals receiving the claimed medicament (Preparation 1) recovered faster than the rats receiving the control sample: statistically significant intergroup differences were found already on the third day after the operation (FIG. 6).

The results of histological studies revealed partial preservation of the basal (caudate) ganglia in 2 out of 3 rats treated with Preparation 1, 28 days after the stroke. For one animal, a decrease was noted compared with control of the anterior and posterior distribution of the cortical lesion adjacent to the hippocampus. Intergroup differences in terms of the “mass of animals” indicator were not detected.

Thus, the study showed the nootropic activity of the claimed medicament (Preparation 1), which consists in accelerating the recovery of neurological status and reducing the severity of histological indicators of neurodegenerative processes in the brain of animals with experimental focal cerebral ischemia.

### EXAMPLE 22

The study determined the effectiveness of the claimed medicinal product (Preparation 1) on a model of hemorrhagic stroke. Preparation 1 is an energetically processed release-active form of polyclonal antibodies to the brain-specific protein S-100 affinity purified.

Preparation 1 was obtained in the following way. The original substance of antibodies to protein S-100 (3.5 mg/ml) is diluted 200 times with 15% ethanol, followed by energy treatment of the resulting dilution for 1 hour by passing an electric current under the action of a potential difference applied to the electrodes placed in the solution, which is proportional in amplitude to the amplified combined total signal formed by adding the resulting signals—the differences in the bioelectric potentials of the human brain, recorded from five healthy donors by the method of electroencephalography.

In the study, male Wistar rats (180-200 g) were used. Preparation 1 was administered to 20 rats for 14 days intragastrically at a dose of 5 ml/kg/day. In the control group (Preparation 2), purified water was administered intragastrically to 20 rats at a dose of 5 ml/kg/day for 14 days. Drug administration was started 1 hour after the pathology simulation and then daily, not earlier than 1 hour before the behavioral tests. The comparison drug nimodipine (Preparation 3) was also administered to 20 rats for 14 days intragastrically at a dose of 0.1 ml/kg/day. In addition, the study had a negative control group (Preparation 4)—rats with experimental pathology (n=20), which were intra-gastric injection of physiological saline (5 ml/kg/day) for 14 days.

Hemorrhagic stroke (intracerebral hemorrhage) in the left caudate nucleus of the brain in rats was modeled 4-4.5 hours before the administration of the preparations according to a modified method of W. Deinsberger et al. [Deinsberger W., Vogel J., Kuschinsky W. et al. Experimental intracerebral hemorrhage: description of a double injection model in rats//Neurol. Res. 1996.—No. 5]. This method involves the double introduction of autologous blood into the specified structure of the brain of animals. The hemorrhagic stroke simulated in this way in rats in localization corresponds to intracerebral hemorrhages that occur in humans when the lenticularity arteries rupture. The total volume of hematoma in animals corresponds to approximately 40-50 ml³ in terms of human body weight.

In the dynamics of the study evaluated the following indicators of animals:
life expectancy and survival—observation for 14 days after surgery.
approximately exploratory behavior—open field test: on the 1st, 3rd, 7th and 14th day after surgery.
neurological status/deficiency—Menzies (1992) examination, angular rotation test, asymmetric use of the forelimbs: on the 1st, 3rd, 7th and 14th day after surgery.
cognitive functions—the test “conditioned passive avoidance reflex” (CPAR) after assessing the neurological status: on the 1st, 3rd, 7th and 14th day after the operation.
histological studies were carried out for the groups of animals “negative test (Preparation 4)”, “Preparation 1” and “Nimodipine (Preparation 3)”-3 rats from each group on the 3rd and 7th day after the operation.

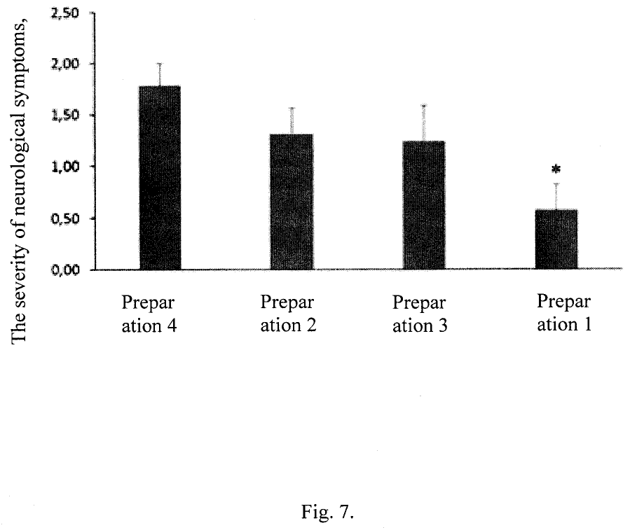

The course intragastric administration to the rats of the claimed medicinal product (Preparation 1) was accompanied by a positive dynamic of neurological symptoms in the post-stroke period. The mean score on the neurological scale in these animals was minimal by the end of observations and was statistically significantly different from that in individuals from the negative control group (Preparation 4) and in rats treated with purified water (Preparation 2) (FIG. 7). The use of Preparation 1 led to the normalization of indicators of motor preference for animals with intracerebral hemorrhage, which was manifested in a decrease in the percentage of turns to the left side while simultaneously increasing the proportion of right-hand turns. The improvement in motor preference indices of rats of this group was expressed in a slight increase in the frequency of friendly use of the forelimbs or the right forepaw. As a result of pairwise comparison of groups, the following statistically significant differences in motor preference indices were established: “Preparation 1”—compared to “negative test (Preparation 4)” (by 25.61%, p<0.001); “Nimodipin (Preparation 3)”—compared to “negative test (Preparation 4)” (by 24.22%, p<0.001).

A positive effect on the cognitive functions of animals was revealed on the 7th day of the study: the total time spent in the dark compartment of the camera of the passive avoidance reaction was [s, M (Q1; Q3)]: the group “negative test (Preparation 4)”—157.00 (101.75; 171.75), the group “purified water (Preparation 2)”—155.00 (0.00; 173.00), the group “Nimodipine (Preparation 3)”—171.00 (163.00; 175.00), the group “Preparation 1”—142.00 (0.00; 171.00).

The area of the hemorrhagic lesion 3 days after the modeling of intracerebral hemorrhage was greatest in rats with saline (“negative test”, 4.3±1.3 μm2) compared with this parameter in individuals treated with the Nimodipine reference drug, by 30, 2%, compared with animals treated with Preparation 1—by 48.8%.

By the 7th day after stroke modeling, animals of all groups showed a general tendency to decrease the area of the hemorrhagic lesion area (compared with the 3rd day of the study): “negative control (Preparation 4)”—by 62.8%, “Nimodipine (Preparation 3)”—by 16.7%, “Preparation 1”—by 80.8%.

The effects of the drug on the life expectancy, survival, and orienting-exploratory behavior of animals in this study were not identified.

Thus, the effectiveness of the experimental hemorrhagic stroke, which is expressed primarily in the positive dynamics of neurological symptoms in the post-stroke period and the normalization of motor preference indices for intracerebral hemorrhage, was shown for the claimed medicament (Preparation 1).

### EXAMPLE 23

The study determined the effectiveness of the claimed medicinal product (Preparation 1) on a model of photo-induced thrombosis. Preparation 1 is an energetically processed release-active form of polyclonal antibodies to the brain-specific protein S-100 affinity purified.

Preparation 1 is the released-active form of polyclonal antibodies to S100 protein (S100 pAb), obtained by mixing three different serial dilutions of an S100 pAb stock (matrix solution) prepared in an aqueous solvent (concentration of 3.0 mg/mL) The following dilutions were mixed at a 1:2:1 (v/v) ratio:

1) the stock (matrix) solution diluted by 100¹⁵ times (or to C15). 2) the stock (matrix) solution diluted by 100³⁰ times (or to C30), each time in combination with external mechanical treatment (shaking).

3) the stock (matrix) solution diluted by 100⁵⁰ times (or to C50), each time in combination with external mechanical treatment (shaking).The above mixture was energy-treated by 20-minute exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 6 healthy donors by the electroencephalography.

Then, the resulting dilution, treated with the transformed biopotentials of the human brain, is diluted by successively reducing the initial concentration to obtain a C100 dilution in combination with an external mechanical effect—shaking each dilution.

White Wistar male rats (190-210 g) for 5 days before pathology simulation (last time 1 hour before thrombosis) and for the next 9 days of the postischemic period intragastrically injected purified water at a dose of 5 ml/kg/day ((Preparation 2, control, n=12) or Preparation 1 at a dose of 5 ml/kg/day (n=12). The last introduction was carried out 40 minutes before testing. Additional controls in the study were the group of sham operated (Preparation 3, n=12) and intact (Preparation 4, n=12) animals.

Local photochemical damage to the prefrontal cortex in rats was created according to a protocol developed by Watson et al. in the modification of I.V. Viktorov, which allows to compare the morphological, neurochemical and functional disorders occurring in the central nervous system, and to adequately evaluate the effect of pharmacological agents that stimulates regenerative processes. A feature of the model is the presence in experimental animals of memory impairment and ability to learn without disorders of motor coordination and muscle tone.

Before creating an ischemic focus in the brain of rats, each experimental animal developed a “conditioned passive avoidance reflex (passive avoidance reaction)” for three days in an experimental chamber consisting of two compartments: the larger, illuminated (with transparent walls) 30×30×25 cm in size, and the smaller, darkened, 15×15×18 cm in size with an electrically conductive floor, to which electrical pain stimulation with a force of 1.0 mA was applied (pulse duration—1 s, interval between pulses—2 s).

On the 4th day of the post-ischemic period from each group of animals, by random sampling, 2-3 hours after the last injection of drugs, the brain was collected from 2 rats to estimate the area of the lesion. On the 5th and 9th day, 1 hour after administration of Preparation 1 or purified water (Preparation 2), the remaining animals were tested in passive avoidance reaction. On the 9th day, after checking the degree of preservation of passive avoidance reaction, for 1 hour under chloral hydrate anesthesia, rats were removed from the experiment for subsequent neurohistological studies.

It is shown that the claimed medicinal product (Preparation 1) ensured the preservation of the memory trace in animals with ischemic brain damage at a level equal to that of intact and sham-operated animals (Table 24).

TABLE 24
The latent period of entry of rats into a dark chamber in the test
“passive avoidance reaction” on the model of photo-induced
thrombosis against the background of the course of Preparation 1.

| | | 5th day after | 9th day after |
|---|---|---|---|
| | | photo- | photo- |
| Factor | basic data | thrombosis | thrombosis |
| Photothrombosis + | 127.5 ± 29.87 | 132.43 ± 22.86 | 137.62 ± 13.48 |
| Preparation 1 | | | |
| Photothrombosis + | 114.71 ± 18.30 | 43.85 ± 2.71* | 49.54 ± 11.16* |
| Preparation 2 | | | |
| (purified water) | | | |
| Preparation 3 (sham | 136.93 ± 23.45 | 129.60 ± 20.19 | 128.00 ± 15.23 |
| operated rats) | | | |
| Preparation 4 | 126.70 ± 21.71 | 145.22 ± 22.61 | 137.00 ± 16.30 |
| (intact rats) | | | |

Note:
data are presented as M ± SEM;
*p < 0.05 compared with baseline (two-way ANOVA with Tukey's post-hock criterion).

The size of the stroke focus in animals treated with the MAP preparation was 1.935±0.144 mm3 vs 3.439±0.248 mm3 in the control. At the same time, in the group treated with Preparation 1, pathological changes in the brain (tissue destructive and necrobiotic cells) were less pronounced than in animals of the control group (Preparation 2). The brain in sham operated (Preparation 3) and intact animals (Preparation 4) had no pathological changes.

Thus, it is shown that the claimed medicinal product (Preparation 1) has a pronounced neuroprotective effect: it ensures the preservation of the memory trace in the postyschemic period, and also reduces the amount of damage to the focal focus of the brain and prevents the occurrence of pathological changes.

### EXAMPLE 24

The study determined the effectiveness of the claimed medicinal product (Preparation 1) to show a sedative effect on the model of development and extinction of long-term post-tetanic potentiation. Preparation 1 is an energetically processed release-active form of polyclonal antibodies to the brain-specific protein S-100 affinity-purified.

Preparation 1 is the released-active form of polyclonal antibodies to S100 protein (S100 pAb), obtained by mixing three different serial dilutions of an S100 pAb stock (matrix solution) prepared in an aqueous solvent (concentration of 3.5 mg/mL) The following dilutions were mixed at a 2:1:1 (v/v) ratio:

1) the stock (matrix) solution diluted by 100³⁰ times (or to C30).

2) the stock (matrix) solution diluted by 100⁵⁰ times (or to C50), each time in combination with external mechanical treatment (shaking).

3) the stock (matrix) solution diluted by 100¹⁰⁰ times (or to C100), each time in combination with external mechanical treatment (shaking). The above mixture was energy-treated by 2-hours exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 3 healthy donors by the electroencephalography.

The experiments were carried out on 20 white Wistar male rats (150-200 g). After decapitation, the rat brain was placed in a pre-aerated carbogen (95% O₂/5% CO₂, cooled to 4° C.), the carbogen was fed into the solution directly from a balloon under pressure, through an aeration stone) artificial cerebrospinal fluid (ACSF): 126 mM NaCl, KCl 3.50 mM, NaH₂PO₄ 1.25 mM, NaHCO₃ 25 mM, CaCl2 2.00 mM, MgCl₂ 1.30 mM, glucose 10 mM, pH 7.4. Using the NVSL Vibroslicer vibrotome, parasagittal (L=2.5-3 5 mm) hippocampal sections with a thickness of 350 μm were made.

After manufacturing, the sections were placed in an incubation chamber with bilateral superperfusion (10 ml/min) with aerated ACSF at room temperature before the start of the experimental procedures for at least 2 hours. The sections obtained from each animal with numbers 1-10 were distributed between groups 3 and 4, the sections obtained from each animal with numbers 11-20 between groups 1 and 2.

Experimental Groups:

Group 1—Preparation 1, perfusion during the entire registration period, without pre-incubation (n=10);

Group 2—Preparation 1, perfusion during the entire registration period, pre-incubation with β-amyloid peptide (n=10).

Group 3—purified water, perfusion during the entire period of registration, without preincubation (n=10);

Group 4—purified water, perfusion during the entire period of registration, preincubation with β-amyloid peptide (n=10);

Activity recording in the incubation chamber at a temperature of 31-33° C. was made using microelectrodes from borosilicate glass filled with ACSF. To record the field activity of pyramidal neurons of the CA1 field of the hippocampus, electrodes with a resistance of 1.2-1.5 MOhm were used, a tungsten stimulating bipolar electrode was placed in the area of Schaffer collaterals. Using electrical stimulation, local field potentials were induced.

For induction of the population spike, single stimuli of 150-200 μA were used. For induction of only a synaptic response, the stimulation amplitude (30-100 μA) was chosen so that the excitatory postsynaptic potential (APSP) was 1/3 of the maximum. Long-term posttetanic potentiation was caused by electrical stimulation with two series of stimuli with a frequency of 100 Hz for a duration of 1 s with an interval of 60 s. To assess the effectiveness of long-term synaptic potentiation, the rate of change (slope) of the front edge of the EPSP was measured.

After registration of the base level of neural responses and adjustment of the stimulation parameters, experimental preparations were added to the incubation medium washing the slice. The control (baseline) activity of the slice was recorded for 5 minutes before and 15 minutes after the preparation was administered on Wednesday. After registration of the base activity, the delivery of irritating stimuli and the production of DTPA began.

To assess the effect of drugs on the toxic effect of beta amyloid peptide 1-42 (Aβ1-42), sections of experimental groups 2 and 4 were pre-incubated for one hour in medium containing 1 μM peptide.

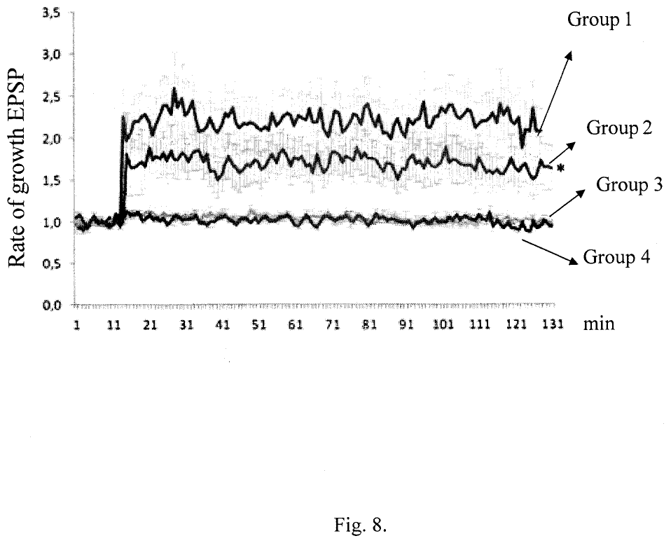

Induction of DTPA during incubation of the slice in the medium with Preparation 1 led to an increase in the growth rate of EPSP by 76±28% in the first 20 minutes after stimulation. DTPP persisted for 2 hours with a slight (unreliable) drop to 62±24% (FIG. 8). At the same time, incubation in the medium containing purified water in the first 20 min reached values of 109±32% and remained at this level for the next 2 hours.

None of the studied drugs had an effect on the toxic effect of Aβ1-42.

Thus, the claimed medicament (Preparation 1) has an inhibitory effect on the synaptic plasticity of the neurons of rat hippocampus undergoing sections, which are consistent with the previously identified sedative effect of the drug.

### EXAMPLE 25

The study determined the effectiveness of the claimed medicinal product (Preparation 1) in the treatment of cerebral palsy, on the model of lesions of rat pups with ibotenic acid.

Preparation 1 was containing a 1:1 v/v energy-treated mixture of the following:

A) the released-active form of polyclonal antibodies to S100 protein (S100 pAb), obtained by is diluted 200 times with 15% ethanol (concentration of 5 mg/mL).
B) the released-active form of polyclonal antibodies to cannabinoid receptor type 1 (CB1 pAb), obtained by is diluted 200 times with 15% ethanol (concentration of 4 mg/mL) The above mixture was energy-treated by 3-hours exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 5 healthy donors by the electroencephalography.

The study was conducted on 74 Wistar rats of both sexes (n=74; 43 in, 31f). To obtain a model of cerebral palsy, rat pups of the 5th postnatal day (the day of birth is considered to be PND zero) were stereotactic IBA. During the operations, the rat pups were anaesthetized by the combined administration of a ditor (Orion Pharma, Finland) at a dose of 100 mg/kg and Zoletil 50 (Virbac Sante Animale, France) at a dose of 5 mg/kg. The scalp of the rats was incised and 1 μl of the solution was injected bilaterally (2.5 μg IBA in 1 μl of saline solution or only 1 μl of saline solution) between the non-accreting frontal and pariental bones of the skull. The axon tracts of the corpus callosum (corpus callosum) in the area between the sensorimotor cortex (front paws) and the striatum were the targets for damage. At the end of the stereotactic operation and suturing, the animal was administered anti-sedan (Orion Pharma, Finland) at a dose of 2.5 mg/kg to more quickly get out of anesthesia and minimize side effects.

The survival rate of rats was □95%. Upon achievement of the experimental animals of the 28th IPA, the following groups were formed to conduct the research:

1. Intact control. A group of rats of both sexes without any pharmacological or surgical intervention, which was in conditions similar to the experience (n=16, 10m and 6f);

2. Control group 1 (sham operated rats). A group of rats of both sexes, which on the 5th postnatal day underwent stereotactic surgery with the introduction of saline into the WBW and the subsequent, starting from the 28th postnatal day, oral administration of saline (n=10, 6m and 4f);

3. Control group 2 (hereinafter referred to as “IBA”). A group of rats of both sexes, which on the 5th postnatal day underwent stereotactic surgery with the introduction of IBA in PBS and subsequent, starting from the 28th postnatal day, oral administration of saline in a volume of 0.3 ml/animal course for 5 days (n=9, 5m and 4f);

4. The experimental group (the “Preparation”). A group of rats of both sexes, which on the 5th postnatal day underwent stereotactic surgery with the introduction of IBA in PBS and subsequent, starting from the 28th postnatal day, oral administration of Preparation 1 in a volume of 0.3 ml/animal course for 5 days (n=12 7m and 5f);

The results of the study:

Body Weight and Clinical Examination

With daily monitoring, a fairly harmonious and correct development of rats in all groups was established. Opening the ears and eyes, teething and the appearance of coat was within standard time limits. During the clinical examination, the determination of the habit, assessment of the skin and visible mucous membranes was carried out. In all groups, the condition of the animals at the time of inspection was satisfactory.

Behavior Testing:

At the end of the injection of the test samples, behavioral studies of experimental animals were carried out for 4 weeks. To assess the degree of neurological disorders obtained, the following behavioral tests were performed: “open field”, “tapering lane”, and nutritional test.

Nutritional Test

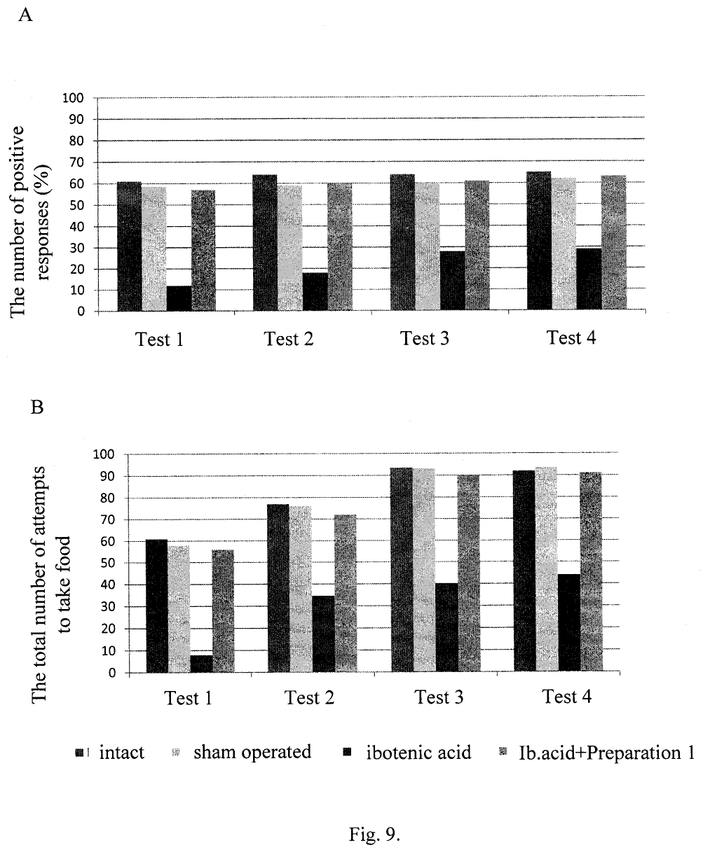

A study of the behavior of experimental animals in the food procurement test showed that intact animals performed the test with a high percentage of positive reactions, which remained unchanged throughout all four tests (FIG. 9A). Falsely operated animals and animals of the experimental group (Preparation) showed in the first test a smaller percentage of positive reactions than intact ones, and the percentage of positive reactions in the rats of control group 2 (IBA) was extremely small (FIG. 9). In subsequent testing, an increase in the percentage of correct reactions was observed.

Damage to PVBV and bothenic acid led to a marked deterioration in the performance of experimental food-producing test by experimental animals. However, it should be noted that in the experimental group (Preparation 1) the percentage of positive reactions already in the first test was comparable to the indicator of the group of falsely operated animals and with further testing remained at a high level.

Test “Tapering Track”

Testing of experimental animals on the installation “DM” was performed twice, at 33 (Test 1) and 55 (Test 2) PND.

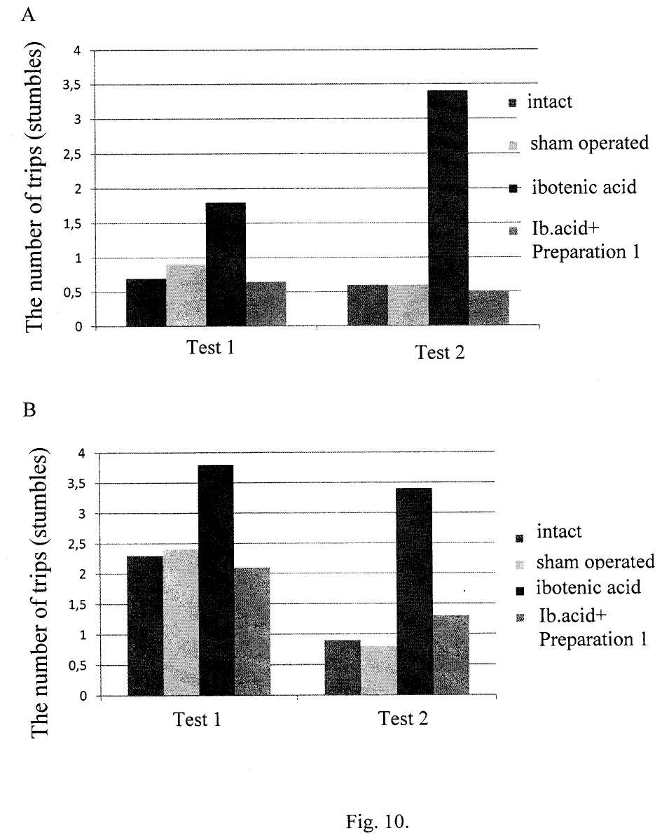

The number of front and hind limb margins in the IBA group was the highest among all groups in both tests. The number of purchases in animals of the experimental group (Preparation) was significantly different from the group “IBA” (FIG. 10).

Open Field Test

Testing of motor activity in the “OP” was conducted during the study twice, immediately after the introduction of the samples of the tested drugs and after the last PDT.

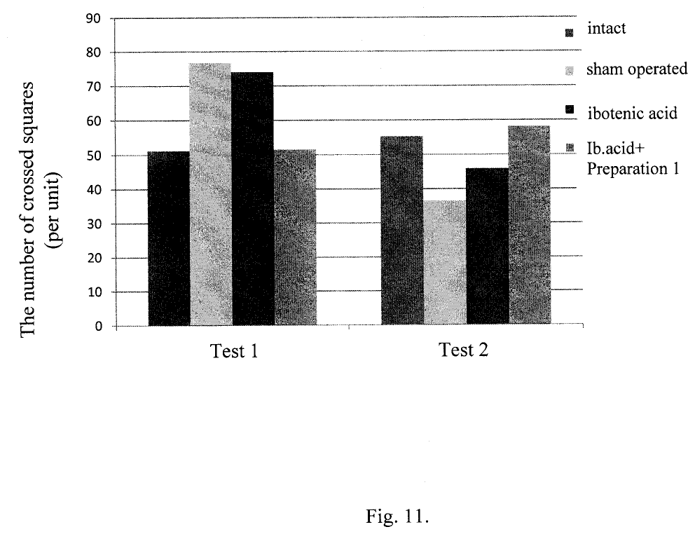

The change in the amount of motor activity was detected when comparing between tests within each group. When calculating the number of crossed squares, it was shown that significant differences were between the falsely operated group and the experimental group (Preparation 1) (FIG. 11).

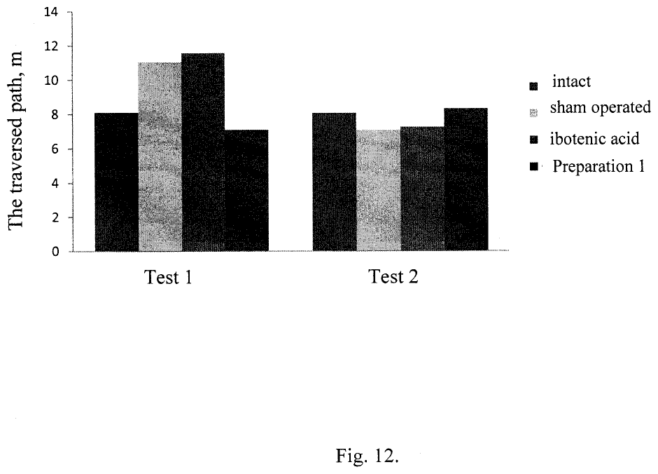

The recording and analysis of the motor activity of experimental rats using the Any-maze program showed that the change in the distance covered (FIG. 12) had the same character as the change in the number of crossed squares, although the movement of the animal inside the squares was taken into account when measuring the distance traveled (treading, rotations, etc.).

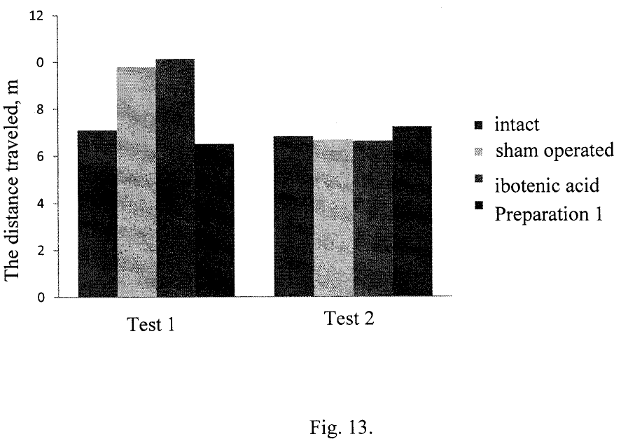

A similar pattern of changes was observed when analyzing movements in the central zone of the “open field”. There were no significant differences in the distance covered in the central zone when comparing between groups (FIG. 13).

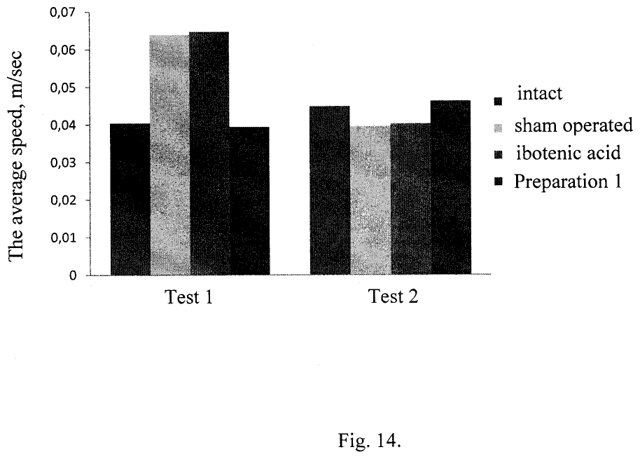

Another analyzed parameter was the average speed of movement in the installation (FIG. 14).

Conclusion:

Oral administration of the claimed medicament restored disorders caused by intracerebral administration of ibotenic acid in the nutritional test, there was a positive dynamics of changes in motor activity in the open field test, and the drug showed an improvement in the emotional state of the rat in the open field test, with repeated testing of the tapering track number hind limbs did not differ from the model group. The effect of the introduction of the drug was the most significant.

### EXAMPLE 26

The study determined the efficacy and safety of the claimed medicinal product (Preparation) in helping children with speech disorders. The study was conducted on the basis of a specialized school for children with speech development problems.

Preparation 1 was containing a 1:1 v/v energy-treated mixture of the following:

A) the released-active form of polyclonal antibodies to S100 protein (S100 pAb), obtained by is diluted 100 times with 15% ethanol (concentration of 8 mg/mL)

B) the released-active form of polyclonal antibodies to cannabinoid receptor type 1 (CB1 pAb), obtained by is diluted 200 times with 15% ethanol (concentration of 6 mg/mL) The above mixture was energy-treated by 1-hours exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 3 healthy donors by the electroencephalography.

Then, the resulting dilution, treated with the transformed biopotentials of the human brain, is diluted by successively reducing the initial concentration to obtain a C200 dilution in combination with an external mechanical effect—shaking each dilution.

Material and methods of examination. The study involved 10 children of both sexes (6 boys and 4 girls) aged 7-8 years (younger students in grades 1-2) who have speech disorders classified by ICD-10 as Specific disorders of speech and language development (F80), including: Specific disorder of speech articulation (F80.0); Expressive speech disorder (F80.1); Receptive speech disorder (F80.2); Other disorders of speech and language development (F80.8). All children were prescribed a course of the stated drug (Preparation 1), 1 tablet 3 times a day for 12 weeks.

When children were included in the study, testing was conducted to diagnose speech disorders according to the method of T. A.

Fotekova, T. V. Akhutina, 2002. Speech therapy includes a series of tests that determine the level of formation of different sides of speech, and consists of two blocks.

Block 1—aimed at evaluating expressive speech, consists of 5 series, allowing to assess:

1. Sensomotor speech level: Phonemic perception; Articulatory motor skills; Sound subscription; Formation of the sound-syllable structure of the word.

2. Active dictionary.

3. Word formation skills.

4. The grammatical structure of speech.

5. States of connected speech.

Block 2 is aimed at assessing impressive speech and consists of 2 series:

1. Understanding the meaning of words.

2. Understanding of logical and grammatical structures.

The study included children, whose average score in all areas was from 50% to 79.9%, that is, it corresponds to III (79.9-65%) or II (64.9-50%) to the success rate of the method.

Efficacy Endpoint:

Primary endpoint: Performing speech examination methods in groups after 12 weeks of treatment compared with the initial state; The average percentage of success in performing speech tests in groups after 12 weeks of treatment compared with baseline.

Secondary endpoint: The proportion of children with an increase in the level of success (Increase in the level of success (depending on the percentage expression of quality)—transition from level II (64.9-50%) to level III (79.9-65%) or from level III to level IV (100-80%);

Safety criteria: The presence and nature of adverse events during the period of therapy, their connection with the drug intake.

Prohibited adjunctive services: The following drugs are not allowed 1 month prior to being included in the study and during the course of the study: Psychoanaleptics (ATX group—N06), including psychostimulants and nootropic drugs.

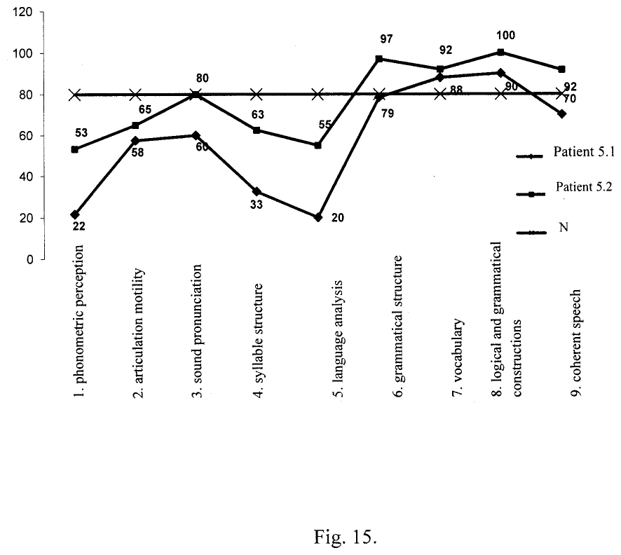

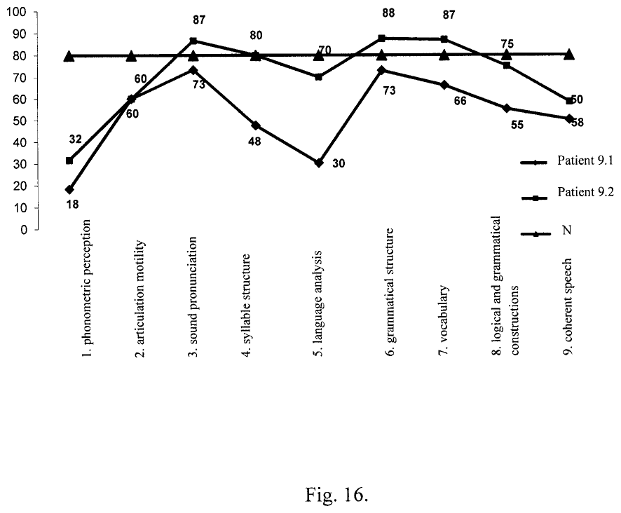

The results of the study: The results of the study of the effectiveness of the drug in relation to various options for violation of speech formation in children are shown in FIGS. 15 and 16.

FIG. 15 shows the dynamics of speech indicators in a child of 7 years and 8 months, mostly related to violations of speech perception, articulation motility and sound pronunciation, while vocabulary, grammatical structure and grammatical structures reached the norm. As a result of the therapy, the articulation and pronunciation of sounds almost reached the norm, while the indicators of the formation of the sound-syllable structure of the word also significantly improved. In general, the dynamics of the success rate reached high reliability in almost all indicators of the survey methodology.

FIG. 16 shows the clinical example of a patient aged 8.5 years with an initial primary deficit in terms of understanding the syllable structure of a word, language analysis, which caused the grammatical structure of the spoken language, the amount of vocabulary. After 12 weeks of application of the claimed Drug, the situation has positively changed and the marked irregularities in the formation of expressive speech indicators (syllable structure of the word, language analysis) almost reached the norm, which affected the quality of active speech.

Tables 25 and 26 show the dynamics of the percentage of success and the change in the level of success in terms of speech studies in children of the entire surveyed group (N=10).

TABLE 25
Dynamics of the percentage of success in children
in terms of speech studies.
The average percentage of success of the implementation
of speech survey methods

| patient | visit 1 | visit 2 |
|---|---|---|
| SS_120901 | 47.2% | 63.3% |
| SS_120902 | 76.7% | 89.9% |
| SS_120903 | 62.1% | 82.1% |
| SS_120904 | 47.6% | 63.7% |
| SS_120905 | 73.4% | 89.6% |
| SS_120906 | 61.1% | 78.2% |
| SS_120907 | 46.6% | 61.6% |
| SS_120908 | 70.3% | 86.8% |
| SS_120909 | 62.6% | 77.8% |
| SS_120910 | 79.7% | 91.2% |

Analysis of variance for repeated measurements
Type 3 Tests of Fixed Effects

| Effect | Num DF | Den DF | F Value | Pr > F |
|---|---|---|---|---|
| Visit | 1 | 9 | 469.54 | <.0001 |

descriptive statistics

| visit | N Obs | average | Std. dev. |
|---|---|---|---|
| visit 1 | 10 | 62.74% | 12.42 |
| visit 2 | 10 | 78.41% | 11.71 |

TABLE 26
Dynamics of the level of success in children in terms of speech research.
Change in the level of success of the speech survey method

| patient | Success rate, visit 1 | Success rate, visit 2 |
|---|---|---|
| SS_120901 | I | II |
| SS_120902 | III | IV |
| SS_120903 | II | IV |
| SS_120904 | I | II |
| SS_120905 | III | IV |
| SS_120906 | II | III |
| SS_120907 | I | II |
| SS_120908 | III | IV |
| SS_120909 | II | III |
| SS_120910 | III | IV |

Conclusion. The use of the claimed medicinal product in children with different types of speech development disorders contributes to the improvement of articulatory motility and sound pronunciation, as well as demonstrates effectiveness in terms of correct formation of the grammatical structure of the spoken connected speech, vocabulary volume.

After a course of treatment with the preparation according to the present invention, already after 12 weeks, objectively using speech therapy techniques with high certainty, an increase in the percentage and level of success in the formation of speech was observed, which had a positive effect on children's learning and psychological adaptation in the children's team. There were no undesirable side effects of the drug, it was well tolerated throughout the treatment.

### EXAMPLE 27

The study determined the efficacy and safety of the claimed medicinal product (Preparation) in the treatment of patients with infantile cerebral paralysis (ICP).

Preparation 1 was containing a 2:1 v/v energy-treated mixture of the following:

A) the released-active form of polyclonal antibodies to S100 protein (S100 pAb), obtained by mixing three different serial dilutions of an S100 pAb stock (matrix solution) diluted in an aqueous solvent (concentration of 2.5 mg/mL) The following dilutions were mixed at a 1:1:1 (v/v) ratio:

1) the stock (matrix) solution diluted 100¹² by (or to C12).

2) the stock (matrix) solution diluted by 100³⁰ times (or to C30), each time in combination with external mechanical treatment (shaking).

3) the stock (matrix) solution diluted by 100⁵⁰ times (or to C50), each time in combination with external mechanical treatment (shaking).

B) the released-active form of polyclonal antibodies to cannabinoid receptor type 1 (CB1 pAb), obtained by is diluted 200 times with 15% ethanol (concentration of 4.5 mg/mL) The above mixture was energy-treated by 3-hours exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 2 healthy donors by the electroencephalography.

Material and methods of examination. The study involved 7 children (average age of patients was 3.8±0.46 years, boys 6 people, girls 1 person) suffering from various forms of cerebral palsy: spastic diplegia, hyperkinetic form, atonic-astatic form, hemiparetic form. In some patients, mental and speech development was delayed.

Patients were prescribed the claimed medicament according to the scheme 10 cap*6 times a day for 1 week, then 10 cap*3 times a day for 2-8 weeks every day. Total duration of the study was 2 months (8 weeks).

The dynamics of motor dysfunctions was assessed using the GMFM-66 Global Motor Function Measurement Scale (Gross Motor Function Measure).

TABLE 27
Dynamics of indicators on the GMFM-66 scale before and after therapy.

| No of | GMFM | GMFM | GMFM | |
|---|---|---|---|---|
| the | before | after 1 | after 2 | |
| patient | therapy | month | month | Subjective |
| 001 | 25 | 29 | 32 | Improved muscle tone is better sitting, has become |
| | | | | more calm, adapted to the environment |
| 002 | 117 | 141 | 152 | Improved muscle tone, improved range of motion in |
| | | | | some joints, ataxia decreased, fine motor skills |
| | | | | improved, understanding of reversed speech |
| | | | | improved, vocabulary increased, switchability |
| | | | | increased |
| 003 | 133 | 137 | 138 | Physically stronger, decreased pseudobulbar |
| | | | | syndrome, improved adaptation to the environment, |
| | | | | became less irritable |
| 004 | 130 | 138 | 146 | Improved muscle tone, increased range of motion in |
| | | | | some joints, improved concentration, adaptation to |
| | | | | the environment. Increased vocabulary |
| 005 | 50 | 60 | 60 | Improved muscle tone, decreased joint stiffness, |
| | | | | decreased ataxia and propulsions when walking. He |
| | | | | began to walk more confidently and over long |
| | | | | distances. |
| 006 | 125 | 131 | 132 | Well adapted to the environment, improved muscle |
| | | | | tone, slightly decreased dysphonia |
| 007 | 146 | 152 | 165 | Improved muscle tone, increased range of motion in |
| | | | | some joints, improved understanding of reversed |
| | | | | speech, increased vocabulary. |

Conclusions. Patients received the study drug on the background of a wide range of rehabilitation measures, including physiotherapy techniques, classes with speech therapist, exercise therapy, massage.

In general, there was a predominantly positive effect on muscle tone with an increase in the range of motion of the joints, in two patients a decrease in the severity of pseudobulbar symptoms was noted. Also, the majority of patients improved their adaptation to the environment, the improvement of speech production, and their understanding of reversed speech.

There is a delayed effect of the drug: in those patients who continued taking the drug after discharge, there was a longer retention effect achieved during inpatient treatment compared with previous observations of patients before the appointment the claimed medicament.

### EXAMPLE 28

The study determined the efficacy and safety of the claimed medicament (Preparation) in the treatment of attention deficit hyperactivity disorder (ADHD) in children during a double-blind, placebo-controlled study.

Preparation 1 was containing a 1:1 v/v energy-treated mixture of the following:

A) the released-active form of polyclonal antibodies to S100 protein (S100 pAb), obtained by is diluted 200 times with 15% ethanol (concentration of 7 mg/mL)

B) the released-active form of polyclonal antibodies to cannabinoid receptor type 1 (CB1 pAb), obtained by is diluted 200 times with 15% ethanol (concentration of 8 mg/mL). The above mixture was energy-treated by 50-minutes exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 4 healthy donors by the electroencephalography.

Then, the resulting dilution, treated with the transformed biopotentials of the human brain, is diluted by successively reducing the initial concentration to obtain a C200 dilution in combination with an external mechanical effect—shaking each dilution.

Materials and methods of examination. The study included 20 patients with ADHD (15 boys, 5 girls) aged 6 to 12 years. 10 patients took the claimed medicament (group 1), 10—placebo (group 2). The diagnosis of ADHD was based on the criteria of the 10th revision of the International Classification of Diseases for Hyperdynamic (Hyperkinetic) Attention Deficit Disorder. The average age of patients included in the study in the 1st group was 9.1±0.36 years, and the 2nd placebo group was 8.9±0.32 years.

For the objectification of the patient's condition, the ADHD symptoms assessment scale (Conners scale) was used to fill in the researcher with the words of parents.

All patients had clinically pronounced manifestations of ADHD, which was confirmed by high initial scores on the scale: 30.8±1.77 in the 1st group and 33.6±1.33 in the 2nd group.

The drug was administered 10 drops 3 times a day, out of touch with food intake. The duration of treatment was 12 weeks. The dosage regimen, the route of administration and the duration of the course of treatment were the same for the stated drug and placebo. The examination of patients was carried out immediately before the start of treatment and in dynamics after 2, 4, 6, 8, 12 weeks of therapy.

Conclusions. The severity of the therapeutic effect after 12 weeks of therapy was more significant in the group of the claimed drug. Significant improvement with a decrease in the total score by 50% or more from the baseline on the symptom assessment scale of ADHD was observed in 3 (30%) patients, moderate improvement in 2 (20%). At the same time, in the majority of 4 (40%) patients from the placebo group, the improvement was moderate and only in 1 (10%)—significant. The average reduction of symptoms in the overall score of this scale was more pronounced 4 weeks after the start of treatment, and further significant differences with the placebo group remained and gradually became even more significant during the 2nd and 3rd months of therapy. Thus, the positive dynamics in the condition of the patients who received the therapy with the claimed Preparation was achieved compared to the placebo group after 1 month and continued to increase during the next two months of treatment. In the group of patients who received drug treatment, after 12 weeks of therapy, there was a significant decrease in the severity of manifestations as attention disorders (from 16.4±0.93 to 11.2±0.97, p<0.001, or by 31.7%) and hyperactivity/impulsiveness (from 14.4±1.17 to 8.4±0.93, p<0.001, or 41.7%). Thus, there was a tendency towards a more significant positive effect of the claimed medicament (Preparation) on the symptoms of hyperactivity/impulsivity.

After 1 month from the start of treatment in the Drug group, the total score on the Conners scale decreased by 23.4%—from 44.1±2.08 to 33.8±2.11 (p<0.001), whereas in the placebo group only by 6.2% from 49.7±2.33 to 46.6±2.50 (p>0.05). At the end of 3 months of treatment in the first group, there was a decrease in the scoring to 30.5±2.58 or by 30.8% (p<0.001) compared with the initial value. In the placebo group, this estimate reached over the same period 40.5±3.40 or decreased compared to the initial value by 18.5%.

Conclusions. The claimed medicament has a significant positive effect in the treatment of ADHD (with a combined type and moderate severity) in children: the appointment of the drug in a dosage of 10 drops 3 times a day for 12 weeks leads to a significant decrease in the severity of symptoms of ADHD compared with the placebo group.

### EXAMPLE 29

The study determined the efficacy and safety of the claimed medicament in tablet form (Preparation) in the treatment of attention deficit hyperactivity disorder (ADHD) in children.

Preparation 1 was administered as tablets containing a 1:3 v/v energy-treated mixture of the following:

A) the released-active form of polyclonal antibodies to S100 protein (S100 pAb), obtained by is diluted 150 times in an aqueous solvent (concentration of 3.5 mg/mL) (C150).

B) the released-active form of polyclonal antibodies to cannabinoid receptor type 1 (CB1 pAb), obtained by is diluted 5 times in an aqueous solvent (concentration of 5 mg/mL) (C5). The above mixture was energy-treated by 30-minutes exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 4 healthy donors by the electroencephalography.

Materials and methods of examination. The study included 57 children aged 6 to 9 years (40 boys and 17 girls) who attended day care centers and elementary schools of secondary schools.

Entry criterion:

1. The presence of clinical signs of hyperactivity that meet the criteria of ICD-10 (F 90);

2. Age—6-9 years;

3. Intellectual indicators are not lower than the average on the Wechsler scale (more than 90 points).

Children with behavioral disorders due to other mental disorders were excluded from the study. For 2 months, the main group of children (n=30) with clinical signs of GH received monotherapy for the claimed medicament in 2 tablet 3 times a day. Children from the control group, similar to the main group by clinical signs, sex and age (n=27), were administered Piracetam in standard age doses for the same period.

The assessment of the severity of GH according to the criteria of attention disorder, hyperactivity and impulsivity in the examined children was carried out using the SNAP-IV scale (Swanson J., 1992). The psychophysiological test TOVA (The Test of Variables of Attention), allowed to assess the degree of attention disorders (omissions of significant stimuli) and impulsivity (false presses of a button), information processing speed (reaction time) in relation to standard indicators (Greenberg L M, Waldman I D, 1993). The child's fine motor skills were evaluated using tests from the battery of A. R. Luria (1973), selected by testing on these age groups with the development of criteria for scoring points Zh.M. Glozman, et al. (2006).

The results of the study. At the first stage of the study, using the SNAP-IV scale (Swanson J., 1992) in children of the main and control groups, an initial assessment of the severity of GH was carried out separately for each of the criteria (attention disorder, hyperactivity and impulsivity). Repeated similar assessment was carried out after a two-month course of therapy with the declared Drug in the main group and Piracetam therapy in the control group. The obtained indicators were compared with statistical processing (Table 28).

TABLE 28
Dynamics of SNAP-IV in the main and control groups
after treatment with MMH-747 and piracetam

| | Children | | | |
|---|---|---|---|---|
| | with | active | | |
| | hyperactivity | treatment | observational | statutory |
| | before | group | group | indicator |
| | treatment | (Preparation) | (Piracetam) | (Swanson J., |
| Factor | (n = 57) | (n = 30) | (n = 27) | 1992) |
| disorder of | 2.76 ± 0.31 | 1.96 ± 0.11** | 2.52 ± 0.13 | 0.40-1.82 |
| attention | | | | |
| hyperactivity | 2.64 ± 0.29 | 1.44 ± 0.20** | 2.46 ± 0.11 | 0.27-1.59 |
| impulsivity | 2.40 ± 0.28 | 2.02 ± 0.27* | 2.36 ± 0.20 | 0.26-1.52 |

*p < 0.05,
**p < 0.01 - significance of differences when comparing indicators before and after therapy

As follows from Table 28, in the main group, the therapy with the claimed medicament resulted in a significant increase in the quality of attention, reduction of hyperactivity, and, to a lesser extent, impulsivity in the majority of patients in 21 children (70%, n=30). It should be noted the high reliability of differences in indicators in the main group before and after drug therapy, which is evidence in favor of its effectiveness. In the control group, therapy with Piracetam led to a positive dynamics of indicators in the absence of significant differences in the compared indicators.

The computer-aided testing of TOVA (“Test of Variables of Attention”) was represented by three groups of tests, which allow judging the degree of attention disorder (missing significant stimuli), impulsivity (false pressing a button), information processing speed (reaction time). Baseline indicators of children with GH before treatment were compared with those of children of the main group (treatment with the claimed Drug) and indicators of children of the control group (Piracetam therapy) after a two-month course of treatment. The results are presented in Table 29.

TABLE 29
Comparative dynamics of TOVA-test indicators in the main and control
group after the course of treatment with MMH-747 and piracetam.

| | | Children with | active | |
|---|---|---|---|---|
| | | hyperactivity | treatment | observational |
| | | before | group | group |
| | | treatment | (Preparation) | (Piracetam) |
| Psychophysiological data | | (n = 57) | (n = 30) | (n = 27) |
| Omissions of | First half of the | 19.1 ± 2.4 | 8.2 ± 1.2 ** | 13.5 ± 1.4 * |
| significant | test | | | |
| incentives, % | The second half | 29.5 ± 3.3 | 10.1 ± 1.3 ** | 16.9 ± 2.9* |
| | of the test | | | |
| false alarm, % | First half of the | 15.3 ± 2.2 | 7.7 ± 1.8 * | 12.6 ± 3.1 |
| | test | | | |
| | The second half | 29.2 ± 3.3 | 18.4 ± 1.9 * | 20.5 ± 2.9 |
| | of the test | | | |
| response | First half of the | 795 ± 103 | 722 ± 89 | 745 ± 87 |
| time, ms | test | | | |
| | The second half | 741 ± 126 | 625 ± 79 | 665 ± 77 |
| | of the test | | | |

* p < 0.05,
** p < 0.01 - significance of differences when comparing indicators before and after therapy

As follows from the table 29, a significant improvement in psychophysiological indicators reflecting the quality of attention (the test “Missing significant stimuli”) was achieved in the main and control groups. At the same time, a reliable reduction of impulsiveness (“False alarm” test) was noted only in the main group (Drug). The effect of the claimed medicament and Piracetam on the decision-making time (reaction time) was not statistically significant. Thus, the results of a psychophysiological study confirm the clinical data on the positive effect of the claimed medicinal preparation on the quality of attention, mental performance. Impulsiveness is also reduced.

The neuropsychological assessment of micromotorics in the main and control groups after the course of treatment with Drug and Piracetam also allowed us to establish a statistically significant improvement in both groups. The results are presented in table 30.

TABLE 30
Dynamics of indicators of the quality of fine motility
after treatment in the compared groups.

| | Children with | active | |
|---|---|---|---|
| | hyperactivity | treatment | observational |
| | before | group | group |
| | treatment | (Preparation) | (Piracetam) |
| Factor | (n = 57) | (n = 30) | (n = 27) |
| Total mark of | 2.24 ± 0.21 | 1.56 ± 0.21 ** | 1.62 ± 0.16 * |
| fine motor | | | |
| quality | | | |

* p < 0.05,
** p < 0.01 - significance of differences when comparing indicators before and after therapy.

Of particular note is the absence of side effects and any complications of therapy with the declared Drug during the course of this study; Piracetam in the comparison group in 3 children (11.1%) caused an increase in disinhibition, excitability, and in 7 (26%)—sleep disturbance (difficulty falling asleep, frequent awakenings).

Conclusions. In the course of this comparative study of the effectiveness of the claimed medicament in children with hyperkinetic disorder, an improvement in the child's behavior was achieved within 2 months with a reliable optimization of the indicators of attention quality and speed of information processing. At the same time, there was a reduction in hyperactivity, and, to a lesser extent, impulsiveness. The claimed medicament is statistically significantly superior to Piracetam in terms of its impact on the clinical symptoms of ADHD. In both groups, micromotor improvement was noted, but its more pronounced positive changes occurred in children of the main group. Reception within 2 months of the Drug did not cause side effects in the form of disinhibition, hyperkinesia, sleep disorders, which were observed when taking Piracetam.

### EXAMPLE 30

The study determined the efficacy and safety of the claimed medicament in tablet form (Preparation) in the treatment of cognitive impairment (voluntary attention, mental functions) in children with mental retardation in an open comparative study.

Preparation 1 was administered as tablets containing a 2:3 v/v energy-treated mixture of the following:

A) the released-active form of polyclonal antibodies to S100 protein (S100 pAb), obtained by mixing three different serial dilutions of an S100 pAb stock (matrix solution) diluted in an aqueous solvent (concentration of 2.5 mg/mL) The following dilutions were mixed at a 1:1:1 (v/v) ratio:

1) the stock (matrix) solution diluted 100¹² by (or to C12).

2) the stock (matrix) solution diluted by 100³⁰ times (or to C30), each time in combination with external mechanical treatment (shaking).

3) the stock (matrix) solution diluted by 100²⁰⁰ times (or to C200), each time in combination with external mechanical treatment (shaking).

B) the released-active form of polyclonal antibodies to cannabinoid receptor type 1 (CB1 pAb), obtained by mixing two different serial dilutions of a CB1 pAb stock (matrix solution) diluted in an aqueous solvent (concentration of 4 mg/mL) The following dilutions were used in the 1:1 (v/v) mixture:

1) the stock (matrix) solution diluted by 100¹² times (or to C12).

2) the stock (matrix) solution diluted by 100⁵⁰ times (or to C50), each time in combination with external mechanical treatment (shaking).

The above mixture was energy-treated by 2-hours exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 5 healthy donors by the electroencephalography. Then, the resulting dilution, treated with the transformed biopotentials of the human brain, is diluted by successively reducing the initial concentration to obtain a C100 dilution in combination with an external mechanical effect—shaking each dilution.

Material and methods of examination. Under the supervision there were 30 children (17 boys and 13 girls) 5-8 years old with mental retardation (borderline conditions) with a diagnosis of organic emotional-labile asthenic disorders with mild cognitive impairment; partial forms of delayed development of higher mental functions (VPF) with mild cognitive impairment; affective disorders with emotional lability in the syndrome of hyperactivity and attention deficit; astheno-neurotic disorders with a tendency to increase their severity—adaptation reactions with a vegetative and psychosomatic level of response. All patients prior to the study (for at least a week) and during it were excluded drugs with psychotropic activity.

The claimed medicament was administered 1 tablet 3 times a day for 8 weeks.

The control group consisted of 20 children (13 boys and 7 girls) with a similar clinical picture who did not receive specific medical treatment.

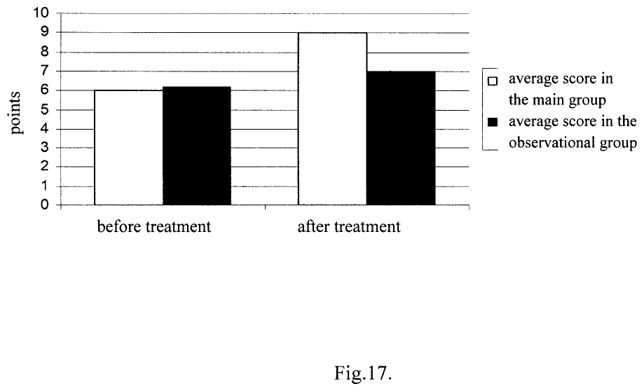

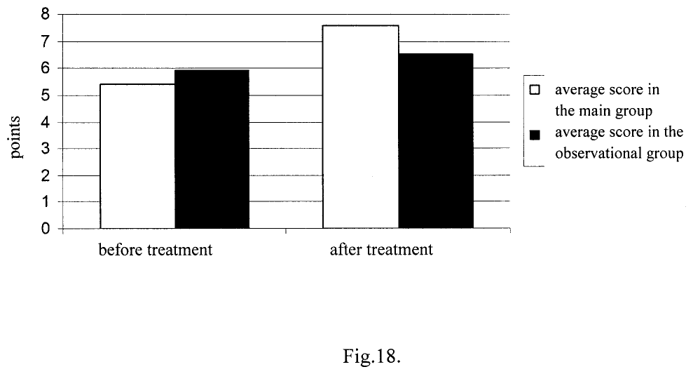

The results of the study. The results of the psychological study of cognitive functions were investigated by the following parameters of stability (FIG. 17) and distribution (FIG. 18) of attention were assessed by changing the average score in the “Encoding” subtest of the D. Wechsler battery, which in children of the main and control group was 6 before treatment (characterizing the rate is 10).

As can be seen on FIGS. 17 and 18 in children of the main group (Preparation), the average sustainability score of voluntary attention after 8 weeks increased from 6 to 9 points, indicating an improvement in the function of directional attention. In the control group, there was a less pronounced positive trend: the sustainability of voluntary attention increased from 6.3 to 7 points. The average score of attention distribution revealed in this method in the main group increased from 6 before the start of therapy to 7.5 points after its completion. This indicator in the comparison group was significantly lower—6 at the beginning and 6.5 points at the end of the study.

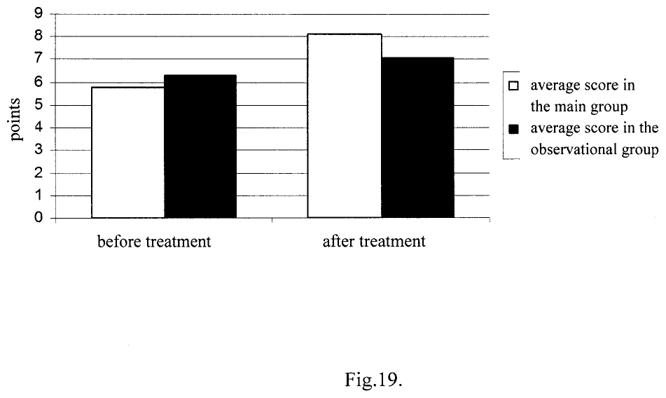

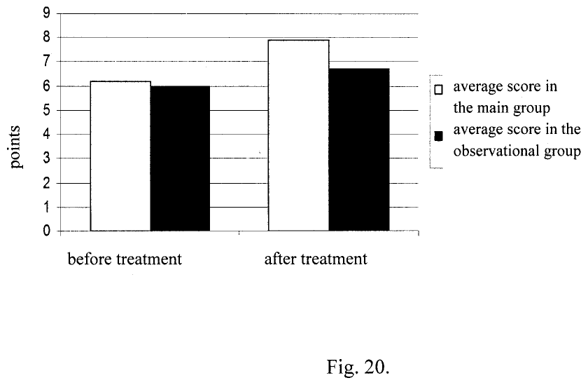

Since neuropsychological examination of many children with mental retardation reveals mnestic disorders, it is of particular importance to evaluate the effect of drug therapy on memory indications. In the study of the state of mnestic functions (auditory-speech and visual memory was assessed using the neuropsychological methodology “Luria-90” by E.G. Simernitskaya) in children in the main group taking the stated Preparation, a more noticeable positive development of the hearing-aural speech (FIG. 19) and visual memory was noted (FIG. 20), as well as a number of individual characteristics of visual memory: its volume, reproduction of the order of visual stimuli, interhemispheric transfer of visual information. The average score characterizing the state of hearing speech memory in children of the main group increased from 5.8 to 8.1 points, and the average score characterizing the state of visual memory from 6.2 to 7.9 points. In the control group, there was a slight tendency to improve the state of mnestic functions, however, these changes are less pronounced compared to the main group: hearing and speech memory improved in terms of the average score from 6.3 to 7.1, and visual memory from 6 to 6.7.

Conclusions. In children with mental retardation, the claimed medicament contributes to the improvement of cognitive functions—sustainability and attention distribution; improve mnestic functions—oral speech and visual memory. After the course of treatment with the drug, after 8 weeks, objectively using psychological methods and objectively, according to parents, there was a noticeable improvement in children's attention and memory, which had a positive effect on the success of children's education and their psychological adaptation in the children's team. It should be noted that no undesirable side effects of the drug were detected in any child; its good tolerance was noted throughout the duration of therapy.

### EXAMPLE 31

The study determined the efficacy and safety of the claimed drug in tablet form (Preparation) in the treatment of mild cognitive impairment in patients in the early recovery period of ischemic stroke.

Preparation 1 was administered as tablets containing a 3:1 v/v energy-treated mixture of the following:

A) the released-active form of polyclonal antibodies to S100 protein (S100 pAb), obtained by is diluted 60 times in an aqueous solvent (concentration of 8 mg/mL)

B) the released-active form of polyclonal antibodies to cannabinoid receptor type 1 (CB1 pAb), obtained by is diluted 200 times in an aqueous solvent (concentration of 5 mg/mL). The above mixture was energy-treated by 1-hours exposure to an electric current resulting from a potential difference applied to electrodes immersed in the stock (matrix) solution and proportional in amplitude to the amplified total composite signal constituted by the sum of outputs—the differences in the bioelectric potentials of the human brain, recorded from 2 healthy donors by the electroencephalography.

Material and methods of examination. The study involved 4 people who underwent a single stroke at the ischemic type, from 3 to 6 months old, with cognitive impairments (less than 26 points on the MoCA scale) and moderate activity in everyday life (61-80 points on the Bartel scale). The average age of patients was 60.75±7.37 years, all patients were male.

Patients were prescribed the claimed drug Preparation 2 tablets 2 times a day, daily for 24 weeks while receiving basic therapy (antihypertensive drugs, diuretics, antiplatelet agents, anticoagulants, statins, drugs for the treatment of diabetes).

The dynamics of cognitive impairment was assessed on the MoCA scale after 24 weeks of receiving the studied therapy compared to the period before the start of the drug.

The results of the study. The results of treatment are presented in table 31.

TABLE 31
Dynamics of indicators on a scale of MoCA before
and after treatment with the drug MMN-MAP.

| | MoCA | MoCA | |
|---|---|---|---|
| | before | after | |
| No | treatment | treatment, | |
| patient | points | points | Subjective |
| 001 | 19 | 25 | Improving memory, improving mental |
| | | | efficiency |
| 002 | 20 | 27 | Increased concentration, improved |
| | | | audio-verbal memory |
| 003 | 23 | 29 | Improving well-being, reducing the |
| | | | severity of cognitive impairment |
| 004 | 14 | 22 | Significant improvement in cognitive |
| | | | functions, better tolerance to |
| | | | stress, reduced need for care |

When assessing the severity of cognitive impairment in all patients (100%), a significant increase in the number of points on the MoCA scale was observed, which was expressed in improving memory, attention, and increasing mental and physical performance. In one patient, recovery of cognitive functions from the degree of pronounced cognitive decline to the degree of moderate cognitive impairment, manifested in a reduced need for care, was noted.

Findings. In this study, the claimed medicinal drug showed its positive effect on cognitive functions (improvement of memory and attention, learning and memory), which proved to be an increase in mental performance after 28 days of taking the drug.

1. A medicament for treating disorders of functions of organs or tissues, wherein the medicament comprises an original drug substance or a release-active form of the drug substance;
wherein said drug substance or the release-active form of the drug substance is treated by passing an electric current;
wherein the electric current is applied to at least two electrodes immersed in the drug substance or the release-active form of the drug substance;
wherein the electric current is proportional to bioelectric potentials measurements obtained from a normally functional organs or tissues.

2. (canceled)

3. The medicament of claim 1, wherein the treated drug substance or the release-active form of the drug substance is subjected to successive multiple dilutions in a solvent selected from the group comprising water and a mixture of water and ethanol, complemented by external treatment.

4. The medicament of claim 1, wherein the bioelectric potentials are measured by using an electrophysiological method on normally functional organs or tissues.

5. The medicament of claim 1, wherein the bioelectric potentials are measured taken reacting from the organs or tissues of one or more healthy donors.

6. The medicament of claim 5, wherein the bioelectric potentials measured from more than one healthy donor are summed before use for treating the drug substance or the release-active form of the drug substance.

7. The medicament of claim 1, wherein the potential difference applied to the electrodes immersed in the drug substance or the release-active form of drug substance is proportional in amplitude to the amplified resultant signal produced by the summing of the bioelectric potential measurements obtained by the electrophysiological method on the normal functioning organs or tissues.

8. The medicament of claim 1, wherein the organ is that in a living subject.

9. The medicament of claim 1, wherein the organ is an isolated one.

10. The medicament of claim 1, wherein the organ is one selected from a group containing heart, gastrointestinal tract, muscle, retina, skin, brain or circulatory system.

11. The medicament of claim 1, wherein the bioelectric potentials are measured by using one of the procedures selected from a group containing electrocardiography, electrogastroenterography, electromyography, electroretinography, electroencephalography, EDA measurement or impedance plethysmography.

12. The medicament of claim 10, wherein the disordered function is disorder of a heart function, gastrointestinal function, muscle function, brain function, skin function or circulatory function.

13-30. (canceled)

31. The medicament of claim 1, wherein the drug is available in a solid dosage form comprising a technologically appropriate efficient amount of a neutral carrier saturated with liquid form of the treated drug substance or liquid release-released-active form of the drug substance, and pharmaceutically acceptable excipients.

32. The medicament of claim 31, wherein the treated drug substance or release-active form of the drug substance is subjected to further successive multiple dilutions in a solvent selected from the group comprising water and a mixture of water and ethanol before being introduced onto the neutral carrier solid for subsequent saturation.

33. The medicament of claim 32, wherein the successive multiple dilutions are accompanied by external mechanical treatment.

34. The medicament of claim 1, wherein the drug substance is an antibody.

35. The medicament of claim 34, wherein the antibody is a monoclonal, polyclonal, or natural antibody.

36. The medicament of claim 1, wherein the drug substance is chosen to be antibodies to interferon gamma, S-100 brain protein, angiotensin II receptor, endothelial NO-synthase, human cannabinoid receptor—used either individually or as a combination.

37. The medicament of claim 1, wherein the drug substance is chosen to be drugs or drug combinations.

38.-66. (canceled)

67. A method of preparing the medicament of claim 1, wherein the drug substance or the release-active form of the drug substance is treated by passing an electric current;
wherein the electric current is applied to at least two electrodes immersed in the drug substance or the release-active form of the drug substance;
wherein the electric current is proportional to bioelectric potential measurements obtained from normally functional organs or tissues.

68. The method of claim 67, wherein the treated drug or a treated release-active form of the drug substance is further subjected to successive multiple dilutions in a solvent selected from the group comprising water and a mixture of water and ethanol.

69. The method of claim 68, wherein the successive multiple dilutions are accompanied by external mechanical treatment, wherein external mechanical treatment involves shaking the diluted medicament after every dilution.

70. The method of claim 69, wherein the bioelectric potentials are measured by using an electrophysiological method for recording bioelectric potentials from a normally functional organs or tissues.

71. The method of claim 70, wherein the electrophysiological method for recording bioelectric potentials is one of the procedures in a group containing electrocardiography, electrogastroenterography, electromyography, electroretinography, electroencephalography, EDA measurement or impedance plethysmography.

72. The method of claim 70, wherein the obtained potentials are amplified in each channel and the amplified outputs are converted into digital form and transmitted as digitized signal files to the memory of a programmable digital signal summing unit—signal summer, where the digital signals are added together into one composite digital signal bearing information on the sum of potential differences from all recorded electrodes, which is afterwards input to the digital-to-analog converter of an arbitrary waveform generator, where it is converted back into a summed analog signal to generate output signals in the form of voltage (potential difference) that approximate the waveform of the summed bioelectric potential difference, which are then fed onto at least two current electrodes immersed in the container containing the drug substance or the release-active form of the drug substance.

73. The method of claim 70, wherein the organ is that in a living subject.

74. The method of claim 70, wherein the organ is an isolated one.

75. The method of claim 70, wherein the organ is one selected from a group containing heart, gastrointestinal tract, muscle, retina, skin, brain or circulatory system.

76. The method of claim 67, wherein the drug substance is an antibody, wherein the antibody is a monoclonal, polyclonal or a natural antibody.

77. The method of claim 67, wherein the drug substance is chosen to be antibodies to interferon gamma, S-100 brain protein, angiotensin II receptor, endothelial NO-synthase, human cannabinoid receptor—used either individually or as a combination.

78. The method of claim 67, wherein the drug substance is chosen to be a drug or drug combinations.

79. The method of claim 67, wherein the release-active form of the drug substance is obtained by successive multiple dilutions in a solvent selected from the group comprising water and a mixture of water and ethanol, complemented by external treatment.

80. The method of claim 79, wherein the external treatment is shaking of every dilution.

81. A method of using the medicament of claim 1 for treatment of diseases accompanied by disorders of functions of a body organ or tissue, and for known indications for the drug substance or the release-active form of the drug substance.

82. The medicament of claim 1, wherein the release-active form of the drug substance is obtained by successive multiple dilutions in a solvent selected from the group comprising water and a mixture of water and ethanol, complemented by the external treatment.
